(19) United States
(12) Patent Application Publication
(10) Pub. No.: US 2025/0154467 A1
(43) Pub. Date: May 15, 2025

(54) CREATION OF VASCULARIZED BIOLOGICAL STRUCTURES

(71) Applicant: The Board of Trustees of the Leland Stanford Junior University, Stanford, CA (US)

(72) Inventors: Oscar J. Abilez, Redwood City, CA (US); Huaxiao Yang, Redwood City, CA (US); Joseph C. Wu, Stanford, CA (US)

(21) Appl. No.: 18/838,211

(22) PCT Filed: Feb. 27, 2023
(86) PCT No.: PCT/US2023/013997

§ 371 Date: Aug. 13, 2024

Related U.S. Application Data
(60) Provisional application No. 63/314,958, filed on Feb. 28, 2022.

(51) Int. Cl. C12N 5/077; G01N 33/50

(52) U.S. Cl. CPC C12N 5/0652; G01N 33/5082; C12N 2500/38; C12N 2501/10; C12N 2501/11; C12N 2501/115; C12N 2501/119; C12N 2501/12; C12N 2501/13; C12N 2501/135; C12N 2501/15; C12N 2501/155; C12N 2501/165; C12N 2501/999; C12N 2506/45; C12N 2513/00; C12N 2535/10

(57) ABSTRACT

Compositions and methods are provided relating to in vitro differentiation of vascularized two-and three-dimensional biological structures, which may be referred to herein as vascularized organoids (VOs). Vascularization in the organoids is spatially organized, and can include hierarchical branching of large vessels to small vessels. Features of the vascularized organoids include vascular tissues having the same branching structure and self-organization as seen in vivo; and physiologically relevant spatial organization within endocardial, myocardial, epicardial, and/or progenitor cells. The organoids also have inherent vascular beds that can be anastomosed immediately with host vasculature beds, thereby improving transplantation efficiency by rapid oxygenation from blood flowing through their pre-incorporated vascular beds.

### CROSS REFERENCE TO OTHER APPLICATIONS

This application claims the benefit of U.S. Provisional Application No. 63/314,958, filed Feb. 28, 2022, the contents of which are hereby incorporated by reference in its entirety.

### STATEMENT REGARDING FEDERALLY SPONSORED RESEARCH OR DEVELOPMENT

This invention was made with Government support under contracts HL130608, HL150693, HL141371, HL146690, HL145676, and HL141084 awarded by the National Institutes of Health; and contract 18POST34030106 awarded by the American Heart Association. The Government has certain rights in the invention.

### BACKGROUND

Human pluripotent stem cells (hPSCs), including human embryonic stem cells (hESCs) and human induced pluripotent stem cells (hiPSCs) can differentiate into any cell type in the body. Through specification of mesoderm and endoderm, and then progenitor intermediates, hPSCs can be differentiated into cardiomyocytes (CMs), hepatocytes (HCs) and individual cardiovascular cell types, including endothelial cells (ECs), smooth muscle cells (SMCs), fibroblast cells (FBs), endocardial cells (ENDOs), and epicardial cells (EPIs). However, the lack of a prospective and directed in vitro strategy to create de novo vasculature with robust branching and hierarchical organization within organoids, is a major bottleneck in the stem cell field. Very little is known about this process in humans, due to ethical restrictions and the technical difficulty of obtaining embryos at such early stages of development.

Currently lack of robust vascularization of mammalian (both animal and human) biological structures, including stem cell-derived cells, tissues, organs, and organoids is a major bottleneck for having representative in vitro models for developmental biology research, disease modeling, drug efficacy and toxicity testing, and regenerative medicine. Prior art engineered tissues have been created that have vascular cell types, e.g. ECs, SMCs, and FBs, but which lack the branching structure seen in in vivo vascular beds.

The disclosure herein addresses methods for generating vascularized biological structures in vitro.

### SUMMARY

Compositions and methods are provided relating to in vitro differentiation of vascularized two-and three-dimensional biological structures, which may be referred to herein as vascularized organoids (VOs). Vascularization in the organoids is spatially organized, and can include hierarchical branching of large vessels to small vessels. Features of the vascularized organoids include vascular tissues having the same branching structure and self-organization as seen in vivo; and physiologically relevant spatial organization within endocardial, myocardial, epicardial, and/or progenitor cells. The organoids also have inherent vascular beds that can be anastomosed immediately with host vasculature beds, thereby improving transplantation efficiency by rapid oxygenation from blood flowing through their pre-incorporated vascular beds.

The combinations of vascular-inducing factors exemplified herein for the in vitro generation of cardiac VOS (CVOs) and hepatic VOS (HVOs) are also useful for creating vasculature in other organ systems, e.g., in combination with cell-specific differentiation protocols to achieve vascularization in other organ systems. The vascularization process can be applied to a 3D system and integration with the published 3D systems can provide for recapitulation of cardiogenesis with high fidelity. Organoid vascularization provides improvements to such organ systems by reducing necrosis in the center of organoids where oxygen tension is low, provides for larger organoid growth in vitro for improved systems for disease modeling, drug toxicity testing, and drug discovery, and can increase the viability of implanted organoids for regenerative medicine applications in vivo.

In some embodiments of the methods to generate vascularized organoids, geometrically micropatterned pluripotent stem cells (PSCs), e.g. human PSCs, such as human embryonic stem cells (hESCs) or human induced pluripotent stem cells (hiPSCs), are used to initiate a culture. The PSCs are subsequently differentiated into germ layers, progenitor cell types, and differentiated cell types using biochemical growth factors and small molecules. Biophysical/mechanical, electrical, optical, and other biochemical factors may be used to augment the vascularization process. Spatial micropatterning of PSCs enables repeatable and scalable formation of spatially organized germ layers and differentiated cells. The VOs may be initiated with various pluripotent stem cells, wherein certain embodiments the PSC are genetically modified to comprise one or more reporter genes, e.g., one, two, three, four or more fluorescent reporters, which reporters enable temporal identification of germ layer, progenitor, and/or cardiovascular cell types in situ without disturbing the architecture of developing VOs.

In one embodiment, cultures are initiated with hPSCs micropatterned on the surface of a culture vessel. Micropatterning in culture may comprise introducing a stencil or equivalent to a culture well, wherein the stencil comprises holes of from about 1 mm, about 2 mm, about 3 mm, up to about 10 mm diameter or an equivalent size in a non-circular pattern, up to about 10 mm. A suitable matrix is poured over the stencil, and the PSC are allowed to attach to the matrix. After removal of the stencil, micropatterned cells are attached on the islands created by the holes of the stencils. In alternative embodiments, other suitable means can be used to pattern a matrix on the surface of a culture vessel, i.e., dispensing a matrix solution from a printer, and the like, preprinting surfaces, etc. where the hPSC are then allowed to adhere. The matrix may be provided in any convenient geometry, e.g. a circle, oval, square, rectangle, etc. When the hPSC become confluent, e.g., after from about 1 to 5 days, additional matrix medium can be added to allow the colonies to migrate beyond the initial boundaries.

The micropatterned colonies are co-differentiated into cells of interest using biochemical growth factors and small molecules suitable for the desired cell types, utilizing a series of steps specific media for in vitro differentiation. The differentiation protocol allows for formation of the three primary germ layers, comprising the endoderm, mesoderm, and ectoderm, and subsequent differentiation to create vascularized organoids. By controlling the ratio of each of the germ layers through simultaneous induction of the desired germ layers, vascularized structures that derive from the germ layers are generated. For example, cardiac vascularized organoids (cVOs) are mesoderm-derived; hepatic vascularized organoids (hVOs) are mesoderm-and endoderm-derived; and neural vascularized organoids (hVOs) are mesoderm-and ectoderm-derived organoids.

In methods of generating cardiac VOs and cell compositions thus generated, the vascularized organoids show simultaneous in vitro co-differentiation of atrial and ventricular cardiomyocytes (CMs), arterial and venous endothelial cells (ECs), various smooth muscle cell (SMC) subtypes, various fibroblast (FB) subtypes, endocardial cells (ENDOs), epicardial cells (EPIs), along with other cell types and various extracellular matrix (ECM) proteins, achieved by micropatterning hPSCs and exposing them to a cocktail of cardiac-and vascular-inducing factors. The cVOs are both vascularized and organized. Specific combinations of factors are further discussed herein.

In methods of generating hepatic VOs and cell compositions thus generated, the vascularized organoids show simultaneous in vitro co-differentiation of hepatocytes, and a branching network of endothelial cells (ECs) and various smooth muscle cell (SMC) subtypes, achieved by micropatterning hPSCs and exposing them to a cocktail of hepatic-and vascular-inducing factors. The cVOs are both vascularized and organized. Specific combinations of factors are further discussed herein.

In some embodiments a kit is provided comprising suitable growth factor cocktails and micropatterned tissue culture plates for use in generating vascularized structures of the disclosure.

In some embodiments, 2D and 3D vascularized tissues/organoids are provided for use in performing drug efficacy and toxicity testing. In such embodiments of the invention, methods are provided for determining the activity of a candidate agent on an organoid, the method comprising contacting the candidate agent with one or a panel of in vitro generated vascularized organoids as described herein; and determining the effect on a phenotype of the vascularized organoids.

In some embodiments, 2D and 3D vascularized tissues/organoids are provided for use in disease modeling.

In some embodiments, 2D and 3D vascularized tissues/organoids are provided as a biologic therapy for replacing lost and/or damaged tissue/organs for regenerative medicine applications.

In some embodiments, in vitro cell cultures of vascularized organoids are provided.

### BRIEF DESCRIPTION OF THE DRAWINGS

FIGS. 1A-1K. Micropatterning of hPSCs results in spatially organized cardiomyocytes. (A) A single circular 2 mm hPSC micropattern in each well of a multi-well plate is created from a silicone stencil. Differentiation begins when hPSC micropatterns are 100% confluent, typically 2-3 days post seeding. (B) Phase imaging showing 2, 4, and 6 mm undifferentiated hPSC micropatterns. Scale bars, 2, 4, 6 mm, respectively. (C) Confocal imaging showing 2, 4, and 6 mm hPSC micropatterns are pluripotent by immunostaining for OCT4 (green), SOX2 (red), and nuclear staining with DAPI (blue). White arrows show areas of incomplete hPSC confluence in 6 mm micropatterns. Scale bar, 1 mm. (D) Photograph showing 2 mm hPSC micropatterns (blue) in the centers of four wells of a 48-well dish. Scale bar, 9 mm. (E) Phase imaging showing cardiomyocyte (CM) differentiation of a 6 mm hESC-TNNT2-GFP micropattern from Day 0-9 (DO-9). Scale bar, 6 mm. (F) Fluorescence imaging of (E) showing GFP+ CMs (green). Scale bar, 6 mm. (G) An array of circular 2 mm hPSC micropatterns in each well of a multi-well plate is created from a silicone stencil. For a 6-well dish, up to 77 hPSC micropatterns can be created per well for a total of 462 micropatterns. (H) Photograph showing an array of 77 hPSC micropatterns in 1 well of a 6-well dish (left); phase imaging showing undifferentiated hPSC micropatterns (middle); fluorescence imaging showing organized and arrayed GFP+ CMs. Scale bars, 10, 4, 4 mm, respectively. (I) Fluorescence imaging showing an array of four 2 mm hPSC micropatterns are pluripotent by immunostaining for OCT4 (green), SOX2 (red), and nuclear staining with DAPI (blue). The total fluorescence area of DAPI of each micropattern is shown in yellow. Scale bar, 2 mm. (J) The actual total fluorescence area of the micropatterns correlates with micropattern count (R²=0.989) (n=4 per micropattern count). Error bars +/− SD. (K) The actual total fluorescence area of the micropatterns correlates with the theoretical total micropattern area (multiples of pi*r\hat{ }2, where r is the radius of each micropattern) (R²=0.99) (n=4 per micropattern count). Error bars +/− SD.

FIGS. 2A-2K. Micropatterning gives rise to organized germ layer and cardiovascular progenitor formation. (A) The hESC-RUES-GLR (germ layer reporter) cell line (Martyn et al., 2018) expresses SOX2, BRA, and SOX17 to identify undifferentiated cells and, upon differentiation, ectoderm, mesoderm, and endoderm arranged in stereotypical concentric rings as shown. The hESC-NKX2-5-eGFP cell line (Elliott et al., 2011) identifies cardiovascular progenitors in the middle ring arising from mesoderm. The hESC-3R cell line identifies SMCs, CMs, and ECs arising from cardiovascular progenitors. (B) Confocal imaging at day 0 showing SOX2 expression throughout a single undifferentiated micropattern. Scale bar, 1 mm. (C) Confocal imaging showing unpatterned (left) and micropatterned (right) RUES-GLR hESCs at day 6 with CHIR 4, 6, and 8 uM added at day 0. Unpatterned hESCs give rise to disorganized germ layers (BRA+, SOX17+, SOX2+ expression) and SOX2 is expressed at all CHIR concentrations. In contrast, micropatterned hESCs give rise to organized germ layers with minimal SOX2 expression. For CHIR 4 uM, micropatterned hESCs give rise to a BRA+ mesodermal central area surrounded by a SOX17+ endodermal ring, with no SOX2 ectodermal expression. Scale bar, 1 mm. (D-E) Representative phase imaging showing differentiation of unpatterned (n=2) and micropatterned (n=2) RUES-GLR hESCs over 11 days after addition of CHIR 4 uM at day 0 and IWR 5 uM at day 3. Scale bar, 2 mm. The unpatterned group has higher starting numbers of cells and the numbers increase over time for both groups. Shaded bands +/−1 SD. (F-G) Representative fluorescence imaging shows temporal expression of the mesodermal marker BRA of RUES-GLR hESCs from (D). Scale bar, 2 mm. Unpatterned BRA peaks around day 8, while micropatterned BRA peaks around day 11. Shaded bands +/−1 SD. (H-I) Representative fluorescence imaging shows temporal expression of the mesodermal marker BRA and the endodermal marker SOX17 of RUES-GLR hESCs from (D). Scale bar, 2 mm. Unpatterned SOX17 peaks around day 4, while micropatterned SOX17 peaks around day 11, coinciding with BRA peak expression. Shaded bands +/−1 SD. (J) The hESC-NKX2-5-eGFP cell line expresses eGFP under the cardiovascular progenitor transcription factor NKX2-5 as previously described (Elliott et al., 2011). (K) A single NKX2-5-eGFP micropattern at day 0 (left) differentiated over 10 days showing organized ring formation of eGFP+ cardiovascular progenitors leading to beating cardiomyocytes (right). Scale bar, 2 mm.

FIG. 3A-3S. Creation of a triple-reporter line enables screening differentiation conditions leading to cVO formation. (A) The hESC-3R line comprises the TNNT2 promoter driving GFP to identify CMs, the CDH5 (VE-Cadherin) promoter driving mOrange to identify ECs, and the TAGLN (SM22a) promoter driving CFP to identify SMCs. (B) Confocal fluorescence imaging and immunostaining confirms the differentiation of hESC-3R into CMs (co-expression of Troponin-T with TNNT2-GFP) (top row), ECs (co-expression of VE-Cadherin with CDH5-mOrange) (middle row), and SMCs (expression of TAGLN-CFP) (bottom row). Nuclei are stained with DAPI. Scale bar, 50 um. (C) Schematic showing timeline, stage, cell types, geometry, media, and growth factors for co-differentiating CMs, ECs, SMCs, leading to formation of cVOs. (D) A total of thirty-four (34) differentiation conditions (n=175 total micropatterns, n=4-6 per condition) were screened for those giving the highest simultaneous co-differentiation of CMs, ECs, and SMCs. Half of the conditions used CHIR 4 uM (n=88) and half used 5 uM (n=87). Overall, 5 uM gave the most CMs, ECs, and SMCs. Student's t-test, ***p<0.0001. (E-H) CM formation is highest in Condition 32* and is also shown for just conditions using CHIR 5 uM (even numbered Conditions 2-34). CM formation begins around day 8, and at day 12 is significantly higher for Condition 32 (n=6) compared to Control** (n=4). Student's t-test, ***p<0.0001. Error bars and shaded bands +/−1 SD. (I-L) EC formation is highest in Condition 32 and is also shown for just conditions using CHIR 5 uM (even numbered Conditions 2-34). SMC formation begins to increase around day 8, and at day 12 is significantly higher for Condition 32 (n=6) compared to Control** (n=4). Student's t-test, ***p<0.0001. Error bars and shaded bands +/−1 SD. (M-P) SMC formation is highest in Condition 32 and is also shown for just conditions using CHIR 5 uM (even numbered Conditions 2-34). EC formation is already apparent at day 3 and begins to increase around day 5, a few days before CM formation, and at day 12 is significantly higher for Condition 32 (n=6) compared to Control** (n=4). Student's t-test, ***p<0.0001. Error bars and shaded bands +/−1 SD. (Q) At day 12, VEGF alone gave the most ECs compared to Control (n=10, ***p<0.0001), to FGF2 alone (n=10, ***p<0.0001), and to SB alone (n=8, *p<0.05), while VEGF had no statistical (ns) effect on CM formation. One-way ANOVA with Tukey's test for multiple comparisons for GFP and mOr channels separately. (R-S) Representative time-lapse imaging shows CM, EC, and SMC formation over 12 days for Control (Condition 2) and cVO (Condition 32). In the cVO sample, note ring formation of CMs, with outward radiation of ECs, and central SMCs. *Condition 32 (cVO) consists of the following small molecules and growth factors: CHIR, 5 uM (day 0); FGF2, 5 ng/ml (days 0, 7, 9, 11, 13); IWR, 5 uM (day 3); VEGF, 50 ng/ml (days 5, 7, 9, 11, 13); SB 10 uM (days 7, 9, 11); ANG2, 50 ng/ml (days 5, 7); ANG1, 50 ng/ml (days 9, 11); PDGFBB, 10 ng/ml (days 7, 9, 11, 13); and TGFβ, 2 ng/ml (days 13). **Condition 2 (Control) is the standard cardiomyocyte differentiation with only the factors CHIR, FGF2, and IWR. Error bars +/−1 SD.

FIGS. 4A-4N. cVOs comprise spatially and temporally self-organized cardiovascular cell types. (A) Confocal phase (left) and fluorescence (middle) imaging shows the differentiation of a single hESC-3R micropattern over 16 days (˜3 weeks of in vivo human development) into a cVO containing CMs (green), ECs (orange), and SMCs (blue). Nuclei are labeled with DRAQ5 nuclear stain (red). Note concentric organization of cell types and EC branching. This plane corresponds to section A′-A′, near the well bottom (right). Scale bar, 2 mm. (B) Individual confocal channels showing CMs (green) (left), ECs (orange) (middle), and SMCs (blue) (right). Scale bar, 2 mm. (C) Confocal fluorescence images showing hESC-3R CMs co-express Troponin-T (red) with TNNT2-GFP (green). Nuclei are labeled with DAPI (blue). Scale bar, 50 um. (D) Confocal fluorescence images showing hESC-3R ECs co-express PECAM (red) with CDH5-mOr (orange). Nuclei are labeled with DAPI (blue). Scale bar, 50 um. (E) Confocal phase (far left) and fluorescence imaging shows an array of hESC-3R micropatterns differentiated over 16 days into cVOs containing concentrically self-organized CMs (green), ECs (orange), and SMCs (blue). Note individual micropatterns give rise to an array of independently-formed cVOs despite sharing the same media. Scale bar, 2 mm. (F) Enlarged area from red box in (E) showing concentric organization of CMs, ECs, and SMCs, with EC branching. This plane corresponds to section B′-B′ in (A). Scale bar, 2 mm. (G) 3D cVO differentiated over 16 days on a micropatterned substrate with a stiffness of 16 kPa showing CMs (green) surrounded by a branching network of ECs (orange). SMCs were not imaged. Scale bar, 1 mm. (H) Fluo-4 calcium CM labeling of a Control (top) and cVO (bottom). Calcium transient rates of Controls are higher than cVOs (right). Note samples were differentiated from hiPSCs to avoid spectral overlap of Fluo-4 with GFP from the hESC-3R line. Scale bar, 2 mm. (I) Beat rate of Controls (n=15) and cVOs (n=16) increases with isoproterenol (0, 1, and 10 uM) treatment, with Control rates consistently higher than cVO rates at each concentration. Two-way ANOVA with Tukey's test for multiple comparisons, *p<0.05 and *p<0.01. (J) hESC-3R EC nitric oxide (NO) secretion is higher in cVO ECs compared to Controls. Student's t-test, *p<0.05. (K) Temporal bulk RNA-sequencing (RNA-seq) was performed for undifferentiated hESC-3R micropatterns (blue, n=3), Control differentiation (black, n=3), and cVO differentiation (red, n=3). Principal component analysis (PCA) shows differences in differentiation trajectories between the three groups over 16 days (D0-16). Note trajectory divergence begins at day 5, when vascular induction begins. (L) Temporal RNA-seq weighted gene co-expression network analysis (WGCNA) heat map showing clusters of Pluripotency, Mesoderm, CV progenitor, Cardiomyocyte, Endothelial, Smooth Muscle, Fibroblast, and Vascularization Genes over 16 days (DO-16) for hESC-3R (blue, n=3), Control (black, n=3), and cVO (red, n=3) groups. (M) Comparison of select gene groups most notably show that EC and Notch-Delta-Jag gene expression (dashed rectangles) is higher for cVOs and diverges from Controls around day 5 (dashed arrows), when vascular-inducing factors are added to the cultures. Other gene groups trend as expected. (N) 3D cVOs, day 14 created with varying amounts of CHIR (6.0-8.5 uM) with 7.0 uM giving the most CMs (green) and ECs (orange). n=15-16 per group. Scale bar, 1 mm.

FIGS. 5A-5T. Single-cell RNA-sequencing reveals multiple vascular, endocardial, myocardial, and epicardial cell types in cVOs. (A) Control UMAP showing 8 clusters containing cardiomyocytes (CM1, CM2, CM3), endothelial cells (EC), smooth muscle cells (SMC1, SMC2,SMC3, SMC4), fibroblasts (FB1, FB2), epicardial cells (EPI1, EPI2), precursor cells (PRE), hepatic cells (HC), and neural cells (NC). (B) Control composition and lineage relationships of cellular subtypes from (A). (C) cVO UMAP showing 8 clusters containing cardiomyocytes (CM), endothelial cells (EC), smooth muscle cells (SMC1, SMC2, SMC3), fibroblasts (FB1, FB2, FB3), epicardial cells (EPI1, EPI2, EPI3), precursor cells (PRE), proliferating cells (PROLIF), hepatic cells (HC), and epithelial cells (EPT). (D) cVO composition and lineage relationships of cellular subtypes from (A). (E) cVO heatmap showing cell types in UMAP clusters in (C) according to scaled, log-normalized differentially expressed genes. (F) 6.5 PCW human heart t-SNE showing 14 cell types. cVOs share 9 of 14 cell types (dashed rectangles). PCW, post-conception week. (G-P) cVO violin plots showing expression of CMs, ECs, SMCs, FBs, EPIs, HCs, PROLIFs, ECM, NOTCH pathway, and VEGF pathway genes in corresponding UMAP clusters in (C). (Q) cVO UMAP showing CM atrial (MYH6+, MYL7+) and ventricular (MYH7+, MYL2+) subtypes. (R) cVO UMAP showing EC arterial (EFNB2+, UNC5B+, SOX17+, CXCR4+, DLL4+) and venous (NRP2+, APLNR+, TEK+, DAB2+, EPHB4+) subtypes. Note, the arterial subtype expresses a subset of venous genes and vice-versa. (S) cVO UMAP showing EC (CDH5) and endocardial (TOP2A+, UBE2C+, RRM2+, CDK1+, KIAA0101+) subtypes. (T) cVO UMAP showing SMC (TAGLN+, ACTA2+), FB (TCF21+), and EPI (TOP2A+, UBE2C+, RRM2+, BIRC5+, CDK1+, KIAA0101+) subtypes.

FIGS. 6A-6Q. Inhibition of NOTCH, BMP, and VEGF pathways disrupts vasculature within cVOs. (A) NOTCH-DLL-JAG receptor-ligand pair interactions between CMs, ECs, and SMCs are shown for Control, cVO, and an in vivo 6.5 PCW human heart (Asp et al., 2019). (Left) Control shows Notch receptors and Jag ligands, but no Delta ligands, along with their interactions (green and red shaded arrows). (Middle) cVO shows more receptors, ligands, and interactions. (Right) in vivo 6.5 PCW heart shows the most receptors, ligands, and interactions. Bolded genes show differences between the cVO and heart groups, with the heart expressing more Notch receptors. PCW, post-conception week. (B-D) DAPT, a NOTCH pathway antagonist, significantly decreased cVO EC formation over 16 days at 1 uM (n=6, **p<0.005) and 10 uM (n=6, **p<0.005) compared to 0 uM (Control) (n=12). There was no significant (ns) difference between 1 and 10 uM. One-way ANOVA with Tukey's test for multiple comparisons. Shaded bands and error bars +/−1 SD. Scale bar, 2 mm. Note, DAPT and Dorsomorphin were tested together and share the same Control group. (E-G) Dorsomorphin, a BMP pathway antagonist, significantly decreased cVO EC formation over 16 days at 0.1 uM (n=6, ***p<0.0001) and 1.0 uM (n=6, ***p<0.0001) compared to 0 uM (Control) (n=12). There was no significant (ns) difference between 0.1 and 1.0 uM. One-way ANOVA with Tukey's test for multiple comparisons. Shaded bands and error bars +/−1 SD. Scale bar, 2 mm. (H-J) Angiostatin, a known VEGF pathway antagonist, disrupted and significantly decreased cVO EC formation over 16 days at 0.1 ug/mL (n=5, ***p<0.0005) and 1.0 ug/mL (n=6, ***p<0.0005) compared to 0 ug/mL (Control) (n=10). There was no significant (ns) difference between 0.1 and 1.0 ug/mL. At day 16 and 0.1 ug/mL, ECs congregated in the center area of starting micropatterns (white dashed circle) while at 1.0 ug/mL, ECs congregated at the periphery of starting micropatterns (white dashed ellipse). One-way ANOVA with Tukey's test for multiple comparisons. Shaded bands and error bars +/−1 SD. Scale bar, 2 mm. Note, Angiostatin and Thalidomide were tested together and share the same Control. (K-M) Thalidomide, a known teratogen, disrupted and significantly decreased cVO EC formation over 16 days at 8 ug/mL (n=5, *p<0.01) and 80 ug/mL (n=6, ***p<0.0005) compared to 0 ug/mL (Control) (n=10). There was no significant (ns) difference between 8 and 80 ug/mL. At day 16 and 8 ug/mL, ECs congregated in the center area of starting micropatterns (white dashed circle) while at 80 ug/mL, ECs congregated at the periphery of starting micropatterns (white dashed ellipse). One-way ANOVA with Tukey's test for multiple comparisons. Shaded bands and error bars +/−1 SD. Scale bar, 2 mm. (N-O) Fentanyl, a potent opioid agonist, significantly increased cVO EC formation over 16 days at 10 nM (n=18, **p<0.001) compared to 0 nM (Control) (n=18). Student's t-test. Error bars +/−1 SD. Scale bar, 2 mm. (P-Q) Multiple linear regression model effects of DAPT (between 0 and 10 uM) and Dorsomorphin (between 0 and 1 uM) on cVO EC formation over 16 days (left). Multiple linear regression coefficients (middle), and mean R\hat{ }2 (right).

FIGS. 7A-7Q. Vascularization factors used for creating cVOs enable hVO creation. (A) Strategy for co-differentiating HCs, ECs, and SMCs to create hVOs. Key steps are inducing mesendoderm and then co-differentiating a vascular (CV) progenitor pool and hepatoblast pool that give rise to all three cell types. HC, hepatic cells. (B) Schematic showing timeline, stage, cell types, geometry, media, and growth factors for creating HCs, ECs, SMCs, and resulting hVOs. (C) Growth factor and small molecule characteristics for differentiation into HCs, ECs, and SMCs. Select references used as guidelines for concentrations and timing used in differentiation conditions are listed. Detailed conditions are listed in Table S2. (D) PCA of temporal RNA-seq shows developmental differences of hESC-3R (day 0 starting undifferentiated hESC-3R micropatterns) (green, n=3), Control (day 20 baseline hepatic differentiation, with no vascularization factors added) (red, n=3), hVO-D3 (day 20 hVOs created by adding vascularization factors at day 3 of differentiation) (cyan, n=3), compared to hVO-D6 (day 20 hVOs created by adding vascularization factors at day 6 of differentiation) (purple, n=3). Note divergence between “Vascularization Factors” (D3 and D6) and “No Vascularization Factors” (Control) groups. (E) RNA-seq WGCNA heat map showing clusters of Pluripotency, Structural, Miscellaneous Hepatic, Hepatic/Precursor, Hepatic and Smooth Muscle Cell, and Vascularization Genes over twenty days (D0-20) for hESC-3R (blue, n=3), Control (black, n=3), hVO-D3 (orange, D3), and hVO-D6 (red, n=3) groups. Black dashed rectangle shows vascularization genes are most upregulated in the hVO-D3 group. (F-M) Comparison between hESC-3R, Control, hVO-D3, and hVO-D6 for select gene groups: Pluripotent, Hepatic, Endothelial, Smooth Muscle Cell, Fibroblast, Extracellular Matrix, and Notch-Delta-Jag. Overall, pluripotent genes are upregulated for hESC-3R and downregulated for Control, hVO-D3, and hVO-D6 groups. Several hepatic genes are upregulated for Control, hVO-D3, and hVO-D6 groups. Notably, all EC, SMC, ECM, and Notch-Delta-Jag (except DLL3 and JAG1) genes are most upregulated for the hVO-D3 group. One-way ANOVA with Tukey's test for multiple comparisons, *p<0.05, **p<0.01, ***p<0.001, ****p<0.0001, ns, not significant. (N-P) Over 19 days, ECs increase most for the hVO-D3 group (n=12) (dashed orange square in (N)) compared to the hVO-D6 (n=10) and Control (n=10) groups, indicating that the most ECs form when vascular factors are added at day 3 to the baseline hepatic differentiation protocol. One-way ANOVA with Tukey's test for multiple comparisons, **p<0.001, ***p<0.0001. Shaded bands and error bars +/−1 SD. Scale bar, 2 mm. (Q) Individual confocal fluorescence images showing HCs (AFP, white) (left), ECs (CDH5-mOr, orange) (middle), and merge (right). Nuclei are labeled with DAPI (blue). Scale bar, 2 mm.

FIG. 8. Micropatterning of hPSCs results in spatially organized cardiomyocytes, related to FIG. 1. Phase images showing reproducible 2 mm (left), 4 mm (middle), and 6 mm (right) undifferentiated hPSC micropatterns in 16 wells of a 48-well plate. Representative micropatterns are outlined by white dashed circles. Scale bar, 10 mm.

FIGS. 9A-9F. Micropatterning gives rise to organized germ layer formation, related to FIGS. 2 and 3. (A) The hESC-RUES-GLR (germ layer reporter) cell line (Martyn et al., 2018) used to visualize germ layer formation comprises BRA driving mCerulean expression to visualize mesoderm formation, SOX17 driving tdTomato expression to visualize early endoderm formation (and late arterial formation), and SOX2 driving mCitrine to visualize ectoderm formation and pluripotency. (B) Fluorescence images showing mesendoderm differentiation of a 2 mm hPSC micropattern at day 3 with circular distribution (white circular dashes) of BRA+ (mesoderm, red) and SOX17+ (endoderm, green). Scale bar 2 mm. (C) Phase and fluorescence images showing temporal expression from days 0 to day 11 (D0-11) of the mesodermal marker BRA and the endodermal/arterial marker SOX17 of micropatterned arrays of RUES-GLR hESCs. Scale bar, 2 mm. (D) Enlarged view of dashed square in (A). Note BRA+ central distributed expression relative to outer SOX17+ expression. Scale bar, 2 mm. (E) Fluorescence images showing that hESC-3R micropatterns (described in detail in FIG. 3 and S3) treated with CHIR+FGF2+IWR (Control) differentiated to the most CMs (green) at day 14 with a CHIR concentration of 5 uM. ECs (orange) are present at all concentrations but are punctate, with minimal branching. (F) Fluorescence images showing that hESC-3R micropatterns treated with CHIR+FGF2+IWR+VEGF (+VEGF at days 5, 7, 9, and 11) co-differentiated to the most CMs (green) and ECs (orange) at day 14 with a CHIR concentration of 5 uM and VEGF concentration of 50 ng/ml.

FIG. 10A-10P Creation of a triple-reporter line enables screening of differentiation conditions leading to cVO formation, related FIG. 3. (A) Strategy for creating the lentivector pLV-CDH5Pr-mOrange-SVPr-Zeo used to create the hESC-3R line. The CDH5 (VE-Cadherin) promoter drives mOrange to identify ECs. (B) Plasmid map of pLV-CDH5Pr-mOrange-SVPr-Zeo. (C) Strategy for creating the lentivector pLV-TAGLNPr-CFP-SVPr-Bsd used to create the hESC-3R line. The TAGLN (SM22a) promoter drives CFP to identify SMCs. (D) Plasmid map of pLV-TAGLNPr-CFP-SVPr-Bsd. (E) Gel showing genomic PCR products of two polyclones of the hESC-3R line (hESC^{TNNT2-GFP/CDH5-mOrange/TAGLN-CFP} −1 and −2). A (DNA) Ladder, GAPDH (housekeeping gene), CFP, Blasticidin, mOrange, CDH5, Zeocin1, Zeocin2, and GFP are shown. (F) Gel showing genomic PCR products of six monoclones (C1-C6) of the hESC-3R line. C3 and C5 (red rectangles) show the highest signals for all products. A (DNA) Ladder, GAPDH (housekeeping gene), CFP, Blasticidin, mOrange, CDH5, Zeocin1, Zeocin2, and GFP are shown. (G) Gel showing genomic PCR products of a monoclonal hESC-3R line. A (DNA) Ladder, non-template (NT) control, GAPDH (housekeeping gene), POU5F1, CFP, Blasticidin, mOrange, and Zeocin1 are shown. (H) Strategy for co-differentiating CMs, ECs, and SMCs to create cVOs. A key step is creating a cardiovascular (CV) progenitor pool that gives rise to all three cell types. (I) Growth factor and small molecule characteristics for differentiation into CMs, ECs, and SMC. Select references are listed that were used as guidelines for concentrations and timing used in the screening conditions. Detailed screening conditions are listed in Table 1. (J) Phase and fluorescence images showing time-course formation from days 0 to 10 of CMs, ECs, and SMCs from hESC-3R micropatterned arrays. Condition 2 (Control) using CHIR 5 uM gives rise to CMs, some ECs, and no SMCs. Condition 31 using the same vascular factors as Condition 32, but with CHIR 4 um gives rise to ECs and SMCs, but no CMs, while Condition 32 gives rise to all three cell types. Scale bar, 2 mm. (K) A total of thirty-four (34) screening conditions (n=175 total micropatterns) were used to create various combinations of CMs, ECs, and SMCs. Odd numbered conditions (1-33) used CHIR 4 uM (n=88 micropatterns, gray) and even numbered conditions (2-34) used 5 uM (n=87 micropatterns, purple). Overall, 5 uM gave rise to the most CMs, ECs, and SMCs compared to 4 uM for each condition. (L) Conditions using CHIR 4 (grey) or 5 uM (green) (n=4-6 per condition) showing largest CM formation (GFP total fluorescence area) at day 12 for

Condition 32 (green arrow). Overall, 5 uM gave rise to the most CMs compared to 4 uM for each condition. Error bars +/−1 SD. (M) Conditions using CHIR 4 (grey) or 5 uM (orange) (n=4-6 per condition) showing largest EC formation (mOr total fluorescence area) at day 12 for Condition 32 (orange arrow). Overall, 5 uM gave rise to the most ECs compared to 4 uM for each condition. Error bars +/−1 SD. (N) Conditions using CHIR 4 (grey) or 5 uM (blue) (n=4-6 per condition) showing largest SMC formation (CFP total fluorescence area) at day 12 for Condition 32 (blue arrow). Overall, 5 uM gave rise to the most SMCs compared to 4 uM for each condition. Error bars +/−1 SD. (O) A total of thirty-four (34) screening conditions showing total fluorescence area for CMs (GFP), ECs (mOr), and SMCs (CFP) from days 0-12 for each condition. Overall, Condition 32 (black solid arrow) gave rise to the most CMs, ECs, and SMCs. Condition 2 (black dashed arrow) is the standard Control CM differentiation with only the small molecules CHIR, FGF2, and IWR. Error bars +/−1 SD. (P) Time-course CM formation (GFP total fluorescence area) and EC formation (mOr total fluorescence area) for Conditions 32 (n=6). The rate of CM formation (dashed green line) increases around day 8 while the rate of EC formation (dashed orange line) increases around day 5. Note EC formation is already apparent at day 3. Shaded bands +/−1 SD.

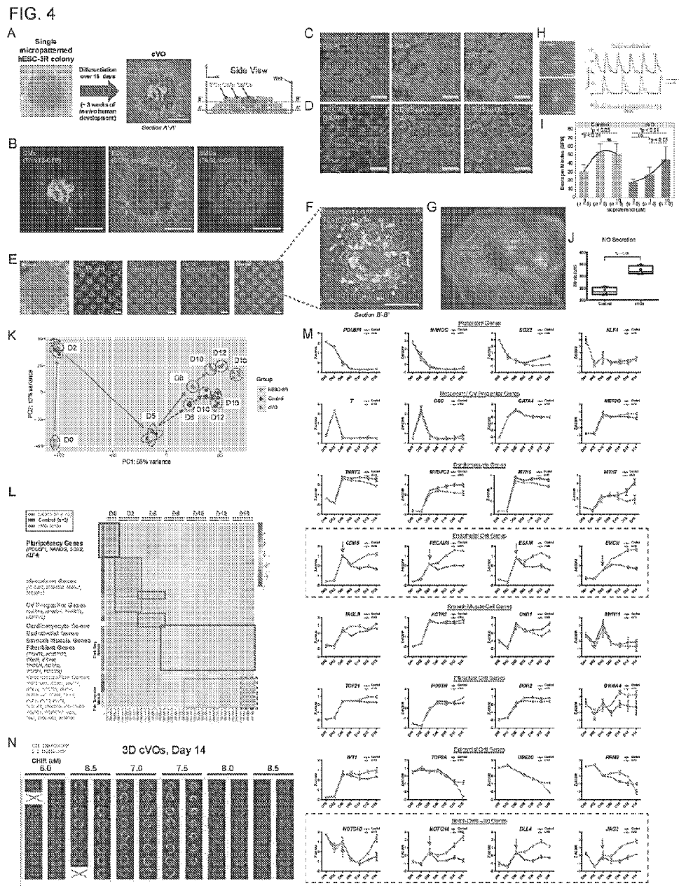

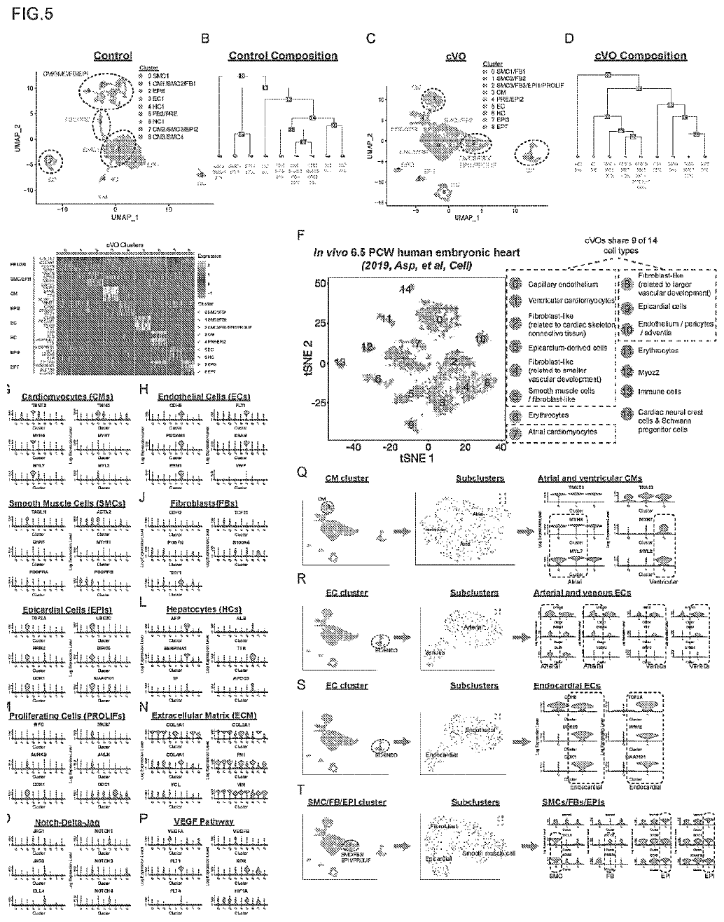

FIGS. 11A-11S. cVOs comprise spatially and temporally self-organized cardiovascular cell types, related to FIG. 4. (A) Phase and fluorescence images showing the differentiation of unpatterned (top row) and micropatterned (bottom row) hESC-3R cells over 16 days containing CMs (TNNT2-GFP, green) and ECs (CDH5-mOr, orange). Note the unorganized distribution of CMs and ECs in the unpatterned group. Scale bar, 2 mm. (B) An enlarged inset from the black rectangle in (A) showing CMs, ECs, and SMCs. White arrowheads show ECs surrounding a group of CMs. Scale bar, 500 um. (C) Maximum intensity image of ECs in (B) shows branching. Scale bar, 500 um. (D) Representative contraction-relaxation cycle of a single cVO (shown in the three images above each phase, with maximum contraction and maximum relaxation shown in red). (E) The mean beating rate of Controls (n=4) was higher than cVOs (n=4), but not statistically significant (p=0.65). Student's t-test, p<0.05 considered significant. (F) The mean contraction velocity of Controls (n=4) was higher than cVOs (n=4), but not statistically significant (p=0.06). Student's t-test, p<0.05 considered significant. (G) The mean relaxation velocity of Controls (n =4) was higher than cVOs (n=4), but not statistically significant (p=0.10). Student's t-test, p<0.05 considered significant. (H) The mean contraction-relaxation peak interval of Controls (n=4) was lower than cVOs (n=4), but not statistically significant (p=0.52). Student's t-test, p<0.05 considered significant. (I) Day 16 confocal fluorescence images showing a micropatterned hiPSC-derived cVO immunostained for the CM marker Troponin-T (TnT), the EC marker VE-Cadherin (VECad), and the SMC marker smooth muscle 22° (SM22α). Nuclei are labeled with DAPI (blue). Scale bar, 1 mm. (J) Ingenuity Pathway Analysis (IPA) of the temporal bulk-RNA-sequencing (RNA-seq) weighted gene co-expression network analysis (WGCNA) Pale Turquoise module within the “Vascularization Genes” cluster in FIG. 4L confirming a vascularization regulator effect network with upstream regulators including VEGF, CD36, JAG2, and JUNB activating genes including PDGFB, FLT1, MMP9, TGFβ1, HIF1A, FN1, and DLL4, leading to downstream vascularization functions. Green indicates measured upregulated genes (|log2FC|>2). (K) IPA of the temporal RNA-seq WGCNA Dark Grey module within the “Cardiomyocyte/Endothelial/Smooth Muscle/Fibroblast Genes” cluster in FIG. 4L confirming a cardiogenesis regulator effect network with upstream regulators including BMP, TGFβ, and WNT11 activating genes including MEF2C, NKX2-5, TBX5, MYH6, and NPPA leading to downstream cardiogenesis. Green indicates measured upregulated genes (|log2FC|>2)). (L) Days 2 to 16 cVO compared to Control IPA showing a heat map of upregulated (red) and downregulated (blue) upstream regulators (including PDGFBB, TGF [symbol] 1, CD36, BMP4, VEGFA, FGF2, and HIF1A). (M) Days 2 to 16 cVO compared to Control IPA showing a heat map of upregulated (red) and downregulated (blue) canonical pathways. (N) Days 2 to 16 cVO compared to Control IPA showing a heat map of upregulated (red) and downregulated (blue) vascularization functions. (O) Day 16 cVO compared to Control IPA showing a heat map of vascularization functions. Functions>Z-score of 2 are shaded red. Size of rectangles are proportional to -log (p-value). (P) Day 16 cVO compared to Control IPA showing significantly activated vascularization functions (-log (p-value)>1.3). (Q) Day 16 cVO compared to Control IPA showing VEGF regulator effect network on ITGA1, MMP9, CDH5, F3, NOTCH4, CD34, and DLL4 leading to downstream development of vasculature. Green indicates measured upregulated genes (|log2FC|>2). (R) Ingenuity pathway analysis (IPA) shows genes of the NOTCH pathway for the Control group at day 16 of differentiation (compared to day 0) are downregulated (red). In contrast, genes of the NOTCH pathway for the cVO group at day 16 of differentiation (compared to day 0) are upregulated (green). (S) IPA shows most genes are upregulated (green) in the BMP pathway for both the Control and cVO groups at day 16 of differentiation (compared to day 0). BMP-2 is more upregulated in cVOs compared to Controls.

FIGS. 12A-12D. scRNA-seq reveals multiple vascular, endocardial, myocardial, and epicardial cell types in cVOs, related to FIG. 5. (A) Control UMAP feature plots showing CM (TNNT2, TNNI3, MYH6, MYH7), EC (CDH5, FLT1, PECAM1, ESAM), SMCs (TAGLN, ACTA2, CNN1, MYH11), FB (DDR2, TCF21, POSTN, S100A4), EPI (TOP2A, UBE2C, RRM2, BIRC5), HC (AFP, ALB, SERPINA1, TTR), PROLIF (MYC, MK167, AURKB, ANLN), ECM (COL1A1, COL3A1, COL4A1, FN1), and NOTCH-DELTA-JAG pathway (NOTCH1, NOTCH2, NOTCH3, NOTCH4, JAG1, JAG2, DLL3, DLL4) gene expression. (B) Control violin plots showing cardiovascular progenitor (CV PROG), gap junction (GJ), EC, ECM, WNT pathway, BMP pathway, PDGF pathway, and TGFβ pathway gene expression. (C) cVO UMAP feature plots showing CM (TNNT2, TNNI3, MYH6, MYH7), EC (CDH5, FLT1, PECAM1, ESAM), SMCs (TAGLN, ACTA2, CNN1, MYH11), FB (DDR2, TCF21, POSTN, S100A4), EPI (TOP2A, UBE2C, RRM2, BIRC5), HC (AFP, ALB, SERPINA1, TTR), PROLIF (MYC, MKI67, AURKB, ANLN), ECM (COL1A1, COL3A1, COL4A1, FN1), and NOTCH-DELTA-JAG pathway (NOTCH1, NOTCH2, NOTCH3, NOTCH4, JAG1, JAG2, DLL3, DLL4) gene expression. (D) cVO violin plots showing cardiovascular progenitor (CV PROG), gap junction (GJ), EC, ECM, WNT pathway, BMP pathway, PDGF pathway, and TGFβ pathway gene expression.

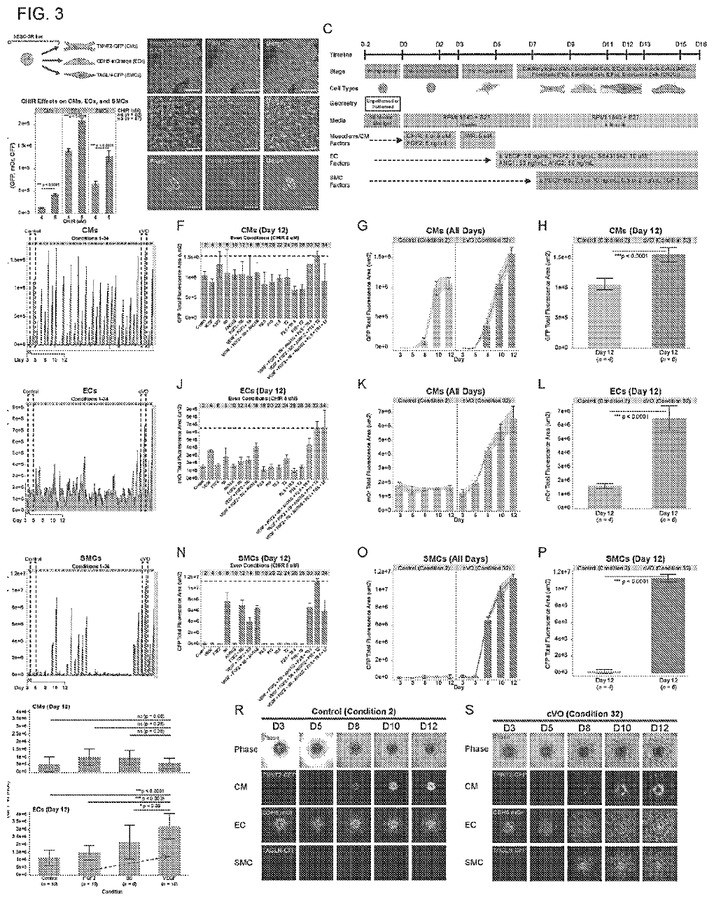

FIGS. 13A-13B. A machine learning multiple linear regression model created from screening conditions for cVO formation, related to FIG. 3. (A) Multiple linear regression model created from 34 screening conditions (C1-C34) for cVO formation showing TNNT2-GFP total fluorescence area from days 2 to 16, indicating CM formation over time. Grey lines indicate 5-6 estimates from the original data, green lines indicate the mean of the estimates, and black lines indicate the fit of the model. The solid green square identifies C2 as the Control condition, and the dashed green square identifies C32 as the condition resulting in CMs within cVOs. (B) Multiple linear regression model created from 34 screening conditions (C1-C34) for cVO formation showing CDH5-mOrange total fluorescence area from days 2 to 16, indicating EC formation over time. Grey lines indicate 5-6 estimates from the original data, green lines indicate the mean of the estimates, and black lines indicate the fit of the model. The solid orange square identifies C2 as the Control condition, and the dashed orange square identifies C32 as the condition resulting in ECs within cVOs.

FIGS. 14A-14M. Temporal bRNA-seq expression for various cVO genes, related to FIG. 4. (A) Mesoderm-precursor genes. (B) Atrial genes. (C) Ventricular genes. (D) Adrenergic genes. (E) Endothelial/arterial/venous/endocardial genes. (F) BMP pathway genes. (G) TGF [symbol] pathway genes. (H) VEGF pathway genes. (I) WNT pathway genes. (J) PDGF pathway genes. (K) Angiopoietin pathway genes. (L) Gap junction genes. (M) Paracrine signaling genes.

FIG. 15. Flow chart for differentiation to create cardiac vascularized organoids (CVO).

FIG. 16. Flow chart for differentiation to create hepatic vascularized organoids (HVO).

FIG. 17. Flow chart for differentiation to create neural vascularized organoids (NVO).

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those having skeletal muscle dysfunction or deficiency, brown fat dysfunction or deficiency, dorsal dermis dysfunction or deficiency, cartilage dysfunction or deficiency, bone dysfunction or deficiency, smooth muscle dysfunction or deficiency, cardiomyocyte dysfunction or deficiency, etc.) as well as those in which prevention is desired (e.g., those with increased susceptibility to a dysfunction or deficiency; those suspected of having a dysfunction or deficiency; those having one or more risk factors, etc.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “pluripotent progenitor cells”, “pluripotent progenitors”, “pluripotent stem cells”, “multipotent progenitor cells” and the like, as used herein refer to cells that are capable of differentiating into two or more different cell types and proliferating. Non limiting examples of pluripotent precursor cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like. Pluripotent progenitor cells may be acquired from public or commercial sources or may be newly derived.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 10¹⁰ or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

The terms “homogenous population”, as it relates to cell populations, refers to a cell population that is essentially pure and does not consist of a significant amount of undesired or contaminating cell types. By significant amount, in this context, is meant an amount of undesired or contaminating cell types that negatively impacts the use of the isolated desired cell population. As such, the actual amount of undesired or contaminating cells that defines a significant amount will vary and depend on the particular type of undesired or contaminating cells and/or the particular use of the desired cell type. For example, in a population of differentiated mesodermal cells used in the treatment of a subject, a significant amount of improperly differentiated contaminating cell types will be small as such cells may have a high capacity to negatively impact the use of the generated desired cell population. In comparison, e.g., in a population of differentiated mesodermal cells used in the treatment of a subject, a significant amount of contaminating progenitor cells may be relatively large as such cells may have a low capacity to negatively impact the use of the generated desired cell population. In some instances, a homogenous population may refer to a highly enriched population. Levels of homogeneity will vary, as described, and may, in some instances, be greater than 60% pure, including e.g., more than 65%, more than 70%, more than 75%, more than 80%, more than 85%, more than 90%, more than 95%, more than 96%, more than 97%,. more than 98%, more than 99%, more than 99.5%, more than 99.6%, more than 99.7%, more than 99.8%, and more than 99.9%.

The term “heterologous”, as it refers to a “heterologous sequence” or “heterologous nucleic acid”, means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter.

### Methods of the Invention

Methods are provided for the obtention and use of in vitro cell cultures of vascularized organoids, comprising spatially organized and branched vascular network within endocardial, myocardial, epicardial, and/or progenitor cells. In some embodiments of the methods to generate vascularized organoids, geometrically micropatterned pluripotent stem cells (PSCs), e.g. human PSCs, such as human embryonic stem cells (hESCs) or human induced pluripotent stem cells (hiPSCs), are used to initiate a culture. The PSCs are subsequently differentiated into germ layers, progenitor cell types, and differentiated cell types using biochemical growth factors and small molecules.

Pluripotent stem cells may be acquired from any convenient source, including but not limited to newly derived from a subject of interest or tissue specimen or other cellular sample, obtained from a public repository, obtained from a commercial vendor, and the like. In some instances, pluripotent cells of interest include human cells including but not limited to, e.g., human embryonic stem cells, human induced pluripotent stem cells, human fetal stem cells, and the like.

In some instances, pluripotent progenitor cells of the subject disclosure may be unmodified such that the cells have not been genetically or otherwise modified from their natural state prior to culture according to the methods described herein. In other instances, pluripotent progenitor cells of the subject disclosure may be modified such that the cells are genetically or otherwise modified from their natural state. For example, the PSC may be genetically modified to comprise one or more reporter genes, e.g. one, two, three, four or more fluorescent reporters, which reporters enable temporal identification of germ layer, progenitor, and/or cardiovascular cell types in situ without disturbing the architecture of developing VOs.

Genes may be introduced into the PSC for a variety of purposes, e.g. to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy PSC through targeted genetic manipulation (CRISPR/CAS9, etc.) or PSC can be derived from individual patients that carry a disease-related genotype or are diagnosed with a disease and “corrected” to healthy PSC, also through targeted genetic manipulation (CRISPR/CAS9, etc.). Conditions of various diseases that have strong genetic components or are directly caused by genetic or genomic alterations can be modeled with the systems of the invention.

In some embodiments a panel of such VOs are provided, where the panel includes two or more different disease-relevant VOs. In some embodiments a panel of such VOs are provided, where the VOs are subjected to a plurality of candidate agents, or a plurality of doses of a candidate agent. Candidate agents include small molecules, i.e. drugs, genetic constructs that increase or decrease expression of an RNA of interest, electrical changes, and the like. In some embodiments a panel refers to a system or method utilizing patient-specific cells from two or more distinct disease conditions, and may be three or more, four or more, five or more, six or more, seven or more distinct conditions.

In one embodiment, cultures are initiated with PSCs micropatterned on the surface of a culture vessel with a suitable matrix material. A suitable matrix includes Matrigel, Cultrex, Geltrex, etc. The primary components of Matrigel are four major basement membrane ECM proteins: laminin, collagen IV, entactin and the heparin sulfate proteoglycan perlecan. Synthetic alternatives to Matrigel are known in the art, for a review see Aisenbrey and Murphy (2020) Nat Rev Mater. 5(7):539-551, herein specifically incorporated by reference.

Micropatterning in culture may comprise introducing a stencil or equivalent to a culture well, wherein the stencil comprises holes of from about 1 mm, about 2 mm, about 3 mm, up to about 10 mm diameter or an equivalent size in a non-circular pattern, up to about 10 mm. A suitable matrix is poured over the stencil, and the PSC are allowed to attach to the matrix. After removal of the stencil, micropatterned cells are attached on the islands created by the holes of the stencils. In alternative embodiments, other suitable means can be used to pattern a matrix on the surface of a culture vessel, i.e. dispensing a matrix solution from a printer, and the like, preprinting surfaces, etc. where the hPSC are then allowed to adhere. The matrix may be provided in any convenient geometry, e.g. a circle, oval, square, rectangle, etc. When the hPSC become confluent, e.g., after from about 1 to 5 days, additional matrix medium can be added to allow the colonies to migrate beyond the initial boundaries.

The micropatterned colonies are co-differentiated into cells of interest using biochemical growth factors and small molecules suitable for the desired cell types, utilizing a series of steps specific media for in vitro differentiation. The differentiation protocol allows for formation of the three primary germ layers, comprising the endoderm, mesoderm, and ectoderm, and subsequent differentiation to create vascularized organoids. By controlling the ratio of each of the germ layers through simultaneous induction of the desired germ layers, vascularized structures that derive from the germ layers are generated. Exemplary flow charts for differentiation are provided in FIGS. 15, 16 and 17.

The specific combination of factors and steps for differentiation will be selected based on the desired end product. Provided below are exemplary differentiation schemes.

Cardiac Vascularized Organoid (cVO) Differentiation. (step 1) PSCs in single cell suspension are transferred to micropatterned surfaces as described above, and differentiated in suitable medium, e.g., RPMI, supplemented with B27 without insulin. On Day 0, for mesoderm induction, basal medium is supplemented with an effective dose of a WNT pathway activator and an effective dose of an activator of the FGF pathway agent for a period of from about 24 to about 72 hours, and may be not more than about 48 hours. In some embodiments the WNT pathway activator is CHIR99021 (CHIR). The effective dose may be or may have activity equivalent to from about 1-20 μM CHIR, from about 3-10 μM, from about 4-8 μM. In some embodiments the activator of the FGF pathway is FGF-2. The effective dose may be from about 1 ng/ml to about 20 ng/ml, from about 5 ng/ml to about 10 ng/ml.

Following mesoderm induction, the cells are (step 2) cultured to generate cardiomyocytes, in the presence of an effective concentration of a Wnt inhibitor, including without limitation IWR-1, where the effective dose may be or may have activity equivalent to from about 0.1 μM to about 100 μM, and may be from about 1 μM to about 10 μM, for a period of from about 24 to about 72 hours, and may be about 48 hours.

Subsequently, on about day 4, about day 5, about day 6, to about day 15, day 16, day 17, the cells are (step 3) cultured to simultaneously induce endothelial cell vasculogenesis and angiogenesis along with the cardiomyocyte differentiation by culture in the presence of an effective dose of a VEGF agonist; an effective dose of an activator of the FGF pathway, an effective dose of an inhibitor of the SMAD pathway; an effective dose of an angiopoietin activating agent. In some embodiments the VEGF agonist is a VEGF protein, e.g. VEGF-165. The effective dose may be or may have activity equivalent to from about 5 ng/ml to about 100 ng/ml, from about 10 to about 75 ng/ml, and may be about 50 ng/ml. In some embodiments the activator of the FGF pathway is FGF-2. The effective dose may be or may have activity equivalent to from about 1 ng/ml to about 20 ng/ml, from about 5 ng/ml to about 10 ng/ml of FGF2. In some embodiments the inhibitor of the SMAD pathway is SB431542. The effective dose may be or may have activity equivalent to from about 1 μM to about 50 μM, from about 2.5 μM to about 25 μM, from about 5 μM to about 10 μM SB431542. In some embodiments the angiopoietin activating agent is Angiopoietin-2 (ANG2), or a combination of Angiopoietin-1 and-2. The effective dose may be or may have activity equivalent to from about 5 to about 100 ng/ml, from about 10 to about 75 ng/ml, and may be about 50 ng/ml ANG2 or ANG1+ANG2. Additional agents may include 5 ng/ml EGF, 15 ng/mL IGF-1, 50 μg/mL ascorbic acid, 0.75 U/mL heparin sulfate, 1 μg/mL hydrocortisone.

On about day 5, day 6, day 7, to about day 15, day 16, day 17, the cells are (step 4) cultured to simultaneously induce smooth muscle cell differentiation with cardiomyocyte and endothelial cell differentiation, by culture in the presence of an effective dose of a PDGF pathway activating agent; and a TGF-β1 activating agent. In some embodiments the PDGF pathway activating agent is PDGF-BB. The effective dose may be or may have activity equivalent to from about 1 ng/ml to about 25 ng/ml, from about 5 ng/ml to about 10 ng/ml of PDGF-BB. In some embodiments the TGF-β1 activating agent is TGF-β1. The effective dose may be or may have activity equivalent to from about 1 ng/ml to about 25 ng/ml, from about 5 ng/ml to about 10 ng/ml of TGF-β1.

For three-dimensional cVOs, the initial micropatterning of a matrix may be performed on a surface with a hydrogel surface. The stiffness of the hydrogel may be selected for the desired cell types. Alternatively, for micropatterning, each culture well may be coated with a central droplet of matrix.

Hepatic Vascularized Organoid (hVO) Differentiation. (step 1) PSCs in single cell suspension are transferred to micropatterned surfaces as described above, and differentiated in suitable medium, e.g. basal medium, for mesendodermal induction, in the presence of an effective dose of an activator of TGF-β pathway, e.g. Activin-A (ActA); an effective dose of an activator of BMP signaling pathway; an effective dose of a WNT pathway activating agent; an effective dose of a PI3K pathway inhibitor. In some embodiments the activator of TGF-β pathway is Activin-A (ActA). The effective dose may be or may have activity equivalent to from about 10 ng/ml to about 250 ng/ml, from about 50 ng/ml to about 100 ng/ml of ActA. In some embodiments the activator of BMP signaling is BMP-4 (BMP4). The effective dose may be or may have activity equivalent to from about 1 ng/ml to about 25 ng/ml, from about 5 ng/ml to about 10 ng/ml of BMP4. In some embodiments the WNT pathway activator is CHIR99021 (CHIR). The effective dose may be or may have activity equivalent to from about 1-20 μM CHIR, from about 3-10 μM, from about 4-8 μM. In some embodiments the activator of the FGF pathway is FGF-2. The effective dose may be from about 1 ng/ml to about 20 ng/ml, from about 5 ng/ml to about 10 ng/ml. In some embodiments the PI3K pathway inhibitor is LY294002. The effective dose may be or may have activity equivalent to from about 1 μM to about 50 μM, from about 2.5 μM to about 25 μM, from about 5 μM to about 10 μM LY294002.

Following mesendoderm induction, the cells are (step 2) cultured for foregut induction, in the presence of an effective concentration of an FGF activator, including without limitation FGF10. The effective dose may be or may have activity equivalent to from about 10 ng/ml to about 250 ng/ml, from about 50 ng/ml to about 100 ng/ml of FGF10, for a period of from about 24 to about 72 hours, and may be about 48 hours.

The cells are then (step 3) on about day 4, day 5, day 6, cultured for hepatoblast induction, in the presence of an effective concentration of an FGF activator; and an effective dose of an activator of BMP signaling pathway. In some embodiments the activator of BMP signaling is BMP-4 (BMP4). The effective dose may be or may have activity equivalent to from about 1 ng/ml to about 25 ng/ml, from about 5 ng/ml to about 10 ng/ml of BMP4. The FGF activator may be FGF10, where the effective dose may be or may have activity equivalent to from about 10 ng/ml to about 250 ng/ml, from about 50 ng/ml to about 100 ng/ml of FGF10, for a period of from about 24 to about 72 hours, and may be about 48 hours.

For hepatocyte induction, after about day 6, the cells are (step 4) cultured in the presence of an effective concentration of Hepatocyte Growth Factor (HGF), an effective concentration of Oncostatin-M (OncoM), and an effective concentration of dexamethasone. The effective concentration of HGF may be or may have activity equivalent to from about 10 ng/ml to about 250 ng/ml, from about 50 ng/ml to about 100 ng/ml of HGF. The effective concentration of OncoM may be or may have activity equivalent to from about 10 ng/ml to about 250 ng/ml, from about 50 ng/ml to about 100 ng/ml of OncoM. The effective concentration of DEX may be e or may have activity equivalent to from about 1 μM to about 50 μM, from about 2.5 μM to about 25 μM, from about 5 μM to about 10 μM DEX.

To simultaneously induce endothelial cell vasculogenesis and angiogenesis along with the hepatocyte differentiation the cells are cultured (step 5) from about day 3, day 4, day 6 to about day 15, day 18, day 20 in the presence of an effective dose of a VEGF agonist; an effective dose of an activator of the FGF pathway, an effective dose of an inhibitor of the SMAD pathway; an effective dose of an angiopoietin activating agent. In some embodiments the VEGF agonist is a VEGF protein, e.g., VEGF-165. The effective dose may be or may have activity equivalent to from about 5 ng/ml to about 100 ng/ml, from about 10 to about 75 ng/ml, and may be about 50 ng/ml. In some embodiments the activator of the FGF pathway is FGF-2. The effective dose may be or may have activity equivalent to from about 1 ng/ml to about 20 ng/ml, from about 5 ng/ml to about 10 ng/ml of FGF2. In some embodiments the inhibitor of the SMAD pathway is SB431542. The effective dose may be or may have activity equivalent to from about 1 μM to about 50 μM, from about 2.5 μM to about 25 μM, from about 5 μM to about 10 μM SB431542. In some embodiments the angiopoietin activating agent is Angiopoietin-2 (ANG2), or a combination of Angiopoietin-1and -2. The effective dose may be or may have activity equivalent to from about 5 to about 100ng/ml, from about 10 to about 75 ng/ml, and may be about 50 ng/ml ANG2 or ANG1+ANG2. Additional agents may include 5 ng/ml EGF, 15 ng/ml IGF-1, 50 μg/mL ascorbic acid, 0.75 U/mL heparin sulfate, 1 g/mL hydrocortisone.

To simultaneously induce smooth muscle cell differentiation with hepatocyte and endothelial cell differentiation, on about day 5, day 6, day 7, to about day 15, day 16, day 17, the cells are (step 6) cultured in the presence of an effective dose of a PDGF pathway activating agent; and a TGF-β1 activating agent. In some embodiments the PDGF pathway activating agent is PDGF-BB. The effective dose may be or may have activity equivalent to from about 1 ng/ml to about 25 ng/ml, from about 5 ng/ml to about 10 ng/ml of PDGF-BB. In some embodiments the TGF-β1 activating agent is TGF-β1. The effective dose may be or may have activity equivalent to from about 0.1 ng/ml to about 5 ng/ml, from about 0.5 ng/ml to about 1 ng/ml of TGF-β1.

Neural Vascularized Organoid (nVO) Differentiation. (step 1) PSCs in single cell suspension are transferred to micropatterned surfaces as described above, and differentiated in suitable medium, e.g., basal medium, for induction, in the presence of an effective dose of a WNT pathway activating agent; an effective dose of a SMAD pathway inhibitor; an effective dose of dorsomorphin (DM). In some embodiments the inhibitor of the SMAD pathway is SB431542. The effective dose may be or may have activity equivalent to from about 1 μM to about 50 μM, from about 2.5 μM to about 25 μM, from about 5 μM to about 10 μM SB431542. In some embodiments the WNT pathway activator is CHIR99021 (CHIR). The effective dose may be or may have activity equivalent to from about 1-20 μM CHIR, from about 3-10 μM, from about 4-8 μM. The effective dose of DM is or may have activity equivalent to from about 1-20 μM DM, from about 3-10 μM, from about 4-8 μM.

From about day 5 to about day 11, (step 2) the cells are cultured in the presence of an effective dose of a WNT pathway activating agent; an effective dose of a SMAD pathway inhibitor; an effective concentration of LIF Interleukin 6 Family Cytokine and an effective concentration of a basic FGF activating agent. In some embodiments the inhibitor of the SMAD pathway is SB431542. The effective dose may be or may have activity equivalent to from about 1 μM to about 50 μM, from about 2.5 μM to about 25 μM, from about 5 μM to about 10 μM SB431542. In some embodiments the WNT pathway activator is CHIR99021 (CHIR). The effective dose may be or may have activity equivalent to from about 1-20 μM CHIR, from about 3-10 μM, from about 4-8 μM. In some embodiments the effective dose of LIF is or may have activity equivalent to from about 1 ng/ml to about 20 ng/ml, from about 5 ng/ml to about 10 ng/ml of LIF. In some embodiments the bFGF activating factor is bFGF protein. The effective concentration may be or may have activity equivalent to from about 1 ng/ml to about 20 ng/ml, from about 5 ng/ml to about 10 ng/ml of bFGF.

To simultaneously induce endothelial cell vasculogenesis and angiogenesis along with the neural differentiation the cells are cultured from about day 3, day 4, day 6 to about day 15, day 18, day 20 in the presence of an effective dose of a VEGF agonist; and an effective dose of an angiopoietin agent. In some embodiments the VEGF agonist is a VEGF protein, e.g., VEGF-165. The effective dose may be or may have activity equivalent to from about 5 ng/ml to about 100 ng/ml, from about 10 to about 75 ng/ml, and may be about 50 ng/ml. In some embodiments the angiopoietin activating agent is Angiopoietin-2 (ANG2), or a combination of Angiopoietin-1 and -2. The effective dose may be or may have activity equivalent to from about 5 to about 100 ng/ml, from about 10 to about 75 ng/ml, and may be about 50 ng/ml ANG2 or ANG1+ANG2. Additional agents may include 5 ng/mL EGF, 15 ng/ml IGF-1, 50 μg/mL ascorbic acid, 0.75 U/mL heparin sulfate, 1 μg/mL hydrocortisone.

From about day 7 the culture may additionally include a PDGF activating agent. In some embodiments the PDGF pathway activating agent is PDGF-BB. The effective dose may be or may have activity equivalent to from about 1 ng/ml to about 25 ng/ml, from about 5 ng/ml to about 10 ng/ml of PDGF-BB.

From about day 11 (step 3), the neural cells are induced by culture in the presence of an effective concentration a SMAD pathway inhibitor; an effective concentration of a basic FGF activating agent; an effective concentration of a brain derived neurotrophic factor (BDNF), an effective concentration of glial cell line-derived neurotrophic factor. In some embodiments the inhibitor of the SMAD pathway is SB431542. The effective dose may be or may have activity equivalent to from about 1 μM to about 50 μM, from about 2.5 μM to about 25 μM, from about 5 μM to about 10 μM SB431542. In some embodiments the bFGF activating factor is bFGF protein. The effective concentration may be or may have activity equivalent to from about 1 ng/ml to about 20 ng/ml, from about 5 ng/ml to about 10 ng/ml of bFGF. In some embodiments the GDNF factor is GDNF protein. The effective concentration may be or may have activity equivalent to from about 1 ng/ml to about 20 ng/ml, from about 5 ng/ml to about 10 ng/ml of GDNF. In some embodiments the BDNF factor is BDNF protein. The effective concentration may be or may have activity equivalent to from about 1 ng/ml to about 20 ng/ml, from about 5 ng/ml to about 10 ng/ml of BDNF.

Pluripotent progenitors and derivatives thereof may be contacted with differentiation factors by any convenient means. Generally, a differentiation factor is added to culture media, as described herein, within which cells of the instant disclosure are grown or maintained, such that the induction agent is present, in contact with the cells, at an effective concentration to produce the desired effect, e.g., induce a desired lineage restriction event. In other instances, e.g., where the existing culture media is not compatible with a particular induction agent, the culture media in which the cells are being grown is replaced with fresh culture media containing the particular induction agent present in the fresh media at an effective concentration to produce the desired effect. In instances where fresh or specific culture media is provided with a particular induction agent the culture agent may, in some instances, be specifically formulated for the particular induction agent, e.g., containing one or more specific additional reagents to, e.g., aid in the delivery of the induction agent, aid in the solubility of the induction agent, aid in the stability of the induction agent, etc.

The effective concentration of a particular differentiation factor will vary and will depend on the agent. In addition, in some instances, the effective concentration may also depend on the cells being induced, the culture condition of the cells, other induction agents co-present in the culture media, etc. As such, the effective concentration of induction agents will vary and may range from 1 ng/mL to 10 μg/mL or more, including but not limited to, e.g., 1 ng/ml, 2 ng/ml, 3 ng/ml, 4 ng/ml, 5 ng/ml, 6 ng/ml, 7 ng/ml, 8 ng/ml, 9 ng/mL, 10 ng/ml, 11 ng/mL, 12 ng/ml, 13 ng/mL, 14 ng/ml, 15 ng/ml, 16 ng/ml, 17 ng/ml, 18 ng/ml, 19 ng/ml, 20 ng/ml, 21 ng/mL, 22 ng/ml, 23 ng/ml, 24 ng/ml, 25 ng/ml, 26 ng/ml, 27 ng/ml, 28 ng/ml, 29 ng/ml, 30 ng/ml, 31 ng/ml, 32 ng/ml, 33 ng/ml, 34 ng/ml, 35 ng/ml, 36 ng/ml, 37 ng/ml, 38 ng/ml, 39 ng/ml, 40 ng/ml, 41 ng/ml, 42 ng/ml, 43 ng/ml, 44 ng/ml, 45 ng/ml, 46 ng/ml, 47 ng/ml, 48 ng/ml, 49 ng/ml, 50 ng/ml, 1-5 ng/ml, 1-10 ng/mL, 1-20 ng/ml, 1-30 ng/mL, 1-40 ng/ml, 1-50 ng/ml, 5-10 ng/ml, 5-20 ng/ml, 10-20 ng/ml, 10-30 ng/mL, 10-40 ng/ml, 10-50 ng/mL, 20-30 ng/ml, 20-40 ng/ml, 20-50 ng/ml, 30-40 ng/mL, 30-50 ng/ml, 40-50 ng/ml, 1-100 ng/ml, 50-100 ng/ml, 60-100 ng/ml, 70-100 ng/ml, 80-100 ng/ml, 90-100 ng/ml, 10-100 ng/ml, 50-200 ng/ml, 100-200 ng/ml, 50-300 ng/ml, 100-300 ng/ml, 200-300 ng/mL, 50-400 ng/ml, 100-400 ng/ml, 200-400 ng/ml, 300-400 ng/ml, 50-500 ng/ml, 100-500 ng/ml, 200-500 ng/ml, 300-500 ng/ml, 400 to 500 ng/ml, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 g/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5 -9 μg/mL, 0.5-10 μg/mL, and the like.

In some instances, the effective concentration of a particular differentiation factor in solution, e.g., cell culture media, may range from 1 nM to 100 μM or more, including but not limited to, e.g., 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 1-2 nM, 1-3 nM, 1-4 nM, 1-5 nM, 1-6 nM, 1-7 nM, 1-8 nM, 1-9 nM, 1-10 nM, 1.5 nM, 1.5-2 nM, 1.5-3 nM, 1.5-4 nM, 1.5-5 nM, 1.5-6 nM, 1.5-7 nM, 1.5-8 nM, 1.5-9 nM, 1.5-10 nM, 2-3 nM, 2-4 nM, 2-5 nM, 2-6 nM, 2-7 nM, 2-8 nM, 2-9 nM, 2-10 nM, 3-4 nM, 3-5 nM, 3-6 nM, 3-7 nM, 3-8 nM, 3-9 nM, 3-10 nM, 4-5 nM, 4-6 nM, 4-7 nM, 4-8 nM, 4-9 nM, 4-10 nM, 5-6 nM, 5-7 nM, 5-8 nM, 5-9 nM, 5-10 nM, 6-7 nM, 6-8 nM, 6-9 nM, 6-10 nM, 7-8 nM, 7-9 nM, 7-10 nM, 8-9 nM, 8-10 nM, 9-10 nM, 5-15 nM, 5-20 nM, 5-25 nM, 5-30 nM, 5-35 nM, 5-40 nM, 5-45 nM, 5-50 nM, 10-15 nM, 10-20 nM, 10-25 nM, 10-30 nM, 10-35 nM, 10-40 nM, 10-50 nM, 15-20 nM, 15-25 nM, 15-30 nM, 15-35 nM, 15-40 nM, 15-45 nM, 15-50 nM, 20-25 nM, 20-30 nM, 20-35 nM, 20-40 nM, 20-45 nM, 20-50 nM, 25-30 nM, 25-35 nM, 25-40 nM, 25-45 nM, 25-50 nM, 30-35 nM, 30-40 nM, 30-45 nM, 30-50 nM, 35-40 nM, 35-45 nM, 35-50 nM, 40-45 nM, 40-50 nM, 45-50 nM, 10-100 nM, 20-100 nM, 30-100 nM, 40-100 nM, 50-100 nM, 60-100 nM, 70-100 nM, 80-100 nM, 90-100 nM, 50-150 nM, 50-200 nM, 50-250 nM, 50-300 nM, 50-350 nM, 50-400 nM, 50-450 nM, 50-500 nM, 10-150 nM, 10-200 nM, 10-250 nM, 10-300 nM, 10-350 nM, 10-400 nM, 10-450 nM, 10-500 nM, 100-150 nM, 100-200 nM, 100-250 nM, 100-300 nM, 100-350 nM, 100-400 nM, 100-450 nM, 100-500 nM, 200-500 nM, 300-500 nM,400-500 nM, 100 nM, 150 nM, 200 nM, 250 nM, 300 nM, 350 nM, 400 nM, 450 nM,500 nM, 550 nM, 600 nM, 650 nM, 700 nM, 750 nM, 800 nM, 850 nM,900 nM, 950 nM, 200-400 nM, 300-500 nM, 400-600 nM, 500-700 nM, 600-800 nM, 700-900 nM, 800 nM to 1 μM, 0.5-1 μM, 0.5-1.5 μM, 0.5-2 μM, 0.5-2.5 μM, 0.5-3 μM, 0.5-3.5 μM, 0.5-4 μM, 0.5-4.5 μM, 0.5-5 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 M, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 1-2 μM, 1-3 μM, 1-4 μM, 1-5 μM, 1-6 μM, 1-7 μM, 1-8 μM, 1-9 μM, 1-10 μM, 1.5 μM, 1.5-2 μM, 1.5 -3 μM, 1.5-4 μM, 1.5-5 μM, 1.5-6 μM, 1.5-7 μM, 1.5-8 μM, 1.5-9 μM, 1.5-10 μM, 2-3 μM, 2-4 μM, 2-5 μM, 2 - 6 μM, 2-7 μM, 2-8 μM, 2-9 μM, 2-10 μM, 3-4 μM, 3-5 μM, 3-6 μM, 3-7 μM, 3-8 μM, 3-9 M, 3-10 μM, 4-5 μM, 4-6 μM, 4-7 μM, 4-8 μM, 4-9 μM, 4-10 μM, 5-6 μM, 5-7 μM, 5-8 μM, 5-9 μM, 5-10 μM, 6-7 μM, 6-8 μM, 6-9 μM, 6-10 μM, 7-8 μM, 7-9 μM, 7-10 μM, 8-9 μM, 8-10 μM, 9 - 10 μM, 5-15 μM, 5-20 μM, 5-25 μM, 5-30 μM, 5-35 μM, 5-40 μM, 5-45 μM, 5-50 μM, 10-15 μM, 10-20 μM, 10-25 μM, 10-30 μM, 10-35 μM, 10-40 μM, 10-50 μM, 15-20 μM, 15-25 μM, 15-30 μM, 15-35 μM, 15-40 μM, 15-45 M, 15-50 μM, 20-25 μM, 20-30 μM, 20-35 μM, 20-40 μM, 20-45 μM, 20-50 μM, 25-30 μM, 25-35 μM, 25-40 M, 25-45 μM, 25-50 μM, 30-35 μM, 30-40 μM, 30-45 M, 30-50 μM, 35-40 μM, 35-45 μM, 35-50 μM, 40-45 μM, 40-50 μM, 45-50 μM, 10-100 μM, 20-100 μM, 30-100 μM, 40-100 μM, 50-100 μM, 60-100 μM, 70-100 μM, 80-100 μM, 90-100 μM, and the like.

In some instances, the effective concentration of a particular differentiation factor will be below a critical concentration such that the induction produces the desired effect essentially without undesirable effects. As used herein, the term “critical concentration” refers to a concentration of induction agent above which undesirable effects are produced. Undesirable effects that may be the result of a concentration exceeding the critical concentration include but are not limited to, e.g., off-target effects (off-target activation of signaling, off-target inhibition of signaling), reduction or loss of function (e.g., loss of desired activator function, loss of desired inhibitor function) reduction of cell viability, increase in cell mortality, lineage restriction towards an undesired cell type, differentiation into an undesired cell type, loss of expression of a particular desired marker, etc. Whether a particular induction agent will have a critical concentration and what the critical concentrations of those agents having a critical concentration are will depend on the agent and the specific conditions in which the agent is used.

In some instances, cells of the instant disclosure may be contacted with multiple differentiation factors in order achieve a desired cell type of derivative thereof. In some instances, a particular composition will contain two or more agents such that a particular cell culture is simultaneously contacted with multiple factors. In some instances, a particular series of factors may be used, one at a time, in generating a desired cell type such that a particular cell culture is successively contacted with multiple agents.

The duration of contact of a particular factor composition with a particular cell type, in some instances, may be referred to as the “exposure time” and exposure times may range from a day to weeks or more, including but not limited to e.g., 1 day, 1.5 days, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, 10 days, 11 days, 12, days, 13, days, 14 days, 15, days, etc. As used herein, exposure times are, in some instances, referred as consisting essentially of, e.g., 24 hours, indicating that the exposure time may be longer or shorter than that specified including those exposure times that are longer or shorter but do not materially affect the basic outcome of the particular exposure. As such, in some instances where a particular exposure is more time sensitive such that under or over exposure, e.g., of more or less than 1 hour, materially affects the outcome of the exposure, a time period consisting essentially of, e.g., 24 hours, will be interpreted to refer to a time period ranging from about 23 hours to about 25 hours. In some other instances where a particular exposure is less time sensitive such that under or over exposure, e.g., of more than 12 hours, does not materially affect the outcome of the exposure, a time period consisting essentially of, e.g., 24 hours will mean a time period ranging from about 12 hours or less to about 36 hours or more. In some instances, depending on the context, an exposure period consisting essentially of 24 hours may refer to an exposure time of 22-26 hours, 21-27 hours, 20-28 hours, 19-29 hours, 18-30 hours, etc.

In some instances, time periods of exposure may be pre-determined such that cells are contacted with factors according to a schedule set forth prior to the contacting. In some instances, the time period of exposure, whether pre-determined or otherwise, may be modulated according to some feature or characteristic of the cells and/or cell culture, including but not limited to, e.g., cell morphology, cell viability, cell appearance, cellular behaviors, cell number, culture confluence, marker expression, etc.

### Screening Assays

Methods are provided for determining the activity of a candidate agent on a vascularized organoid, the method comprising contacting the candidate agent with one or a panel of VOs and determining the effect of the agent on morphologic, genetic, or functional parameters.

In screening assays for the small molecules, the effect of adding a candidate agent to cells in culture is tested with a panel of cells and cellular environments, where the cellular environment includes one or more of: electrical stimulation including alterations in ionicity, drug stimulation, and the like, and where panels of cells may vary in genotype, in prior exposure to an environment of interest, in the dose of agent that is provided, etc., where usually at least one control is included, for example a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional, and genetic changes.

In the screening assays for genetic agents, polynucleotides are added to one or more of the cells in a panel in order to alter the genetic composition of the cell. The output parameters are monitored to determine whether there is a change in phenotype. In this way, genetic sequences are identified that encode or affect expression of proteins in pathways of interest. The results can be entered into a data processor to provide a screening results dataset. Algorithms are used for the comparison and analysis of screening results obtained under different conditions.

Parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases, the molecular entities comprising the epitope are from two or more substances and comprise a defined structure; examples include combinatorically determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs, or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, New York, (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural, or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars, or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs. The-anomer of deoxyribose may be used, where the base is inverted with respect to the natural-anomer. The 2′-OH of the ribose sugar may be altered to form 2′-O-methyl or 2′-O-allyl sugars, which provides resistance to degradation without comprising affinity.

Agents are screened for biological activity by adding the agent to at least one and usually a plurality of cells, in one or in a plurality of environmental conditions, e.g. following stimulation with a β-adrenergic agonist, following electric or mechanical stimulation, etc. The change in parameter readout in response to the agent is measured, desirably normalized, and the resulting screening results may then be evaluated by comparison to reference screening results, e.g. with cells having other mutations of interest, normal cardiac fibroblasts, cardiac fibroblasts derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluorescence, e.g., by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody-or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, NY, 2000; Freeman et al. (1999) Biotechniques 26(1):112-225; Kawamoto et al. (1999) Genome Res 9(12):1305-12; and Chen et al. (1998) Genomics 51(3):313-24, for examples.

The comparison of a screening results obtained from a test compound, and a reference screening results(s) is accomplished by the use of suitable deduction protocols, Al systems, statistical comparisons, etc. Preferably, the screening results is compared with a database of reference screening results. A database of reference screening results can be compiled. These databases may include reference results from panels that include known agents or combinations of agents, as well as references from the analysis of cells treated under environmental conditions in which single or multiple environmental conditions or parameters are removed or specifically altered. Reference results may also be generated from panels containing cells with genetic constructs that selectively target or modulate specific cellular pathways.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

In some instances, organoids generated according to the methods as described herein are introduced into a host animal and the host animal may be administered a pharmacological agent in order to screen for a response from the introduced cells. In some instances, the cells of the in vivo assay may be directly evaluated, e.g., for an intrinsic response to a pharmacological agent. In some instances, the host animal of the in vivo assay may be evaluated as an indirect measurement of the response of the cells to the pharmacological agent.

For convenience, the systems of the subject invention may be provided in kits. The kits could include the cells to be used, which may be frozen, refrigerated or treated in some other manner to maintain viability, reagents for measuring the parameters, and software for preparing the screening results. The software will receive the results and perform analysis and can include reference data. The software can also normalize the results with the results from a control culture. The composition may optionally be packaged in a suitable container with written instructions for a desired purpose, such as screening methods, and the like.

### Factors

In some instances, a factor useful in a particular induction composition may include an activator or inhibitor of the TGF-beta (transforming growth factor β (TGF-β)) pathway. Activators and inhibitors of the TGF-beta pathway include small molecule activators, small molecule inhibitors, peptide activators, peptide inhibitors, antibodies, nucleic acid activators, nucleic acid inhibitors, and the like that activate or inhibit at least one component of the TGF-beta pathway resulting in a corresponding activation or inhibition in cellular TGF-beta signaling. Components and downstream effectors of the TGF-beta pathway include but are not limited to, e.g., 14-3-3 e (UniProtID P62258), ark (UniProtID Q6ZNA4), axin1 (UniProtID 015169), bambi (UniProtID Q13145), beta arrestin 2 (UniProtID P32121), beta catenin (UniProtID P35222), beta glycan (UniProtID Q03167), camkiia (UniProtID Q9UQM7), caveolin-1 (UniProtID Q03135), ctgf (UniProtID P29279), dab2 (UniProtID P98082), dapper2 (UniProtID Q5SW24), daxx (UniProtID Q9UER7), eif2a (UniProtID Q9BY44), elf (UniProtID Q01082), endofin (UniProtID Q7Z3T8), fkbp12 (UniProtID P62942), gadd34 (UniProtID O75807), grb2 (UniProtID P62993), itch (UniProtID Q96J02), km23-1 (UniProtID Q9NP97), nedd4-2 (UniProtID Q96PU5), ocln (UniProtID Q16625), p70s6k (UniProtID P23443), par6 (UniProtID Q9NPB6), pdk1 (UniProtID O15530), pml (UniProtID P29590), ppp1ca (UniProtID P62136), ppp2ca (UniProtID P67775), ppp2cb (UniProtID P62714), ppp2r2a (UniProtID P63151), rhoa (UniProtID P61586), sara (UniProtID O95405), shc (UniProtID P29353), smad2 (UniProtID Q15796), smad3 (UniProtID P84022), smad4 (UniProtID Q13485), smad7 (UniProtID 015105), smurf1 (UniProtID Q9HCE7), smurf2 (UniProtID Q9HAU4), snon (UniProtID P12757), sos1 (UniProtID Q07889), strap (UniProtID Q9Y3F4), tab1 (UniProtID Q15750), tab2 (UniProtID Q9NYJ8), tak1 (UniProtID 043318), TGFB1 (UniProtID P01137), TGFB2 (UniProtID P61812), TGFB3 (UniProtID P10600), tgfbr1 (UniProtID P36897), tgfbr2 (UniProtID P37173), trap-1 (UniProtID O60466), wwp1 (UniProtID Q9H0M0), xiap (UniProtID P98170), yap65 (UniProtID P46937), and the like.

Activators of the TGF-beta pathway include but are not limited to, e.g., TGF-beta family ligands (e.g., TGF-beta proteins and other activators of TGF-beta receptors) and portions thereof, Activin A, TGF-beta1, TGF-beta2, TGF-beta3 IDE1/2 (IDE1 (1-[2-[(2-Carboxyphenyl)methylene]hydrazide]heptanoic acid), IDE2 (Heptanedioic acid-1-(2-cyclopentylidenehydrazide)), Nodal, and the like. In some instances, activation of the TGF-beta pathway may be achieved through repression of the TGF-beta pathway inhibitor, e.g., including but not limited to the use of an inhibitory nucleic acid targeting an inhibitor of the TGF-beta pathway or an antibody or small molecule directed to a TGF-beta pathway inhibitor.

Inhibitors of the TGF-beta pathway, e.g. inhibitors of the SMAD pathway, include but are not limited to, e.g., A-83-01 (3-(6-Methyl-2-pyridinyl)-N-phenyl-4-(4-quinolinyl)-1 H-pyrazole-1-carbothioamide), D4476 (4-[4-(2,3-Dihydro-1,4-benzodioxin-6-yl)-5-(2-pyridinyl)-1 H-imidazol-2-yl]benzamide), GW 788388 (4-[4-[3-(2-Pyridinyl)-1 H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids, and the like.

In some instances, an inducing agent useful in a particular induction composition may include an activator or inhibitor of the Wnt pathway. Activators and inhibitors of the Wnt pathway include small molecule activators, small molecule inhibitors, peptide activators, peptide inhibitors, antibodies, nucleic acid activators, nucleic acid inhibitors, and the like that activate or inhibit at least one component of the Wnt pathway resulting in a corresponding activation or inhibition in cellular Wnt signaling. Components and downstream effectors of the Wnt pathway include but are not limited to, e.g., cthrc1 (UniProtID Q96CG8), dkk1 (UniProtID 094907), fzd1 (UniProtID Q9UP38), fzd10 (UniProtID Q9ULW2), fzd2 (UniProtID Q14332), fzd4 (UniProtID Q9ULV1), fzd5 (UniProtID Q13467), fzd6 (UniProtID 060353), fzd7 (UniProtID 075084), fzd8 (UniProtID Q9H461), fzd9 (UniProtID 000144), igfbp4 (UniProtID P22692), kremen 1 (UniProtID Q96MU8), kremen 2 (UniProtID Q8NCW0), Irp5 (UniProtID 075197), Irp6 (UniProtID O75581), prr (UniProtID 075787), ror2 (UniProtID Q01974), rspo1 (UniProtID Q2MKA7), ryk (UniProtID P34925), wnt inhibitory 1 (UniProtID Q9Y5W5), wnt1 (UniProtID P04628), wnt2 (UniProtID P09544), wnt3 (UniProtID P56703), wnt3a (UniProtID P56704), wnt5a (UniProtID P41221), wnt7a (UniProtID 000755), wnt7b (UniProtID P56706), CTNNB1 (UniProtID P35222), GSK3A (UniProtID P49840), GSK3B (UniProtID P49841), TNKS1 (UniProtID O95271), TNKS2 (UniProtID Q9H2K2) and the like.

Activators of the WNT pathway include but are not limited to, e.g., CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), WNT family ligands (e.g., including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, etc.), RSPO co-agonists (e.g., RSPO2), lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo [4,5-j]phenanthridine), CID 11210285 hydrochloride (2-Amino-4-(3,4-(methylenedioxy) benzylamino)-6-(3-methoxyphenyl)pyrimidine hydrochloride), WAY-316606, (hetero) arylpyrimidines, IQ1, QS11, SB-216763, DCA, and the like. In some instances, activation of the Wnt pathway may be achieved through repression of a Wnt pathway inhibitor, e.g., including but not limited to the use of an inhibitory nucleic acid targeting an inhibitor of the Wnt pathway or an antibody or small molecule directed to a Wnt pathway inhibitor.

Inhibitors of the WNT pathway include but are not limited to, e.g., C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Ant1.4Br, Ant 1.4Cl, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1,1-Dimethylethoxy)carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1(6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, Wnt inhibitory nucleic acids, and the like.

In some instances, a Wnt activator or inhibitor useful in the methods described herein may include those described in, e.g., Dodge and Lum et al. Annu Rev Pharmacol Toxicol. 2011; 51:289-310; Chen et al. Am J Physiol Gastrointest Liver Physiol. 2010 August; 299(2):G293-300; Baker and Clevers, Nat Rev Drug Discov. 2006 December; 5(12):997-1014; Meijer et al. Trends Pharmacol Sci. 2004 September; 25(9):471-80; and Lepourcelet et al. Cancer Cell. 2004 January; 5(1):91-102, the disclosures of which are incorporated herein by reference in their entirety.

In some instances, an inducing agent useful in a particular induction composition may include an activator or inhibitor of the FGF pathway. In some instances, an activator or inhibitor of the FGF pathway may also include activators or inhibitors of related signal transduction pathways including but not limited to, e.g., the MAPK/ERK signal transduction pathway. Activators and inhibitors of the FGF pathway include small molecule activators, small molecule inhibitors, peptide activators, peptide inhibitors, antibodies, nucleic acid activators, nucleic acid inhibitors, and the like that activate or inhibit at least one component of the FGF pathway resulting in a corresponding activation or inhibition in cellular FGF signaling. Components and downstream effectors of the FGF pathway include but are not limited to, e.g., akt1 (UniProtID P31749), beta-klotho (UniProtID Q86Z14), camkiia (UniProtID Q9UQM7), cbl (UniProtID P22681), cortactin (UniProtID Q14247), e-cadherin (UniProtID P12830), erk1 (UniProtID P27361), erk2 (UniProtID P28482), FGF1 (UniProtID P05230), FGF16 (UniProtID O60258), FGF17 (UniProtID O60258), FGF18 (UniProtID O76093), FGF19 (UniProtID 095750), FGF2 (UniProtID P09038), fgf23 (UniProtID Q9GZV9), FGF4 (UniProtID P08620), FGF6 (UniProtID P10767), FGF8 (UniProtID P55075), FGF9 (UniProtID P31371), fgfr1 (UniProtID P11362), fgfr2 (UniProtID P21802), fgfr2b (UniProtID P21802-18), FGFR2c (UniProtID P21802-5), FGFR3c (UniProtID P22607-1), FGFR4 (UniProtID P22455), fos (UniProtID P01100), frs2 (UniProtID Q8WU20), gab1 (UniProtID Q13480), grb2 (UniProtID P62993), hgf (UniProtID P14210), jun (UniProtID P05412), klotho (UniProtID Q9UEF7), mapk 14 (UniProtID Q16539), met (UniProtID P08581), mkp-3 (UniProtID Q16828), mmp9 (UniProtID P14780), n-cad-ctf1 (UniProtID P19022), n-cad-ctf2 (UniProtID P19022), n-cadherin (UniProtID P19022), ncam (UniProtID P13591), osteocalcin (UniProtID P02818), osteopontin (UniProtID P10451), p110-alpha (UniProtID P42336), p120ctn (UniProtID O60716), p90-rsk 1 (UniProtID Q15418), pak4 (UniProtID Q8WYL5), pak4 (UniProtID 096013), pdk1 (UniProtID O15530), pik3r1 (UniProtID P27986), plcgamma1 (UniProtID P19174), pro-e-cadherin (UniProtID P12830), pro-mmp9 (UniProtID P14780), ps1 (UniProtID gamma), pyk2 (UniProtID Q14289), runx2 (UniProtID Q13950), se-cad (UniProtID P12830), secad-ntf2 (UniProtID P12830), sef (UniProtID Q8NFM7), shc (UniProtID P29353), shp2 (UniProtID Q06124), sn-cad (UniProtID P19022), sos1 (UniProtID Q07889), sprouty2 (UniProtID O43597), src (UniProtID P12931), stat1 (UniProtID P42224), stat3 (UniProtID P40763), stat5b (UniProtID P51692), syndecan-2 (UniProtID P34741), syndecan-4 (UniProtID P31431), upa (UniProtID P00749), upar (UniProtID Q03405), and the like. Activators and inhibitors of the MAPK/ERK pathway include small molecule activators, small molecule inhibitors, peptide activators, peptide inhibitors, antibodies, nucleic acid activators, nucleic acid inhibitors, and the like that activate or inhibit at least one component of the MAPK/ERK pathway resulting in a corresponding activation or inhibition in cellular MAPK/ERK signaling. Components and downstream effectors of the MAPK/ERK pathway MAPK/ERK signaling include but are not limited to, e.g., a-raf (EntrezGeneID 369), ask1 (EntrezGeneID 4217), atf2 (EntrezGeneID 1386), cebpa (EntrezGeneID 1050), c-myc (EntrezGeneID 4609), creb (EntrezGeneID 1385), elk1 (EntrezGeneID 2002), erk5 (EntrezGeneID 5598), fos (EntrezGeneID 2353), grb2 (EntrezGeneID 2885), hexokinase type iv glucokinase (EntrezGeneID 2645), ikk-alpha (EntrezGeneID 1147), ikk-beta (EntrezGeneID 3551), jnk (EntrezGeneID 5599), jun (EntrezGeneID 3725), map2k1 (EntrezGeneID 5604), map2k2 (EntrezGeneID 5605), map2k4 (EntrezGeneID 6416), map2k5 (EntrezGeneID 5607), map2k6 (EntrezGeneID 5608), map2k7 (EntrezGeneID 5609), map3k1 (EntrezGeneID 4214), map3k11 (EntrezGeneID 4296), map3k12 (EntrezGeneID 7786), map3k13 (EntrezGeneID 9175), map3k14 (EntrezGeneID 9020), map3k2 (EntrezGeneID 10746), map3k3 (EntrezGeneID 4215), map3k4 (EntrezGeneID 4216), map3k7 (EntrezGeneID 6885), map3k8 (EntrezGeneID 1326), map4k1 (EntrezGeneID 11184), map4k3 (EntrezGeneID 8491), map4k5 (EntrezGeneID 11183), mapk1 (EntrezGeneID 5594), mapk10 (EntrezGeneID 5602), mapk11 (EntrezGeneID 5600), mapk12 (EntrezGeneID 6300), mapk13 (EntrezGeneID 5603), mapk14 (EntrezGeneID 1432), mapk3 (EntrezGeneID 5595), mapk9 (EntrezGeneID 5601), max (EntrezGeneID 4149), mef2 polypeptide a (EntrezGeneID 4205), mef2 polypeptide c (EntrezGeneID 4208), mef2b (EntrezGeneID 4207), mef2 polypeptide d (EntrezGeneID 4209), mek3 (EntrezGeneID 5606), mknk2 (EntrezGeneID 2872), mnk1 (EntrezGeneID 8569), msk1 (EntrezGeneID 9252), ngf r (EntrezGeneID 4804), ngfb (EntrezGeneID 4803), nik (EntrezGeneID 9448), pak1 (EntrezGeneID 5058), pak2 (EntrezGeneID 5062), pp2a (EntrezGeneID 5528), ptprr (EntrezGeneID 5801), rac1 (EntrezGeneID 5879), raf1 (EntrezGeneID 5894), ras (EntrezGeneID 3265), rps6ka1 (EntrezGeneID 6195), shc (EntrezGeneID 6464), sos1 (EntrezGeneID 6654), sp1 (EntrezGeneID 6667), src (EntrezGeneID 6714), stat1 (EntrezGeneID 6772), stat3 (EntrezGeneID 6774), tert (EntrezGeneID 7015), and the like.

Activators of the FGF pathway and/or the MAPK/ERK pathway include but are not limited to, e.g., FGF family ligands (e.g., FGF1, FGF2, FGF-3, FGF-4, FGF-5, FGF-6, KGF/FGF-7, FGF-8, FGF-9, FGF-10, FGF-11, FGF-12, FGF-13, FGF-15, FGF-16, FGF-17, FGF-19, FGF-20, FGF-21, FGF-22, FGF-23, etc.), SUN 11602 (4-[[4-[[2-[(4-Amino-2,3,5,6-tetramethylphenyl)amino]acetyl]methylamino]-1-piperidinyl]methyl]benzamide), t-Butylhydroquinone, U-46619, C2 Ceramide, Lactosyl Ceramide, Angiotensin II, Baicalin, and the like. In some instances, activation of the FGF pathway and/or the MAPK/ERK pathway may be achieved through repression of the a FGF pathway and/or the MAPK/ERK pathway inhibitor, e.g., including but not limited to the use of an inhibitory nucleic acid targeting an inhibitor of the FGF pathway and/or the MAPK/ERK pathway or an antibody or small molecule directed to a FGF pathway inhibitor and/or MAPK/ERK pathway inhibitor.

Inhibitors of the FGF pathway and/or the MAPK/ERK pathway and or the p38/JNK/MAPK cascade include but are not limited to, e.g., AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide), PD173074 (N-[2-[[4-(Diethylamino)butyl]amino]-6-(3,5-dimethoxyphenyl)pyrido[2,3-d]pyrimidin-7-yl]-N′-(1, 1-dimethylethyl)urea), FIIN 1 hydrochloride (N-(3-((3-(2,6-dichloro-3,5-dimethoxyphenyl)-7-(4-(diethylamino)butylamino)-2-oxo-3,4-dihydropyrimido[4,5-d]pyrimidin-1(2H)-yl)methyl)phenyl)acrylamide), PD 161570 (N-[6-(2,6-Dichlorophenyl)-2-[[4-(diethylamino)butyl]amino]pyrido[2,3-d]pyrimidin-7-yl]-N′-(1,1-dimethylethyl)urea), SU 5402 (2-[(1,2-Dihydro-2-oxo-3H-indol-3-ylidene)methyl]-4-methyl-1H-pyrrole-3-propanoic acid), SU 6668 (5-[1,2-Dihydro-2-oxo-3H-indol-3-ylidene)methyl]-2,4-dimethyl-1H-pyrrole-3-propanoic acid), PD0325901 (N-[(2R)-2,3-Dihydroxypropoxy]-3,4-difluoro-2-[(2-fluoro-4-iodophenyl) amino]-benzamide), BIX 02189 ((3Z)-3-[[[3-[(Dimethylamino)methyl]phenyl]amino]phenylmethylene]-2,3-dihydro-N,N-dimethyl-2-oxo-1H-indole-6-carboxamide), FR 180204 (5-(2-Phenyl-pyrazolo[1,5-a]pyridin-3-yl)-1H-pyrazolo[3,4-c]pyridazin-3-ylamine), Pluripotin (N-[3-[7-[(1,3-Dimethyl-1H-pyrazol-5-yl)amino]-1,4-dihydro-1-methyl-2-oxopyrimido [4,5-d]pyrimidin-3(2H)-yl]-4-methylphenyl]-3-(trifluoromethyl) benzamide), TCS ERK 11e (4-[2-[(2-Chloro-4-fluorophenyl)amino]-5-methyl-4-pyrimidinyl]-N-[(1S)-1-(3-chlorophenyl)-2-hydroxyethyl]-1H-pyrrole-2-carboxamide), TMCB (2-(4,5,6,7-Tetrabromo-2-(dimethylamino)-1H-benzo[d]imidazol-1-yl)acetic acid), XMD 8-92 (2-[[2-Ethoxy-4-(4-hydroxy-1-piperidinyl)phenyl]amino]-5,11-dihydro-5,11-dimethyl-6H-pyrimido [4,5-b][1,4]benzodiazepin-6-one), SU5402, AZD4547, BGJ398, AL 8697, AMG 548, CMPD-1, DBM 1285 dihydrochloride, EO 1428, JX 401, ML 3403, RWJ 67657, SB 202190, SB-203580, SB 239063, SB 706504, Scio-469, SKF 86002 dihydrochloride, SX 011, TA 01 (4-(2-(2,6-Difluorophenyl)-4-(fluorophenyl)-1H-imidazol-5-yl)pyridine), TA 02 (4-(2-(2-Fluorophenyl)-4-(fluorophenyl)-1H-imidazol-5-yl)pyridine), TAK 715, VX-702, VX-745, antibodies to FGF and/or MAPK pathway components including ligands and receptors, FGF and/or MAPK inhibitory nucleic acids, and the like.

In some instances, a FGF or MAPK activator or inhibitor useful in the methods described herein may include those described in, e.g., English and Cobb, Trends Pharmacol Sci. 2002 January; 23(1):40-5, the disclosure of which is incorporated herein by reference in its entirety.

In some instances, an agent useful in a particular composition may include an activator or inhibitor of the BMP pathway. Activators and inhibitors of the BMP pathway include small molecule activators, small molecule inhibitors, peptide activators, peptide inhibitors, antibodies, nucleic acid activators, nucleic acid inhibitors, and the like that activate or inhibit at least one component of the BMP pathway resulting in a corresponding activation or inhibition in cellular BMP signaling. Components and downstream effectors of the BMP pathway include but are not limited to, e.g., bambi (UniProtID Q13145), bmp2 (UniProtID P12643), bmp4 (UniProtID P12644), bmp6 (UniProtID P22004), bmp7 (UniProtID P18075), bmpr1a (UniProtID P36894), bmpr1b (UniProtID O00238), bmpr2 (UniProtID Q13873), cer1 (UniProtID 095813), chrd (UniProtID Q9H2X0), chrdl1 (UniProtID Q9BU40), endofin (UniProtID Q7Z3T8), erk2 (UniProtID P28482), fetua (UniProtID P02765), fs (UniProtID P19883), gadd34 (UniProtID O75807), grem1 (UniProtID O60565), gsk3beta (UniProtID P49841), nog (UniProtID Q13253), nup214 (UniProtID P35658), ppm1a (UniProtID P35813), ppp1ca (UniProtID P62136), rgma (UniProtID Q96B86), rgmb (UniProtID Q6NW40), rgmc (UniProtID Q6ZVN8), scp1 (UniProtID Q9GZU7), scp2 (UniProtID O14595), scp3 (UniProtID O15194), ski (UniProtID P12755), smad1 (UniProtID Q15797), smad4 (UniProtID Q13485), smad5 (UniProtID Q99717), smad6 (UniProtID O43541), smad7 (UniProtID O15105), smad8a (UniProtID O15198), smurf1 (UniProtID Q9HCE7), smurf2 (UniProtID Q9HAU4), tab1 (UniProtID Q15750), tab2 (UniProtID Q9NYJ8), tak1 (UniProtID 043318), usag1 (UniProtID Q6X4U4), xiap (UniProtID P98170),, and the like.

Activators of the BMP pathway include but are not limited to, e.g., BMP family ligands (e.g., BMP2, BMP4, BMP7, etc.), Alantolactone, FK506, isoliquiritigenin, 4′-hydroxychalcone, and the like. In some instances, activation of the BMP pathway may be achieved through repression of a BMP pathway inhibitor, e.g., including but not limited to the use of an inhibitory nucleic acid targeting an inhibitor of the BMP pathway or an antibody or small molecule directed to a BMP pathway inhibitor.

Inhibitors of the BMP pathway include but are not limited to, e.g., NOGGIN, CHORDIN, LDN-193189 (4-[6-[4-(1-Piperazinyl)phenyl]pyrazolo[1,5-a]pyrimidin-3-yl]-quinoline hydrochloride), DMH1 (4-[6-[4-(1-Methylethoxy)phenyl]pyrazolo[1,5-a]pyrimidin-3-yl]-quinoline), Dorsomorphin (6-[4-[2-(1-Piperidinyl)ethoxy]phenyl]-3-(4-pyridinyl)-pyrazolo[1,5-a]pyrimidine dihydrochloride), K 02288 (3-[(6-Amino-5-(3,4,5-trimethoxyphenyl)-3-pyridinyl]phenol), ML 347 (5-[6-(4-Methoxyphenyl)pyrazolo[1,5-a]pyrimidin-3-yl]quinoline), DMH-1, antibodies to BMPs and BMP receptors, BMP inhibitory nucleic acids, and the like.

In some instances, an agent useful in a particular composition may include an activator or inhibitor of the retinoic acid signaling pathway. Activators and inhibitors of the retinoic acid signaling pathway include small molecule activators, small molecule inhibitors, peptide activators, peptide inhibitors, antibodies, nucleic acid activators, nucleic acid inhibitors, and the like that activate or inhibit at least one component of the retinoic acid signaling pathway resulting in a corresponding activation or inhibition in cellular retinoic acid signaling. Components and downstream effectors of the retinoic acid signaling pathway include but are not limited to, e.g., CRABP (e.g., Accession: NP_004369), TRAIL (e.g., Accession: NP_003801), TRAILR1 (e.g., Accession: NP_003835), TRAILR2 (e.g., Accession: NP_003833), DAP3 (e.g., Accession: NP_001186780), FADD (e.g., Accession: CAG33019), FLIP (e.g., Accession: NP_001294972), Caspase 8 (e.g., Accession: AAD24962), BID (e.g., Accession: NP_001304162), tBID (e.g., Accession: P55957), APAF1 (e.g., Accession: ABQ59028), Caspase 9 (e.g., Accession: P55211), PARPs (e.g., Accession: AAH14206), RAR (e.g., Accession: NP_001138773 and components thereof e.g., AF2 domain, AF1 domain, DBD domain, and the like. Activators and inhibitors of the retinoic acid signaling include but are not limited to e.g., Tretinoin, Retinol palmitate, Etretinate, Isotretinoin, Adapalene, Tazarotene, Tamibarotene, Retinol acetate, Acitretin, Alitretinoin, Bexarotene, Isotretinoin anisatil, Motretinide, Vitamin A, Retinol propionate, and the like. In some instances, useful modulators of the retinoic acid signaling pathway include retinoid agonist, including but not limited to e.g., all-trans retinoic acid, TTNPB, AM580 and the like.

In some instances, factor useful in a particular composition may include an activator or inhibitor of the Hedgehog pathway. Activators and inhibitors of the Hedgehog pathway include small molecule activators, small molecule inhibitors, peptide activators, peptide inhibitors, antibodies, nucleic acid activators, nucleic acid inhibitors, and the like that activate or inhibit at least one component of the Hedgehog pathway resulting in a corresponding activation or inhibition in cellular Hedgehog signaling. Components and downstream effectors of the Hedgehog pathway include but are not limited to, e.g., akt1 (UniProtID P31749), beta arrestin2 (UniProtID P32121), boc (UniProtID Q9BWV1), cdo (UniProtID Q4KMG0), dhh (UniProtID 043323), gas1 (UniProtID P54826), gli2 (UniProtID P10070), grk2 (UniProtID P25098), hhat (UniProtID Q5VTY9), hhip (UniProtID Q96QV1), ihh (UniProtID Q14623), Irpap1 (UniProtID P30533), megalin (UniProtID P98164), p110-alpha (UniProtID P42336), pik3r1 (UniProtID P27986), ptch1 (UniProtID Q13635), ptch2 (UniProtID Q9Y6C5), pthrp (UniProtID P12272), shh (UniProtID Q15465), sil (UniProtID Q15468), smo (UniProtID Q99835), tgf-beta2 (UniProtID P61812), and the like.

Activators of the Hedgehog pathway include but are not limited to, e.g., Hedgehog family ligands (Hh, Shh, Ihh, Dhh, etc.) and fragments thereof, benzothiophene smoothened agonists, SAG (Hh-Ag1.3), SAG21k (3-chloro-4,7-difluoro-N-(4-methoxy-3-(pyridin-4-yl)benzyl)-N-((1r,4r)-4-(methylamino)cyclohexyl)benzo[b]thiophene-2-carboxamide), Hh-Ag1.1, Hh-Ag1.5, purmorphamine, and the like. In some instances, activation of the Hedgehog pathway may be achieved through repression of a Hedgehog pathway inhibitor, e.g., including but not limited to the use of an inhibitory nucleic acid targeting an inhibitor of the Hedgehog pathway or an antibody or small molecule directed to a Hedgehog pathway inhibitor.

Inhibitors of the Hedgehog pathway include but are not limited to, e.g., Hedgehog antagonists that target smoothened (SMO), Hedgehog antagonists that target patched (PTCH), Hedgehog antagonists that target Gli, cyclopamine and analogs and derivatives thereof, cyclopamine-competitive antagonists, IPI-926 (Saridegib), LDE225 (sonidegib), itraconazole, GDC-0449 (vismodegib), SANT1, KAAD-cyclopamine, LEQ506, PF-04449913, TAK-441, BMS833923 (XL-139), LY2940680, and inhibitory nucleic acids targeting SMO, inhibitory nucleic acids targeting a Hedgehog, inhibitory nucleic acids targeting PTCH, inhibitory nucleic acids targeting Gli (e.g., siRNA targeting Gli1), arsenic trioxide, and the like.

In some instances, Hedgehog pathway activators and Hedgehog pathway inhibitors include those agents described in, e.g., Chen et al. (2002) PNAS. 99(22):14071-14076; Frank-Kamenetsky, et al. (2002) J Biol. 1(2):10; Paladini et al. (2005) J Invest Dermatol. 125(4):638-46; Nakamura et al. (2014) J Cell. Physiol. ePub, Yun et al., Arch Pharm Res. 2012 August; 35(8):1317-33; the disclosures of which are incorporated herein by reference in their entirety.

In some instances, a factor useful in a particular induction composition may include an activator or inhibitor of the PI3K pathway. Activators and inhibitors of the PI3K pathway include small molecule activators, small molecule inhibitors, peptide activators, peptide inhibitors, antibodies, nucleic acid activators, nucleic acid inhibitors, and the like that activate or inhibit at least one component of the PI3K pathway resulting in a corresponding activation or inhibition in cellular PI3K signaling. Components and downstream effectors of the PI3K pathway include but are not limited to, e.g., arap3 (UniProtID Q8WWN8), arf1 (UniProtID P84077), arf5 (UniProtID P84085), arf6 (UniProtID P62330), arno (UniProtID Q99418), bam32 (UniProtID Q9UN19), blk (UniProtID P51451), bink (UniProtID Q8WV28), btk (UniProtID Q06187), centa1 (UniProtID O75689), cytohesin-1 (UniProtID Q15438), fgr (UniProtID P09769), foxo3a (UniProtID O43524), fyn (UniProtID P06241), grp1 (UniProtID O43739), hck (UniProtID P08631), h-ras isoform 1 (UniProtID P01112), h-ras isoform 2 (UniProtID P01112), hsp90 (UniProtID P07900), itk (UniProtID Q08881), k-ras isoform 2a (UniProtID P01116-1), k-ras isoform 2b (UniProtID P01116-2), lat (UniProtID O43561-2), Ick (UniProtID P06239), lyn (UniProtID P07948), n-ras (UniProtID P01111), p101 (UniProtID Q8WYR1), p110-alpha (UniProtID P42336), p110-beta (UniProtID P42338), p110D (UniProtID O00329), p55-gamma (UniProtID Q92569), p84 (UniProtID Q5UE93), p85-beta (UniProtID 000459), pdk1 (UniProtID O15530), PI3Kgamma (UniProtID P48736), PIK3R1 (UniProtID P27986), plcgamma1 (UniProtID P19174), plcgamma2 (UniProtID P16885), pten (UniProtID P60484), rac1 (UniProtID P63000), rap1a (UniProtID P62834), rhoa (UniProtID P61586), sgk1 (UniProtID 000141), ship (UniProtID 000145), ship2 (UniProtID O15357), src (UniProtID P12931), syk (UniProtID P43405), tapp1 (UniProtID Q9HB19), tapp2 (UniProtID Q9HB21), yes (UniProtID P07947), zap-70 (UniProtID P43403),, and the like.

Activators of the PI3K pathway include but are not limited to, e.g., PI3K family ligands, 740Y-P, Insulin receptor substrate (Tyr608) peptide (KKHTDDGYMPMSPGVA, SEQ ID NO:1), and the like. In some instances, an FGF signaling protein may serve as an activator of the PI3K pathway. In some instances, activation of the PI3K pathway may be achieved through repression of a PI3K pathway inhibitor, e.g., including but not limited to the use of an inhibitory nucleic acid targeting an inhibitor of the PI3K pathway or an antibody or small molecule directed to a PI3K pathway inhibitor.

Inhibitors of the PI3K pathway include but are not limited to, e.g., AS 252424 (5-[[5-(4-Fluoro-2-hydroxyphenyl)-2-furanyl]methylene]-2,4-thiazolidinedione), AS 605240 (5-(6-Quinoxalinylmethylene)-2,4-thiazolidine-2,4-dione), AZD 6482 ((-)-2-[[(1R)-1-[7-Methyl-2-(4-morpholinyl)-4-oxo-4H-pyrido[1,2-a]pyrimidin-9-yl]ethyl]amino]benzoic acid), BAG 956 (α,α,-Dimethyl-4-[2-methyl-8-[2-(3-pyridinyl)ethynyl]-1 H-imidazo[4,5-c]quinolin-1-yl]-benzeneacetonitrile), CZC 24832 (5-(2-Amino-8-fluoro[1,2,4]triazolo[1,5-a]pyridin-6-yl)-N-(1,1-dimethylethyl)-3-pyridinesulfonamide), GSK 1059615 (5-[[4-(4-Pyridinyl)-6-quinolinyl]methylene]-2,4-thiazolidenedione), KU 0060648 (4-Ethyl-N-[4-[2-(4-morpholinyl)-4-oxo-4H-1-benzopyran-8-yl]-1-dibenzothienyl]-1-piperazineacetamide), LY 294002 hydrochloride (2-(4-Morpholinyl)-8-phenyl-4H-1-benzopyran-4-one hydrochloride), 3-Methyladenine (3-Methyl-3H-purin-6-amine), PF 04691502 (2-Amino-8-[trans-4-(2-hydroxyethoxy)cyclohexyl]-6-(6-methoxy-3-pyridinyl)-4-methyl-pyrido[2,3-d]pyrimidin-7 (8H)-one), PF 05212384 (N-[4-[[4-(Dimethylamino)-1-piperidinyl]carbonyl]phenyl]-N′-[4-(4,6-di-4-morpholinyl-1,3,5-triazin-2-yl) phenyl]urea), PI 103 hydrochloride (3-[4-(4-Morpholinylpyrido[3′,2′: 4,5]furo[3,2-d]pyrimidin-2-yl]phenol hydrochloride), PI 828 (2-(4-Morpholinyl)-8-(4-aminopheny)I-4H-1-benzopyran-4-one), PP 121 (1-Cyclopentyl-3-(1H-pyrrolo[2,3-b]pyridin-5-yl)-1H-pyrazolo[3,4-d]pyrimidin-4-amine), Quercetin, TG 100713 (3-(2,4-Diamino-6-pteridinyl)-phenol), Wortmannin, PIK90, GDC-0941, antibodies to PI3K and PI3K receptors, PI3K inhibitory nucleic acids, and the like.

In some instances, factor useful in a particular composition may include an activator or inhibitor of the PDGF pathway. Activators and inhibitors of the PDGF pathway include small molecule activators, small molecule inhibitors, peptide activators, peptide inhibitors, antibodies, nucleic acid activators, nucleic acid inhibitors, and the like that activate or inhibit at least one component of the PDGF pathway resulting in a corresponding activation or inhibition in cellular PDGF signaling. Components and downstream effectors of the PDGF pathway include but are not limited to, e.g., 14-3-3 e (UniProtID P62258), abi1 (UniProtID Q8IZPO), acta2 (UniProtID P62736), afadin (UniProtID P55196), alpha actinin 4 (UniProtID O43707), alphav integrin (UniProtID P06756), arap1 (UniProtID Q96P48), arp2 (UniProtID P61160), arp3 (UniProtID P61158), arpc1b (UniProtID 015143), arpc2 (UniProtID O15144), arpc3 (UniProtID 015145), arpc4 (UniProtID P59998), arpc5 (UniProtID 015511), beta3 integrin (UniProtID P05106), blk (UniProtID P51451), braf (UniProtID P15056), c3g (UniProtID Q13905), c-abl (UniProtID P00519), caveolin-1 (UniProtID Q03135), caveolin-3 (UniProtID P56539), cbl (UniProtID P22681), ck2a1 (UniProtID P68400), cortactin (UniProtID Q14247), crk (UniProtID P46108), crkl (UniProtID P46109), csk (UniProtID P41240), dep1 (UniProtID Q12913), dock4 (UniProtID Q8N110), dynamin 2 (UniProtID P50570), elk1 (UniProtID P19419), eps8 (UniProtID Q12929), erk1 (UniProtID P27361), erk2 (UniProtID P28482), fgr (UniProtID P09769), fos (UniProtID P01100), fyn (UniProtID P06241), gab1 (UniProtID Q13480), grb10 (UniProtID Q13322), grb2 (UniProtID P62993), hck (UniProtID P08631), h-ras isoform 1 (UniProtID P01112), h-ras isoform 2 (UniProtID P01112), hspc300 (UniProtID Q8WUW1), ifn-gamma (UniProtID P01579), iqgap1 (UniProtID P46940), irsp53 (UniProtID Q9UQB8), jak1 (UniProtID P23458), jak2 (UniProtID O60674), jnk1 (UniProtID P45983), jnk2 (UniProtID P45984), jnk3 (UniProtID P53779), jun (UniProtID P05412), jund (UniProtID P17535), k-ras isoform 2a (UniProtID P01116-1), k-ras isoform 2b (UniProtID P01116-2), ksr (UniProtID Q8IVT5), Ick (UniProtID P06239), Irp1 (UniProtID Q07954), lyn (UniProtID P07948), mek1 (UniProtID Q02750), mek2 (UniProtID P36507), mkk4 (UniProtID P45985), mkk7 (UniProtID 014733), myc (UniProtID P01106), myocardin (UniProtID Q8IZQ8), nap1 (UniProtID Q9Y2A7), nck1 (UniProtID P16333), nck2 (UniProtID O43639), nherf1 (UniProtID 014745), nherf2 (UniProtID Q15599), n-ras (UniProtID P01111), n-wasp (UniProtID 000401), p101 (UniProtID Q8WYR1), p110-alpha (UniProtID P42336), p110-beta (UniProtID P42338), p110D (UniProtID 000329), p130 cas (UniProtID P56945), p190rhogap (UniProtID Q9NRY4), p52 shc (UniProtID P29353-2), p55-gamma (UniProtID Q92569), p62dok (UniProtID Q99704), p84 (UniProtID Q5UE93), p85-beta (UniProtID O00459), pag1 (UniProtID Q9NWQ8), pak1 (UniProtID Q13153), pdgfa (UniProtID P04085), pdgfb (UniProtID P01127), pdgfc (UniProtID Q9NRA1), pdgfd (UniProtID Q9GZPO), pdgfra (UniProtID P16234), pdgfrb (UniProtID P09619), PI3Kgamma (UniProtID P48736), pik3r1 (UniProtID P27986), pin1 (UniProtID Q13526), pkc alpha (UniProtID P17252), pkc delta (UniProtID Q05655), pkc epsilon (UniProtID Q02156), pkr (UniProtID P19525), pla2g4a (UniProtID P47712), plcgamma1 (UniProtID P19174), ppp2ca (UniProtID P67775), ppp2r1a (UniProtID P30153), ppp2r2b (UniProtID Q00005), pten (UniProtID P60484), ptp1b (UniProtID P18031), rab4a (UniProtID P20338), rab5 (UniProtID P20339), rac1 (UniProtID P63000), raf1 (UniProtID P04049), rap1a (UniProtID P62834), rap1b (UniProtID P61224), rasgap (UniProtID P20936), rhoa (UniProtID P61586), rhogdi (UniProtID P52565), rntre (UniProtID Q92738), rsk2 (UniProtID P51812), s1p1 (UniProtID P21453), shb (UniProtID Q15464), shc (UniProtID P29353), shf (UniProtID Q7M4L6), shp2 (UniProtID Q06124), slap (UniProtID Q13239), sm22 (UniProtID Q01995), sos1 (UniProtID Q07889), spa-1 (UniProtID Q96FS4), sphk1 (UniProtID Q9NYA1), sra1 (UniProtID Q96F07), src (UniProtID P12931), srf (UniProtID P11831), stat1 (UniProtID P42224), stat3 (UniProtID P40763), STAT5A (UniProtID P42229), STAT5B (UniProtID P51692), tcptp p45 (UniProtID P17706-1), vav2 (UniProtID P52735), wave2 (UniProtID Q9Y6W5), yes (UniProtID P07947), ywhab (UniProtID P31946), ywhag (UniProtID P61981), ywhah (UniProtID Q04917), ywhaq (UniProtID P27348), ywhas (UniProtID P31947), ywhaz (UniProtID P63104),, and the like.

Activators of the PDGF pathway include but are not limited to, e.g., PDGF family ligands (.e.g., PDGF, PDGF A, PDGF B, PDGF C, PDGF D, etc.) and fragments thereof and/or dimers thereof (e.g., PDGF-AA, PDGF-BB, PDGF-CC, PDGF-DD, PDGF-AB, etc.), and the like. In some instances, activation of the PDGF pathway may be achieved through repression of a PDGF pathway inhibitor, e.g., including but not limited to the use of an inhibitory nucleic acid targeting an inhibitor of the PDGF pathway or an antibody or small molecule directed to a PDGF pathway inhibitor.

Inhibitors of the PDGF pathway include but are not limited to, e.g., AG 18 ([(3,4-Dihydroxyphenyl)methylene]-propenedinitrile), AG1295, AG1296, AGL2043, AP 24534 (3-(2-Imidazo[1,2-b]pyridazin-3-ylethynyl)-4-methyl-N-[4-[(4-methyl-1-piperazinyl)methyl]-3-(trifluoromethyl)phenyl]-benzamide), CDP860, DMPQ dihydrochloride (5,7-Dimethoxy-3-(4-pyridinyl)quinoline dihydrochloride), Imatinib, PD 166285 dihydrochloride (6-(2,6-Dichlorophenyl)-2-[[4-[2-(diethylamino)ethoxy]phenyl]amino]-8-methylpyrido[2,3-d]pyrimidin-7 (8H)-one dihydrochloride), SU 16f (5-[1,2-Dihydro-2-oxo-6-phenyl-3H-indol-3-ylidene)methyl]-2,4-dimethyl-1H-pyrrole-3-propanoic acid), SU 6668 (5-[1,2-Dihydro-2-oxo-3H-indol-3-ylidene)methyl]-2,4-dimethyl-1H-pyrrole-3-propanoic acid), SU11248, Sunitinib malate (N-[2-(Diethylamino)ethyl]-5-[(Z)-(5-fluoro-1,2-dihydro-2-oxo-3H-indol-3-ylidine)methyl]-2,4-dimethyl-1H-pyrrole-3-carboxamide (2S)-2-hydroxybutanedioate salt), Toceranib (5-[(Z)-(5-Fluoro-1,2-dihydro-2-oxo-3H-indol-3-ylidene)methyl]-2,4-dimethyl-N-[2-(1-pyrrolidinyl)ethyl]-1H-pyrrole-3-carboxamide), antibodies targeting PDGF and/or PDGF receptor, PDGF inhibitory nucleic acids, and the like.

In some instances, a factor useful in a particular composition may include an activator of the NOTCH pathway. Activators of the NOTCH pathway include small molecule activators, peptide activators, antibodies against NOTCH repressors, nucleic acid activators, nucleic acid inhibitors of NOTCH repressors, and the like that activate at least one component of the NOTCH pathway resulting in a corresponding activation in cellular NOTCH signaling.

Activators of the NOTCH pathway include but are not limited to, e.g., NOTCH family ligands, including both canonical and non-canonical NOTCH family ligands, and portions or fragments thereof. Canonical and non-canonical NOTCH family ligands include but are not limited to, e.g., Delta-like ligands, Jagged ligands, homologous vertebrate proteins, and polypeptides to invertebrate NOTCH ligands (e.g., delta, serrate, LAG-2, APX-1, ARG-1, DSL-1, and the like), and the like. NOTCH ligands and methods of activating NOTCH signaling are known in the art and include, e.g., those described in D'Souza et al. (Curr Top Dev Biol. 2010; 92:73-129) Li et al. (J Biol Chem. 2008; 283(12):8046-54), the disclosures of which is incorporated herein by reference in their entirety. In some instances, activation of the NOTCH pathway may be achieved through repression of a NOTCH pathway inhibitor, e.g., including but not limited to the use of an inhibitory nucleic acid targeting an inhibitor of the NOTCH pathway or an antibody or small molecule directed to a NOTCH pathway inhibitor.

In some instances, a factor useful in a particular composition may include an activator or inhibitor of the PKA/cAMP pathway (i.e., the cAMP-dependent pathway, adenylyl cyclase pathway, PAK signaling, etc.). Activators and inhibitors of the PKA/cAMP pathway include small molecule activators, small molecule inhibitors, peptide activators, peptide inhibitors, antibodies, nucleic acid activators, nucleic acid inhibitors, and the like that activate or inhibit at least one component of the PKA/cAMP pathway resulting in a corresponding activation or inhibition in cellular PKA/cAMP signaling. Components and downstream effectors of the PKA/cAMP pathway include but are not limited to, e.g., G-protein alpha-12 family, WASF1 (WAVE1), LBC, G-protein alpha-i family, AKAP2, ATP cytosol, PDE3B, SMAD3, Androgen receptor, KDELR, AKAP7 gamma, PCTK1, 4.6.1.1, AKAP12, SMAD4, Anaphase-promoting complex (APC), GABA-A receptor beta-2 subunit, Ryanodine receptor 1, Troponin I, cardiac, AKAP8, 3.1.4.17, AKAP11, PHK beta, GABA-A receptor beta-3 subunit, PKA-cat alpha, CREB1, cAMP, G-protein alpha-s, GSK3 alpha/beta, AKAP3, Adenylate cyclase, PDK (PDPK1), GABA-A receptor beta-1 subunit, PKA-reg (cAMP-dependent), PDE4D, PKA-cat (cAMP-dependent), DARPP-32, PKA-reg type II (CAMP-dependent), NFKBIA, Meprin A, beta, AKAP82, AMP, PDE3A, PKI, PHK gamma, PDE4A, NFKBIB, PP2A regulatory, BAD, p90RSK1, G-protein alpha-13, Phospholamban, G-protein alpha-i family, RAP-1A, Adenylate cyclase type II, cAMP, G-protein beta/gamma, Calcineurin A (catalytic), PKC, Calmodulin, GSK3 alpha/beta, Adenylate cyclase type VII, Adenylate cyclase type IV, Adenylate cyclase type VIII, CREB1, ATP cytosol, Ca(‘2+) cytosol, 4.6.1.1, Ryanodine receptor 1, G-protein alpha-s, PKC-alpha, RAP-2A, CaMK IV, PHK alpha, PKA-reg (cAMP-dependent), Adenylate cyclase type III, cAMP-GEFII, Adenylate cyclase type V, LIPS, KDELR, CAMP-GEFI, Adenylate cyclase type VI, PKA-cat (CAMP-dependent), PHK gamma, CaMK II, PKC-zeta, PKC-delta, Adenylate cyclase type I, Adenylate cyclase type IX, and the like.

Activators of the PKA/cAMP pathway include but are not limited to, e.g., forskolin, dibutyryl-CAMP (bucladesine), 8-bromo-CAMP, 8-CPT-CAMP, taxol, Adenosine 3′,5′-cyclic Monophosphate, N6-Benzoyl, Adenosine 3′,5′-cyclic belinostat, 8-Chloroadenosine 3′,5′-Cyclic Monophosphate, (S)-Adenosine, cyclic 3′,5′-(hydrogenphosphorothioate), Sp-Adenosine 3′,5′-cyclic monophosphorothioate, Sp-5,6-DCI-cBiMPS, Adenosine 3′,5′-cyclic Monophosphorothioate,8-Bromo-, Sp-Isomer, Sp-8-pCPT-cyclic

GMPS Sodium, N6-Monobutyryladenosine 3′:5′-cyclic monophosphate, 8-PIP-CAMP, Sp-CAMPS caffeine, theophylline, pertussis toxin and the like. In some instances, activation of the PKA/CAMP pathway may be achieved through repression of a PKA/cAMP pathway inhibitor, e.g., including but not limited to the use of an inhibitory nucleic acid targeting an inhibitor of the PKA/cAMP pathway or an antibody or small molecule directed to a PKA/cAMP pathway inhibitor.

In some instances, a factor useful in a particular induction composition may include an activator or inhibitor of the VEGF pathway. Activators and inhibitors of the VEGF pathway include small molecule activators, small molecule inhibitors, peptide activators, peptide inhibitors, antibodies, nucleic acid activators, nucleic acid inhibitors, and the like that activate or inhibit at least one component of the VEGF pathway resulting in a corresponding activation or inhibition in cellular VEGF signaling. Components and downstream effectors of the VEGF pathway include but are not limited to, e.g., VEGFA, KDR, SH2D2A, PLCG1, PLCG2, PRKCA, PRKCB, PRKCG, SPHK1, SPHK2, HRAS, KRAS, NRAS, RAF1, MAP2K1, MAP2K2, MAPK1, MAPK3, PLA2G4E, PLA2G4A, JMJD7-PLA2G4B, PLA2G4B, PLA2G4C, PLA2G4D, PLA2G4F, PPP3CA, PPP3CB, PPP3CC, PPP3R1, PPP3R2, NFATC2, PTGS2, PTK2, SHC2, PXN, CDC42, MAPK11, MAPK12, MAPK13, MAPK14, MAPKAPK2, MAPKAPK3, HSPB1, SRC, PIK3CA, PIK3CD, PIK3CB, PIK3CG, PIK3R1, PIK3R5, PIK3R2, PIK3R3, RAC1, RAC2, RAC3, AKT1, AKT2, AKT3, NOS3, CASP9, BAD, and the like.

Modulators of the VEGF signaling pathway include but are not limited to e.g., Aspirin, Naproxen, Sulindac, Ibuprofen, Piroxicam, Diflunisal, Ketoprofen, Indometacin, Mefenamic acid, Tolmetin sodium, Meclofenamate sodium, Etodolac, Flurbiprofen, Nabumetone, Sasapyrine, Oxaprozin, Phenylbutazone, Sodium salicylate, Celecoxib, Rofecoxib, Axitinib, Bosutinib, Dasatinib, Doramapimod, Pegaptanib sodium, Ranibizumab, Semaxanib, Sorafenib tosilate, Vatalanib, Sunitinib malate, Vandetanib, Bevacizumab, Dasatinib hydrate, Motesanib, Dexketoprofen, Ketoprofen sodium, Meclofenamate sodium, Piketoprofen, Piketoprofen hydrochloride, Toceranib, Sorafenib, Toceranib phosphate, Sunitinib, Bevasiranib sodium, Brivanib alaninate, Cediranib, Cediranib maleate, Motesanib phosphate, Pamapimod, Ramucirumab, Talmapimod, Aflibercept, Dilmapimod, Dilmapimod tosylate, Foretinib, Linifanib, Losmapimod, Saracatinib, Saracatinib difumarate, Tivozanib, Bosutinib hydrate, Pegdinetanib, Naproxen etemesil, Cabozantinib, Tivozanib hydrochloride, Golvatinib, Pimasertib, Pimasertib hydrochloride, and the like. In some instances, VEGF activators useful in the subject methods include but are not limited to e.g., a VEGF polypeptide and/or a nucleic acid encoding a VEGF polypeptide.

Angiopoietin activating agents include Angiopoietin 1-4 (Ang1-4) and represent an important family of growth factors, whose activities are mediated through the tyrosine kinase receptors, Tie1 and Tie2. Ang1 is a potent angiogenic growth factor signaling through Tie2, whereas Ang2 was initially identified as a vascular disruptive agent with antagonistic activity through the same receptor.

### Markers

Aspects of the present disclosure include identifying cells based on the presence or absence or relative amount of one or more markers. In some instances, markers of interest include cell surface markers that may be detected, e.g., on live cells. In other instances, markers of interest include expression markers, e.g., cellular expression markers indicative of cell type.

Markers may be detected or measured by any convenient means as such marker detection is well-known in the art and may make use of one or more detection reagents including but not limited to, e.g., antibodies, antibody fragments, binding partners (e.g., ligands, binding pairs, etc), hybridizable nucleic acids, aptamers, etc. In some instances, a marker may be a cell surface marker and detection of the marker may be performed based on the use of one or more detection reagents that specifically bind to the marker. Detection reagents, e.g., antibodies, may be detectably labeled (e.g., fluorescently labeled through the attachment of a fluorescent molecule, fluorescent bead, or other fluorescent label) or may be detected through the use of a second detectably labeled detection reagent that specifically binds to the first detection reagent (e.g., a fluorescently labeled secondary antibody). In some instances, a detection agent, e.g., having a detectable label or having been bound by a second agent having a detectable label, can be visualized or otherwise observed or detected based on the visual characteristics of the label, including e.g., fluorescent detection, colorimetric detection, and the like. Detectable labels useful in detection reagents need not be visually detectable and may, in some instances, be detected by a detection device configured to detect a non-visual detectable label including but not limited to, e.g., a magnetic label, a radioactive label, etc. In some instances, detectable labels may be detected through the use of one or more detection reactions, including but not limited to, e.g., enzymatic detection reactions (enzymatic reactions generating a detectable substrate, e.g., a fluorescent or colorimetric substrate), amplification reactions (PCR amplification, fluorescent signal amplification (e.g., tyramide signal amplification, etc.), etc.)

In certain embodiments, derived cardiac mesoderm cells may be identified or isolated based on the detection or measurement of the cell surface marker GARP (i.e. LRRC32), e.g., identified or isolated based on being positive for GARP, having a high level of GARP or having a level of GARP above a threshold level.

In certain embodiments, derived cardiac mesoderm cells may be identified or isolated based on the detection or measurement of the cell surface marker CD143, e.g., identified or isolated based on being positive for CD143, having a high level of CD143 or having a level of CD143 above a threshold level.

In certain embodiments, derived cardiac mesoderm cells may be identified or isolated based on the detection or measurement of the cell surface marker TIE2, e.g., identified or isolated based on being positive for TIE2, having a high level of TIE2 having a level of TIE2 above a threshold level.

In certain embodiments, derived cardiac mesoderm cells may be identified or isolated based on the detection or measurement of the cell surface marker CD1d, e.g., identified or isolated based on being positive for CD1d, having a high level of CD1d having a level of CD1d above a threshold level.

In some instances, identification and/or selection for sorting of cells may be performed using a combination of markers. Such combinations may include but combinations of positive selection markers, combinations of negative selection markers or mixed combinations of positive and negative selection markers.

In certain embodiments marker detection and/or measurement of marker level is performed using flow cytometry. Flow cytometry is a technique for counting, examining, and sorting microscopic particles suspended in a stream of fluid. It allows simultaneous multi-parametric analysis of the physical and/or chemical characteristics of single cells flowing through an optical and/or electronic detection apparatus. Fluorescence-activated cell sorting (FACS) is a specialized type of flow cytometry. FACS provides a method for sorting a heterogeneous mixture of biological cells into two or more containers, generally one cell at a time, based upon the specific light scattering and fluorescent characteristics of each cell. The flow cytometer and the FACS machine are useful scientific instruments as they provide fast, objective and quantitative recording of signals, e.g., fluorescent signals, and/or detection of cellular characteristics, e.g., size, granularity, viability, etc., from individual cells as well as physical separation of cells of particular interest. Fluorescent signals used in flow cytometry, for instance when quantifying and/or sorting cells by any marker present on or in the cell, typically are fluorescently-tagged antibody preparations or fluorescently-tagged ligands for binding to antibodies or other antigen-, epitope- or ligand-specific agent, such as with biotin/avidin binding systems or fluorescently-labeled and optionally addressable beads (e.g. microspheres or microbeads). The markers or combinations of markers detected by the optics and/or electronics of a flow cytometer vary and in some cases include but are not limited to: cell surface markers, intracellular and nuclear antigens, DNA, RNA, cell pigments, cell metabolites, protein modifications, transgenic proteins, enzymatic activity, apoptosis indicators, cell viability, cell oxidative state, etc.

Expression markers of interest may be used to identify a particular cell type or verify that a derived cell type expresses a characteristic component of the derived cell type. In some instances, detection of expression markers may allow for optimization of a particular differentiation protocol, e.g., to optimize production of a desired cell type based on detection of one or more expression markers. Expression markers will vary depending on the type of cell to be identified or verified and/or desired downstream uses of the cell following identification or verification with the expression marker. Types of expression markers will include but are not limited to, e.g., gene expression marker, protein expression markers, expressed reporters, and the like. Expression marker detection and/or measurement may be detrimental to cell viability (e.g., wherein detection requires lysing or fixing a cell of interest) or may be essentially neutral to cell viability (e.g., wherein detection does not require lysing or fixing a cell of interest and may be performed on live cells).

Gene expression markers include but are not limited to the presence, absence, and/or relative amounts of a particular gene transcript that is indicative of particular cell type. Protein expression markers include but are not limited to the presence, absence, and/or relative amounts of a particular expression product that is indicative of particular cell type. Protein expression markers may be intercellular proteins, intracellular proteins or cell surface proteins. In some instances, a gene expression marker and a protein expression marker derived from the same gene may be indicative of a particular cell type.

Methods of detecting and/or measuring gene expression and/or protein expression are well-known in the art and include but are not limited to, e.g., Northern blot, Western blot, ELISA, PCR, quantitative PCR, in situ hybridization, fluorescent in situ hybridization, immunohistochemistry, immunofluorescence, microarray, quantitative sequencing, RNAseq (including bulk, single cell, and single nuclear), quantitative mass spectrometry, and the like. In addition, methods of detecting and/or measuring epigenome changes are well-known in the art and include but are not limited ATACseq (both bulk and single cell).

Gene and protein expression markers useful in characterizing and/or identifying cardiac mesoderm cells, e.g., derived as described herein, include but are not limited to, e.g., NKX2.5, TBX20, HAND1, ISL1, FOXF1, and the like. In some instances, the measurement of one or more such cardiac mesoderm markers above a particular threshold is indicative of an increased likelihood that an analyzed cell or cell population is a cardiac mesoderm cell or are cardiac mesoderm cells. Generally, the detection and/or measurement of more such markers increases confidence in such determinations. In certain instances, measurement of one or more cardiac mesoderm markers above a particular threshold indicates that a cell is a cardiac mesoderm cell or a population of cells are cardiac mesoderm cells.

Gene and protein expression markers useful in characterizing and/or identifying cardiomyocyte cells, e.g., derived as described herein, include but are not limited to, e.g., CARDIAC TROPONIN (TNNT2), MYL2, MYH6, MYH7, MYL7, and the like. In some instances, the measurement of one or more such cardiomyocyte markers above a particular threshold is indicative of an increased likelihood that an analyzed cell or cell population is a cardiomyocyte cell or are cardiomyocyte cells. Generally, the detection and/or measurement of more such markers increases confidence in such determinations. In certain instances, measurement of one or more cardiomyocyte markers above a particular threshold indicates that a cell is a cardiomyocyte cell or a population of cells are cardiomyocyte cells.

Gene and protein expression markers useful in characterizing and/or identifying endocardium cells, e.g., derived as described herein, include but are not limited to, e.g., CD31, CD34, CD144, and the like. In some instances, the measurement of one or more such endocardium markers above a particular threshold is indicative of an increased likelihood that an analyzed cell or cell population is an endocardium cell or are endocardium cells. Generally, the detection and/or measurement of more such markers increases confidence in such determinations. In certain instances, measurement of one or more endocardium markers above a particular threshold indicates that a cell is an endocardium cell or a population of cells are endocardium cells.

Gene and protein expression markers useful in characterizing and/or identifying arterial endothelial cells, e.g., derived as described herein, include but are not limited to, e.g., CD31, CD34, CD144 (VE-cadherin), SCL, LMO2, FLI1, AA4.1, ESAM1, artery markers (SOX17, DLL4, JAG1, EFNB2), hemogenic markers (RUNX1, MYB), and the like. In some instances, the measurement of one or more such arterial endothelial markers above a particular threshold is indicative of an increased likelihood that an analyzed cell or cell population is an arterial endothelial cell or are arterial endothelial mesoderm cells. Generally, the detection and/or measurement of more such markers increases confidence in such determinations. In certain instances, measurement of one or more arterial endothelial markers above a particular threshold indicates that a cell is an arterial endothelial cell or a population of cells are arterial endothelial cells.

Arterial endothelial cells may also be differentiated on the basis of morphological characteristics including e.g. a distinctive network appearance. In some instances, arterial endothelial cells may also be differentiated on the basis of functional characteristics including e.g. the ability to subsequently form monocytes, the ability to subsequently form macrophages, the ability to subsequently form monocytes and macrophages, etc.

In some instances, cells may be identified based on an expressed reporter wherein the expressed reporter may be heterologous sequence introduced into a cell. For example, in some instances, heterologous sequence encoding a detectable reporter may be introduced into a cell such that upon differentiation and/or lineage restriction to a mesodermal cell type of interest the reporter, e.g., a fluorescent molecule, becomes alternatively active or inactive. As describe herein, heterologous sequence may be stably or transiently introduced. Such introduced heterologous sequence may be configured to be responsive to activation of a marker, e.g., a marker of a particular cell type as described herein or known in the art, such that upon expression of the marker the reporter is activated. Alternatively, such introduced heterologous sequence may be configured to be responsive to activation of a marker, e.g., a marker of a particular cell type as described herein or known in the art, such that the reporter is active independent of expression of the marker but upon expression of the marker the reporter is deactivated. Methods of creating and using expression reporters are well-known in art.

### Therapeutic Uses

In certain embodiments, the subject disclosure includes screening VOs derived according to the methods described herein as a method of therapy of an animal model of disease and/or a human disease. Methods of screening cells derived according to the methods described herein as a method of therapy may be, in some instances, performed according to those methods described below regarding using such cells in therapeutic protocols.

In certain embodiments, the subject disclosure includes screening VOs derived according to the methods described herein introduced to a host animal as a method of directly evaluating the cells or particular cellular behaviors, e.g., due to an introduced genetic modification or a naturally derived mutation. In one embodiment, genetically modified VOs, e.g., having at least one modified genomic locus, derived according to the methods described herein may be introduced into a host animal and the ability of the cells to differentiate into a particular tissue or cell type may be evaluated. In another embodiment, genetically modified VOs derived according to the methods described herein may be introduced into a host animal and the behavior of the cells within the host animal and/or within a tissue of the host animal may be evaluated. In another embodiment, VOs derived from a donor organism having a particular mutation or phenotype and lineage restricted according to the methods described herein may be introduced into a host animal and the behavior of the cells within the host animal and/or within a tissue of the host animal may be evaluated, including, e.g., the ability of the cells to differentiate into one or more tissue or cell types. The VOs may introduced into the host animal in an autologous graft, an allograft, or a xenograft such that the introduced cells may be derived from the host animal, a separate donor of the same species as the host animal, or a separate donor of a different species as compared to the host animal, respectively.

Aspects of the disclosure include methods for lessening the symptoms of and/or ameliorating a dysfunction. Non-limiting examples that may be subject to disease or dysfunction that may be treated according to the method described herein include but are not limited to, e.g., muscle precursor or progenitor cells, cells of the muscle, fat precursor or progenitor cells, cells of the fat, dorsal dermis precursor or progenitor cell cells, cells of the dorsal dermis, cartilage precursor or progenitor cells, cells of the cartilage, smooth muscle precursor or progenitor cells, cells of the smooth muscle, cardiomyocyte precursor or progenitor cells, cells of the heart, bone precursor or progenitor cells, cells of the bone, osteocytes, chondrocytes, cells of tendons, tenocytes, cells of skeletal muscle, cells of cardiac muscle, cells of smooth muscle, myocytes, cardiomyocytes, cells of fat, cells of brown fat, adipocytes, cells of the dermis, fibroblasts, fibrocytes, cells of the blood vessels, endothelial cells, mesangial cells, cells of the kidneys, juxtaglomerular cells, macula densa cells, podocytes, interstitial cells, cells of the blood, lymphocytes, myeloid cells, pericytes, mural cells, and the like.

Treatment methods described herein include therapeutic treatments, in which the subject is inflicted prior to administration, and prophylactic treatments, in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to mesodermal dysfunction or disorder and/or a family history indicating increased risk of mesodermal dysfunction or disorder), in which case the treatment can be a prophylactic treatment. In some embodiments, the individual to be treated is an individual with mesodermal dysfunction or disorder.

The methods of treatment described herein include administering a therapeutically effective amount of a VO to a subject in need thereof in order to treat the subject for a dysfunction or deficiency. The effective amount administered varies depending upon the goal of the administration, the health and physical condition of the individual to be treated, age, the taxonomic group of individual to be treated (e.g., human, non-human primate, primate, etc.), the degree of resolution desired (e.g., the amount of alleviation or reduction of symptoms), the formulation of the cell composition, the treating clinician's assessment of the medical situation, and other relevant factors.

A “therapeutically effective dose” or “therapeutic dose” is an amount sufficient to effect desired clinical results (i.e., achieve therapeutic efficacy) or reduce, alleviate, or prevent symptoms to a desired extent as determined by the patient or the clinician. A therapeutically effective dose can be administered in one or more administrations.

In some embodiments, a therapeutically effective dose of cells (e.g., derived mesodermal cell types, etc.) is one cell or more (e.g., 1×10² or more, 5×10² or more, 1×10³ or more, 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

A therapeutically effective dose of cells may be delivered or prepared and any suitable medium, including but not limited to, e.g., those described herein. Suitable medium for the delivery of a therapeutically effective dose of cells will vary and may depend on, e.g., the type of pluripotent cells from which the effective dose of cells is derived or the type of derived cells of the effective dose. In some instances, a suitable medium may be a basal medium. “Cell medium” as used herein are not limited to liquid media may, in some instances, include non-liquid components or combinations of liquid media and non-liquid components. Non-liquid components that may find use a delivery or preparation medium include those described herein and those known in the art. In some instances, non-liquid components include natural or synthetic extra cellular matric components including but not limited to, e.g., basement membrane matrix components and the like.

In some instances, an effective dose of the cells described herein may be co-administered with one or more additional agents (e.g., prepared in a suitable medium). Additional agents useful in such co-administration include agents that improve the overall effectiveness of the effective dose of cells or decrease the dose of cells necessary to achieve an effect essentially equal to administration of an effective dose of the cells without the additional agent.

The cells may be introduced by injection, catheter, intravenous perfusion, or the like. The cells may be frozen at liquid nitrogen temperatures and stored for long periods of time, being capable of use upon thawing. Once thawed, the cells may be expanded by use of growth factors and/or feeder cells or in feeder-free conditions associated with progenitor cell proliferation and differentiation. In some instances, the cells may be administered fresh such that the cells are expanded and differentiated and administer without being frozen. In addition, tissues and organoids comprising a population of cells may be surgically implanted.

The cells and/or compositions can be supplied in the form of a pharmaceutical composition, comprising an isotonic excipient or buffer or media prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the subject methods may be autologously derived. By autologously derived it is meant that the cells are derived from the subject that is to be treated with the cells. The cells may be derived from a tissue sample obtained from the subject including but not limited to, e.g., a blood sample (e.g., a peripheral blood sample), a skin sample, a bone marrow sample, and the like. In some instances, the sample from which cells are derived may be a biopsy or swab, e.g., a biopsy or swab collected to diagnose, monitor, or otherwise evaluate the subject, e.g., diagnose the subject for a dysfunction or deficiency. In some instances, the autologous sample from which the cells are derived may be a previously collected and stored sample, e.g., a banked tissue sample, from the subject to be treated, including but not limited to e.g., banked cardiac tissue or cells, banked musculoskeletal tissue or cells, banked reproductive tissue or cells, banked skin tissue or cells, banked bone tissue or cells, banked bone marrow tissue or cells, banked vascular tissue or cells, banked umbilical cord blood tissue or cells, and the like.

In some instances, cells of the subject methods may be non-autologously derived. By non-autologously derived it is meant that the cells are not derived from the subject that is to be treated with the cells. In some instances, non-autologously derived cells may be xeno-derived (i.e., derived from a non-human animal) or allo-derived (i.e. derived from a human donor other than the subject to be treated). Non-autologously derived cells or tissue may be derived from any convenient source of cells or tissue collected by any convenient means.

Whether to use autologously derived or non-autologously derived cells may be determined according to the discretion of the subject's clinician and may depend on, e.g., the health, age, genetic predisposition or other physical state of the subject. In some instances, autologous cells may be preferred, including, e.g., to decrease the risk or immune rejection of the transplanted cells.

Methods of derivation of pluripotent progenitor cells from an autologous or non-autologous tissue useful in the methods described herein include but are not limited to, e.g., methods of embryonic stem cell derivation and methods of induced pluripotent stem cell derivation. In some instances, methods as described herein may be performed using non-autologous pluripotent progenitor cells previously derived including, e.g., those publicly or available or commercially available (e.g., from Biotime, Inc., Alameda, CA). In some instances, methods as described herein may be performed using newly derived non-autologous pluripotent progenitor cells or newly derived autologous pluripotent progenitor cells including but not limited to, e.g., newly derived embryonic stem cells (ESC) (including, e.g., those derived under xeno-free conditions as described in, e.g., Lei et al. (2007) Cell Research, 17:682-688) and newly derived induced pluripotent stem cells (iPS). General methods of inducing pluripotency to derive pluripotent progenitor cells are described in, e.g., Rodolfa K T, (2008) Inducing pluripotency, StemBook, ed. The Stem Cell Research Community, doi/10.3824/stembook. 1.22.1 and Selvaraj et al. (2010) Trends Biotechnol, 28(4)214-23, the disclosures of which are incorporated herein by reference. In some instances, pluripotent progenitor cells, e.g., iPS cells, useful in the methods described herein are derived by reprogramming and are genetically unmodified, including e.g., those derived by integration-free reprogramming methods, including but not limited to those described in Goh et al. (2013) PLOS ONE 8(11): e81622; Awe et al (2013) Stem Cell Research & Therapy, 4:87; Varga (2014) Exp Cell Res, 322(2)335-44; Jia et al. (2010) Nat Methods, 7(3):197-9; Fusaki et al. (2009) Proc Jpn Acad Ser B Phys Biol Sci. 85(8):348-62; Shao & Wu, (2010) Expert Opin Biol Ther. 10(2):231-42; the disclosures of which are incorporated herein by reference.

### Systems

Also provided are systems for use in practicing the subject methods. Systems of the subject disclosure may include a cell production system, e.g., for the micropatterning of PSC.

In some instances, the cell production system includes a cell culture chamber or cell culture vessel for the culture of desired cell types. Such cell culture chambers may be configured for the expansion of pluripotent progenitor cells and for the differentiation and/or lineage restriction of such pluripotent progenitor cells into desired cell types. The vessel may be micropatterned with a matrix. In certain embodiments, the cell culture chamber or cell culture vessel may be an open culture system, including but not limited to e.g., tissue culture dishes, tissue culture plates, tissue culture multi-well plates, tissue culture flasks, etc. In certain embodiments, the cell culture chamber or cell culture vessel may be a closed culture system, including e.g., a bioreactor, a stacked tissue culture vessel (e.g., CellSTACK Culture Chambers available from Corning, Inc. Corning, NY). In some instances, culture media and or other factors or agents may be exchanged in and out of the cell culture chamber through the use of one or more pumps (e.g., syringe pumps, peristaltic pumps, etc.) or gravity flow devices. In instances where the cells are cultured under sterile conditions the culture system may allow for the sterile exchange of culture media, e.g., through the use of sterile tubing connected, sealed, and reconnected through the use of a sterile devices, including but not limited to, e.g., a sterile tube welder and/or a sterile tube sealer. The cell culture system may be configured to control certain environmental conditions, including but not limited to e.g., temperature, humidity, light exposure, air composition (e.g., oxygen levels, carbon dioxide levels, etc.) to achieve the conditions necessary for expansion and/or differentiation of desired cell types. In some instances, the cell culture chamber may include a cell culture vessel that includes one or more patterned cell culture substrates or one or more arrays of patterned cell culture substrates as described herein.

The cell culture chamber may be configured for the production of cells for clinical use, e.g., according to current good manufacturing practice (cGMP) compliant cell culture practices, including the methods and configurations described in e.g., Fekete et al. PLOS ONE (2012) 7(8): e43255; Pham et al. (2014) J Trans Med 12:56; Gastens et al. (2007) Cell Transplant 16(7):685-96; Fernandes et al. (2013) Stem Cell Bioprocessing: For Cellular Therapy, Diagnostics and Drug Development, Burlington, Oxford: Elsevier Science: Woodhead Publishing, the disclosures of which are incorporated herein by reference.

The cell production system may, in some instances, by computer controlled and/or automated. Automated and/or computer controlled cell production systems may include a “memory” that is capable of storing information such that it is accessible and retrievable at a later time or date by a computer. Any convenient data storage structure may be chosen, based on the means used to access the stored information. In certain aspects, the information may be stored in a “permanent memory” (i.e. memory that is not erased by termination of the electrical supply to a computer or processor) or “non-permanent memory”. Computer hard-drive, CD-ROM, floppy disk, portable flash drive and DVD are all examples of permanent memory. Random Access Memory (RAM) is an example of non-permanent memory. A file in permanent memory may be editable and re-writable.

In in certain instances, a computer controlled and/or automated cell culture system may include a module or program stored in memory for production of cells according to the methods described herein. Such a module may include instructions for the administration of induction agent and/or induction compositions, e.g., at particular timing intervals or according to a particular schedule, in order to generate a desired mesodermally derived cell type. In some instances, such a computer module may further include additional modules for routine cell culture tasks including but not limited to, e.g., monitoring and record keeping, media changes, environmental monitoring, etc.

Systems of the present disclosure include components and/or devices for delivering cells produced according to the methods described herein to a subject in need thereof. For example, in some instances a system for treating a subject with a mesodermal derived tissue dysfunction or deficiency includes a cell injection system for delivering cells in a carrier, with or without optional adjuvants, to a desired injection site, including diseased tissue, adjacent to diseased tissue, and/or within, on or near a dysfunctioning organ. Such systems utilize known injection devices (e.g., including but not limited to needles, bent needles, cannulas, syringes, pumps, infusion devices, diffusion devices, etc.) and techniques (e.g., including but not limited to intramuscular injection, subcutaneous injection, device-guided injection, etc.). In some instances, a device or technique used for the delivery of a cell scaffold or other bioengineered device may be configured or adapted for use in a cell delivery system for use in delivering cells derived according to the methods described herein. In addition, tissues and organoids comprising a population of cells may be surgically implanted.

In addition to the above described components systems of the subject disclosure may include a number of additional components, such as data output devices, e.g., monitors and/or speakers, data input devices, e.g., interface ports, keyboards, etc., fluid handling components, power sources, controllers, etc.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Micropatterned Organoids Enable Modeling of the Earliest Stages of Human Cardiac Vascularization

Although model organisms have provided insight into the earliest stages of cardiac vascularization, we know very little about this process in humans, due to ethical restrictions and the technical difficulty of obtaining embryos at very early developmental stages. Here we show that spatially micropatterned human pluripotent stem cells (hPSCs) enable in vitro modeling of the earliest stages of cardiac vascularization, corresponding to the first three weeks of in vivo human development. Using four hPSC fluorescent reporters, we create 2D and 3D cardiac vascular organoids (cVOs) by identifying a combination of growth factors that simultaneously give rise to a spatially organized and branched vascular network within endocardial, myocardial, epicardial, and progenitor cells.

Using the four hPSC fluorescent reporter systems hESC-RUES-GLR, hESC-NKX2-5-eGFP, hESC-TNNT2-GFP, and hESC-3R, along with spatially micropatterned hPSCs, we create cardiac vascularized organoids (cVOs) in a repeatable and scalable fashion. Importantly, the four hPSC reporter systems enable temporal identification of germ layer, progenitor, and cardiovascular cell types in situ without disturbing the architecture or orientation of developing cVOs. Addition of a combination of growth factors to micropatterned hPSCs simultaneously generates a spatially organized and branched vascular network within endocardial, myocardial, epicardial, and progenitor cells, along with numerous extracellular matrix (ECM) proteins.

Using single-cell RNA-sequencing (scRNA-seq), we show that the cellular composition of cVOs is similar to that of a 6.5 post-conception week (PCW) human heart (Carnegie Stages 19 and 20). Furthermore, we use machine learning to characterize differences in CM and EC formation. We find that NOTCH, BMP, and VEGF pathways are upregulated in cVOs and inhibition of these pathways disrupts vascularization. Finally, using the same vascular-inducing factors to create cVOs, we produce hepatic vascularized organoids (hVOs). This suggests that there is a conserved developmental program for creating vasculature within different organ systems. In summary, our in vitro model provides a significant technical advance for addressing questions regarding organ vascularization.

### Results

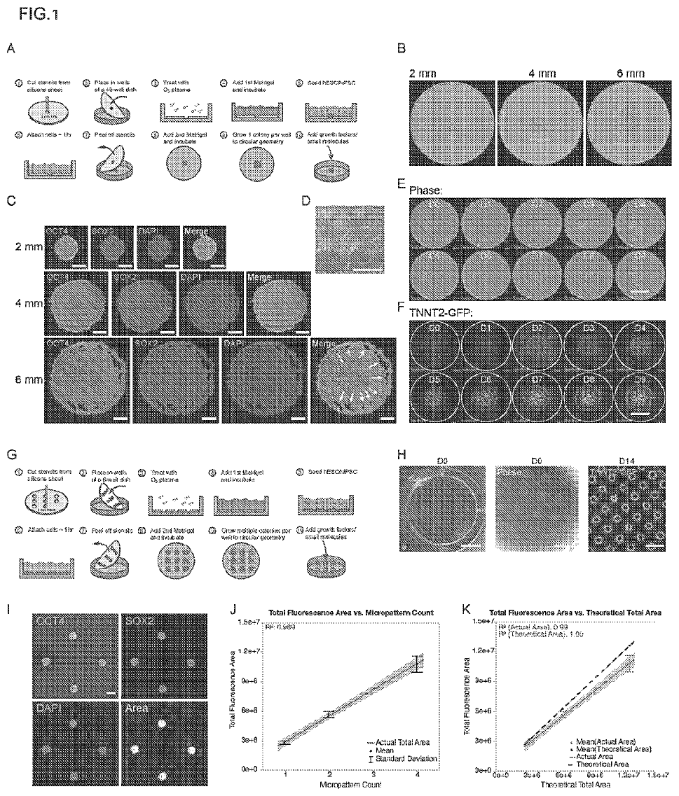

Micropatterning of hPSCs results in spatially organized cardiomyocytes. Geometric micropatterning of human pluripotent stem cells (hPSCs) enables repeatable and scalable formation of spatially organized germ layers (endoderm, mesoderm, and ectoderm), cardiomyocytes, and cardiac organoids. In each well of a multi-well plate, a single plasma-treated rubber stencil with a central hole was used to create a 2 mm circular hPSC micropattern for cardiovascular differentiation. When all the hPSC micropatterns in a plate were essentially 100% confluent, typically 2-3 days after cell seeding, differentiation was begun (FIG. 1A). Phase (FIG. 1B and S1A) and confocal fluorescence imaging (FIG. 1C) shows hPSC micropatterns were pluripotent throughout a confluent colony. However, there were areas with incomplete hESC confluence in the 6 mm colonies (FIG. 1C). For a sense of scale, a photograph shows 2 mm hPSC micropatterns (blue) in the centers of four wells of a 48-well dish (FIG. 1D). Using a hESC-TNNT2-GFP fluorescent reporter line, we show that 6 mm micropatterns could be differentiated into CMs (FIG. 1E-F) and the progression of this differentiation could be tracked in situ over days 0-9.

To scale up micropattern formation, a single plasma-treated rubber stencil with an array of 2 mm holes was used to create arrayed hPSC micropatterns. For a 6-well dish, up to 77 micropatterns (7×7 array+7×2 columns+7'2 rows) could be created per well for a total of 462 micropatterns resulting in 462 GFP+ CM colonies (FIG. 1G-H). Thus, spatial micropatterning of hPSCs produces spatially organized cardiomyocytes in single and arrayed colony formats. We confirmed that the total fluorescence area of pluripotency markers of our micropatterns was highly correlated with micropattern count (R²=0.989) and micropattern theoretical total area (R²=0.99) (FIG. 1I-K). Validation of our total fluorescence area quantification method was important for our screening experiments described below.

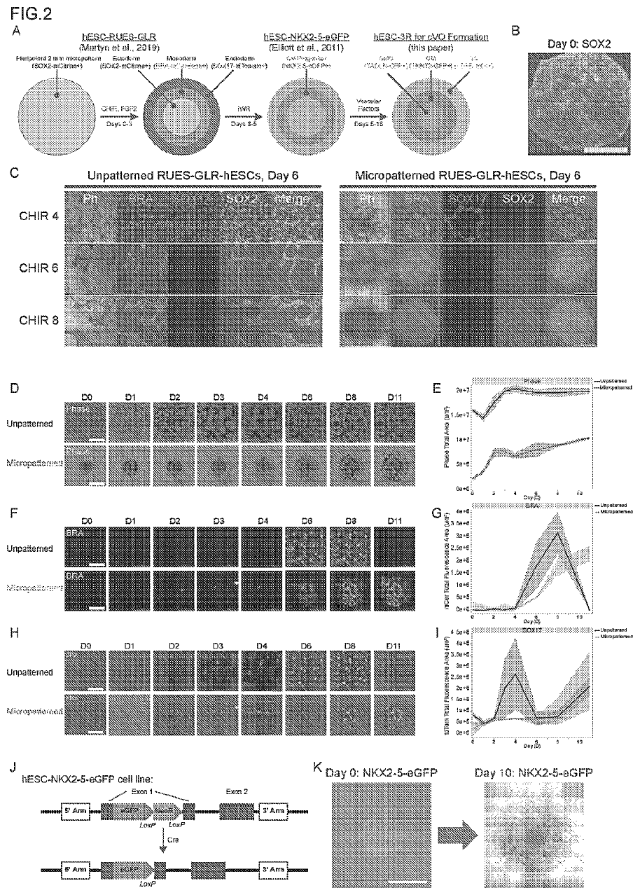

Micropatterning gives rise to organized germ layer and cardiovascular progenitor formation. To vascularize cardiac organoids, we reasoned that we would only need to induce mesoderm from our hPSC micropatterns to simultaneously produce mesoderm-derived CMs, ECs, and SMCs as previously described. However, to vascularize endoderm-derived hepatic organoids, we would need to simultaneously induce both endoderm to produce hepatocytes and mesoderm to produce ECs and SMCs. With this in mind, we used the hESC-RUES-GLR (germ layer reporter) cell line to visualize germ layer formation under different CHIR-99021 (CHIR) concentrations and unpatterned/micropatterned conditions. This line comprises SOX2 driving mCitrine to visualize pluripotency and ectoderm formation, BRA driving mCerulean expression to visualize mesoderm formation, and SOX17 driving tdTomato expression to visualize endoderm formation (and later arterial formation), and (FIGS. 2A, 2B, and 9A).

FIG. 2C shows that unpatterned RUES-GLR hESCs at day 6 gave rise to disorganized germ layer formation at different CHIR concentrations, while the micropatterned group gave rise to organized germ layer formation. For CHIR 4 uM, a central BRA+ mesodermal area was surrounded by a SOX17+ endodermal ring and no SOX2 ectodermal expression. In contrast, SOX2 was expressed in the unpatterned group at all CHIR concentrations and in a disorganized fashion. Immunostaining of BRA and SOX17 confirmed the reporter expression seen with CHIR 4 uM in the micropatterns (FIG. 9B).

To visualize temporal and spatial expression of BRA and SOX17 in unpatterned (n=2) and micropatterned groups (n=2), we differentiated RUES-GLR hESCs from days 0-11 with CHIR 4 uM at day 0 and IWR at day 3. The unpatterned group showed higher starting numbers of RUES-GLR hESCs, while both groups showed a general increase in numbers over time (FIG. 2D-E). The unpatterned group showed overall higher peak BRA+ expression, while the micropatterned group showed centrally located BRA+ expression, starting between days 4 and 6 for both groups and peaking at day 11 (FIG. 2F-G). Surprisingly, BRA expression was detected later than expected in both groups, however, we speculate that this was due to using CHIR 4 uM, which is lower than we typically use for CM differentiation. In addition, the unpatterned group showed higher and earlier SOX17+ expression (peaking at day 4), while the micropatterned group initially showed centrally located SOX17+ expression, then robust peripheral expression surrounding BRA expression, peaking at day 11 (FIG. 2H-I). Furthermore, in the unpatterned group, SOX17 expression preceded BRA expression, while in the micropatterned group, SOX17 and BRA expression occurred concurrently, with a rise in expression for both around day 6. Arrays of micropatterned RUES-GLR hESCs differentiated under the same conditions as single micropatterns also showed centrally located BRA+ expression surrounded by maximum SOX17+ expression, with peak expression for both at day 11 (FIG. 9C-D). Thus, single and arrayed micropatterns gave rise to similar BRA and SOX17 temporal and spatial expression, but different from the unpatterned group.

To visualize the effects of micropatterning on cardiovascular progenitor formation, we used the hESC-NKX2-5-eGFP cell line (FIG. 2J). Using our baseline CM differentiation protocol (see Methods, but without the addition of FGF2 at day 0), we showed that single NKX2-5-eGFP micropatterns differentiated over 10 days gave rise to organized ring formation of NKX2-5-eGFP+ cardiovascular progenitors, leading to beating cardiomyocytes. (FIG. 2K). At this juncture, using two fluorescence hPSC reporter lines, we demonstrated that micropatterning gives rise to organized germ layer and cardiovascular progenitor formation, thus providing a standardized method for screening conditions leading to cVO formation.

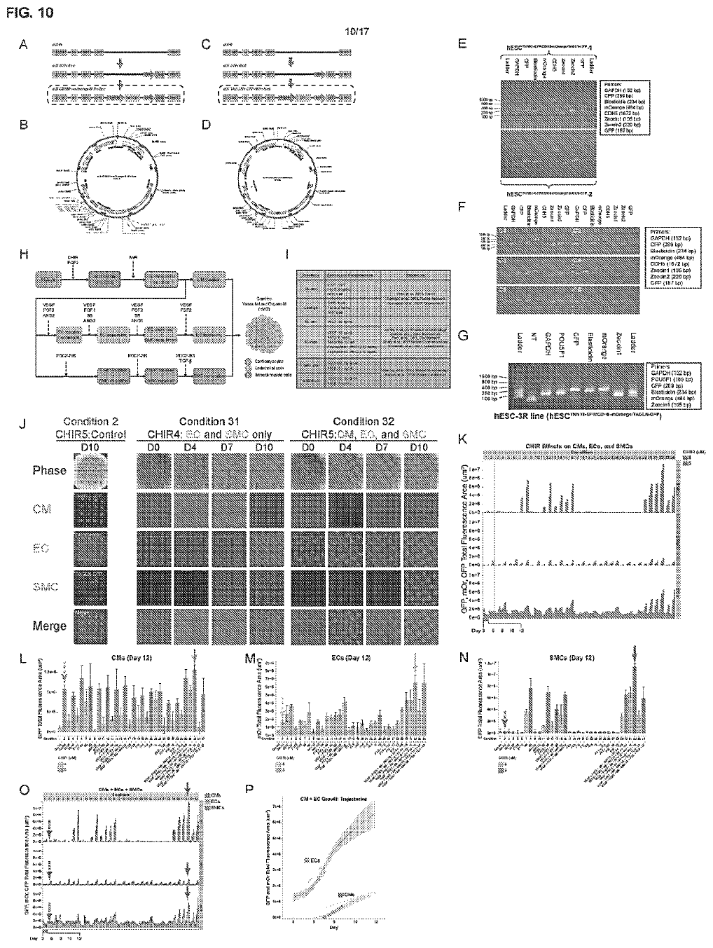

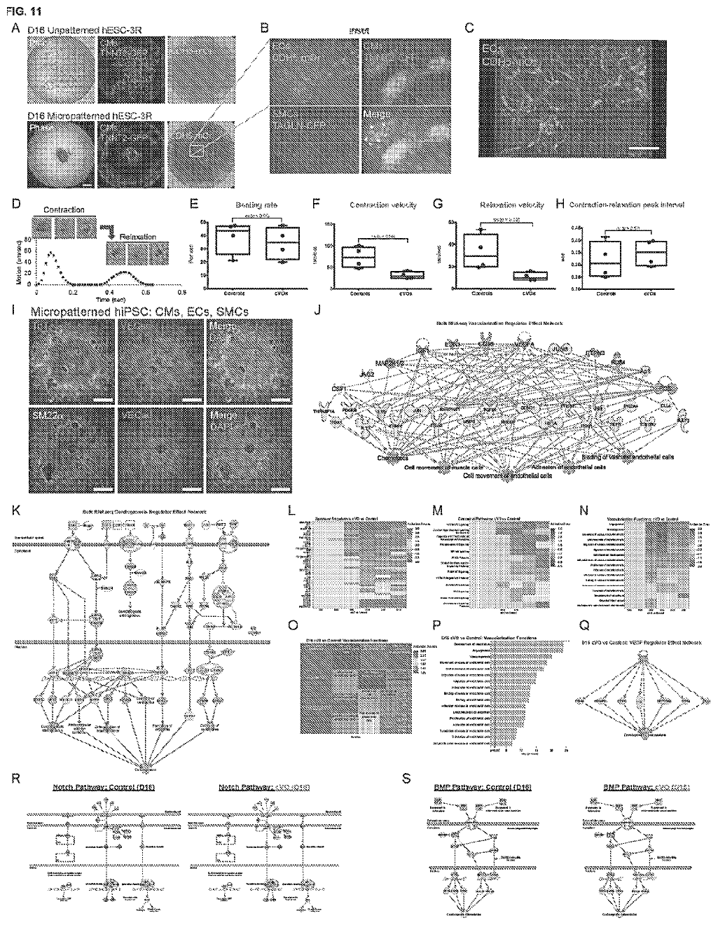

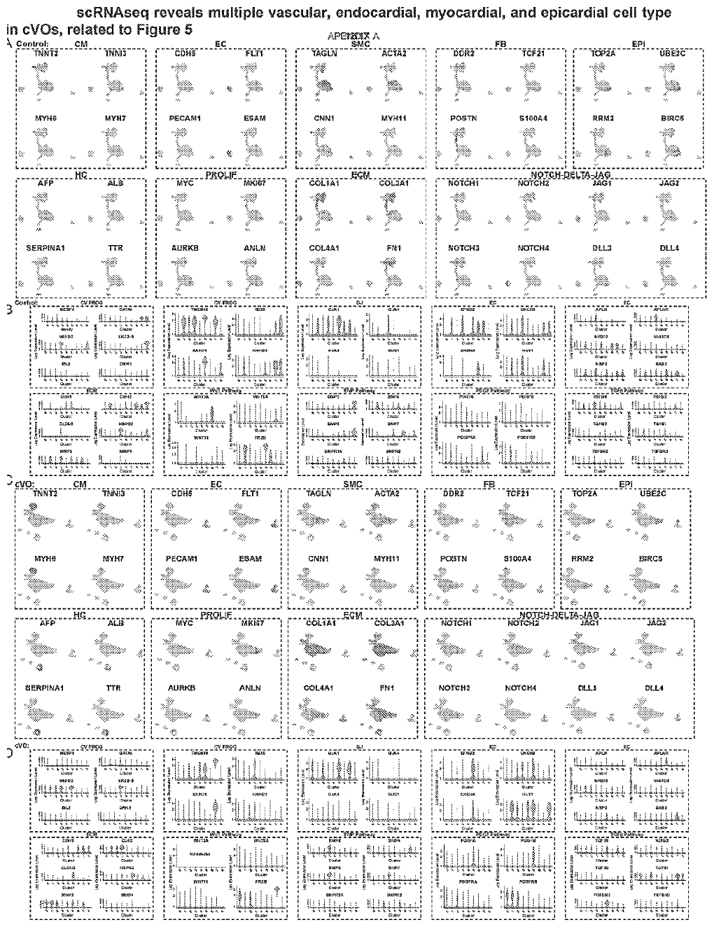

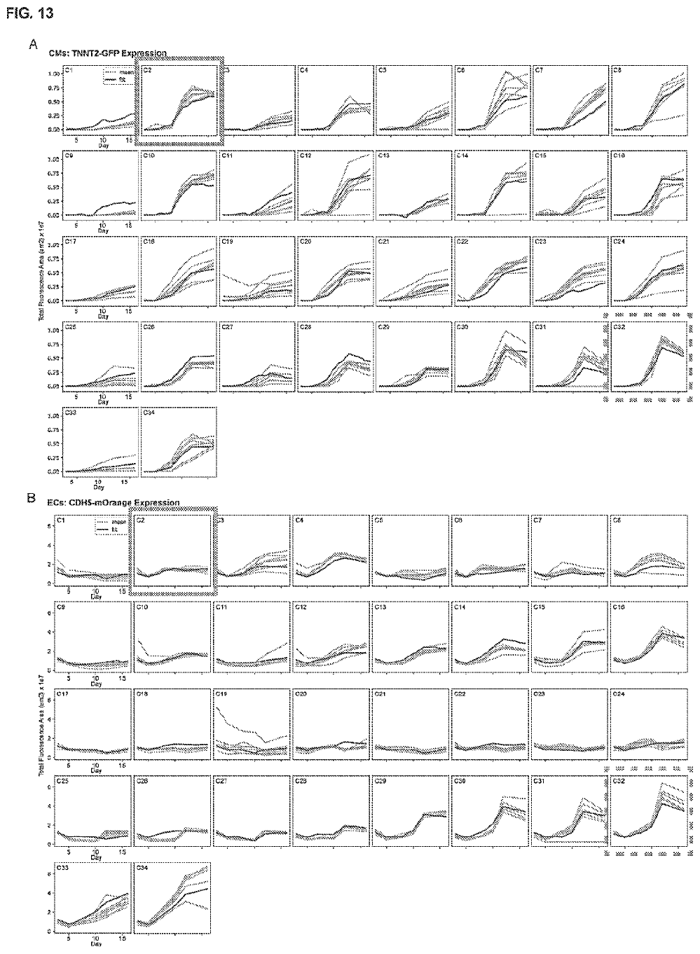

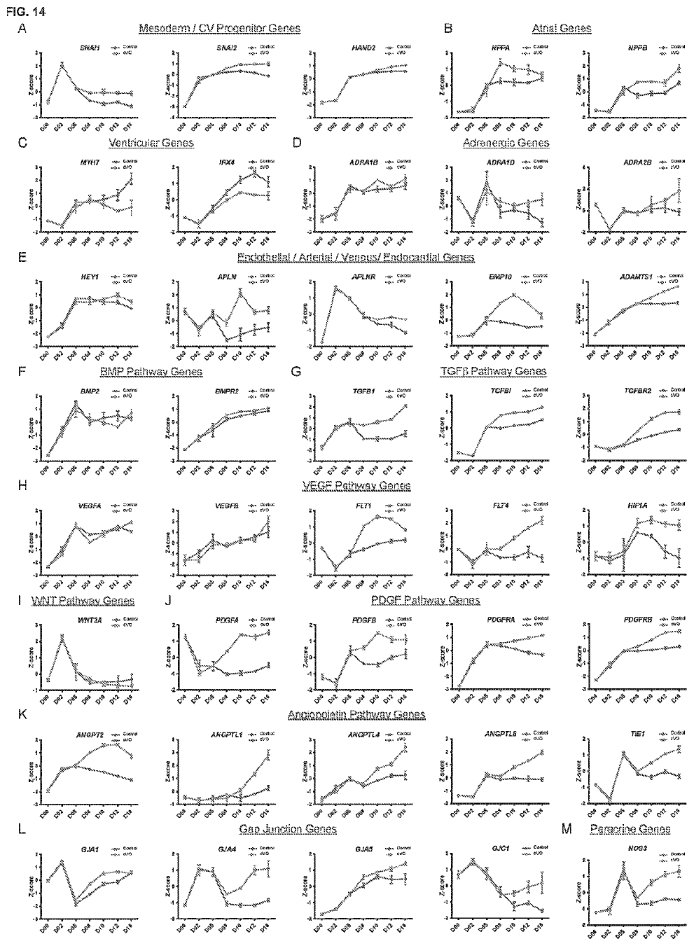

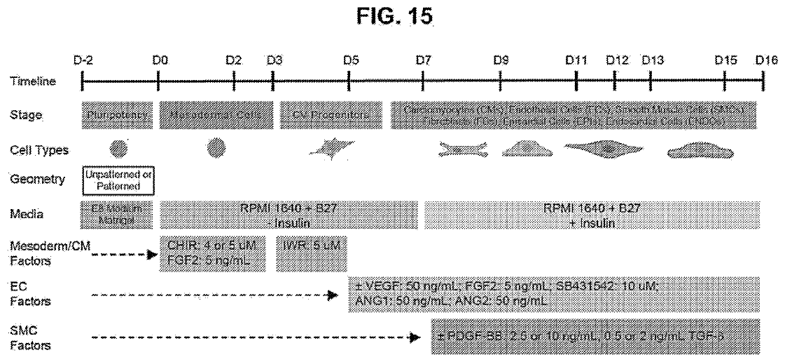

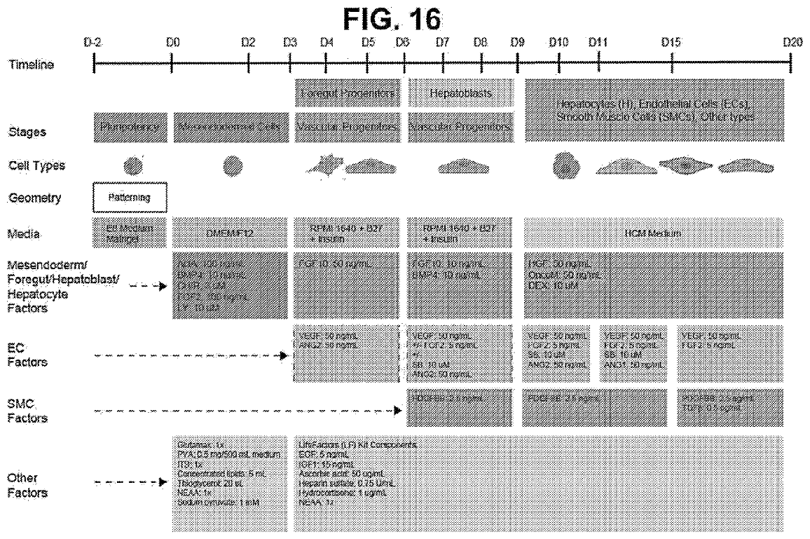

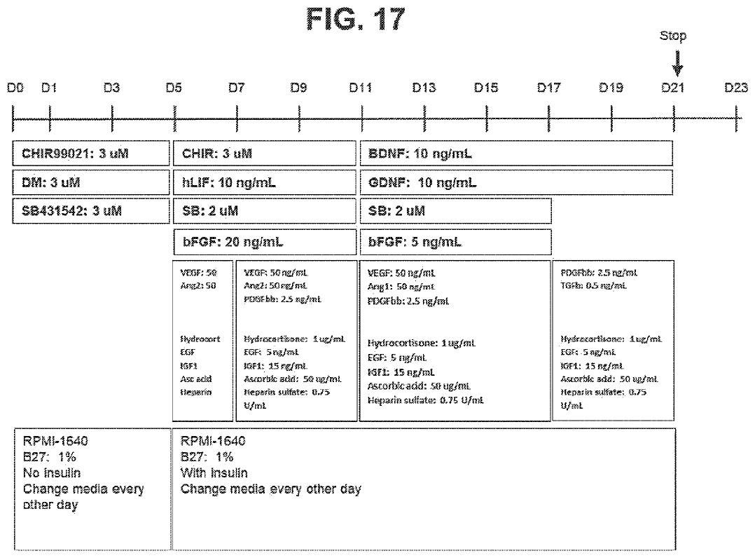

Creation of a triple reporter line enables identification of differentiation conditions leading to cVO formation. To facilitate screening differentiation conditions leading to cVO formation, we created a hESC-triple reporter line (hESC^{TNNT2-GFP/CDH5-mOrange/TAGLN-CFP}) (hESC-3R) comprising the TNNT2 promoter driving GFP to identify CMs, the CDH5 (VE-Cadherin) promoter driving mOrange to identify ECs, and the TAGLN (SM22α) promoter driving CFP to identify SMCs (FIG. 3A). Two lentiviral vectors, one for the CDH5 promoter driving mOrange (FIG. 10A-B), and the other for the TAGLN promoter driving CFP (FIG. 10C-D) were introduced into a hESC-TNNT2-GFP fluorescent reporter line (Wrighton et al., 2014). We confirmed the creation of two polyclonal lines, six monoclonal lines, and pluripotency of the monoclonal lines (FIG. 10E-G). Using separate differentiation protocols each for CMs), ECs (Paik et al., 2018), and SMCs (Cheung et al., 2012), we confirmed the differentiation of the hESC-3R line into CMs, ECs, and SMCs (FIG. 3B).

Next, we tested the effects of CHIR concentrations (3, 4, and 5 μM) and VEGF addition (50 ng/ml) on CM and EC co-differentiation over 14 days using the hESC-3R line. hESC-3R micropatterns treated with CHIR+FGF2+IWR (Control) differentiated to the most CMs with a CHIR concentration of 5 uM and a few ECs were present at all concentrations (FIG. 9E) despite no addition of VEGF. On the other hand, hESC-3R micropatterns treated with the addition of VEGF at days 6, 8, 10, and 12 differentiated simultaneously to the most CMs and ECs with CHIR 5 uM and VEGF 50 ng/ml (FIG. 9F). These initial results were the basis for our additional screening conditions described below for co-differentiating CMs, ECs, and SMCs to create cVOs.

A schematic shows timeline, stage, cell types, geometry, media, and growth factors for creating CMs, ECs, SMCs, and cVOs (FIG. 3C). Our strategy for co-differentiation consisted of first creating a cardiovascular (CV) progenitor pool that would give rise to all three cell types and then adding additional small molecules and growth factors in parallel to further co-differentiate CMs, ECs, and SMCs to produce cVOs (FIG. 10H). We used previous established methods for guiding concentrations and timing for low and high CM production, low and high EC production, and low and high SMC production (FIG. 10I).

A total of thirty-four (34) screening conditions (n=4-6 per micropatterns per condition, 175 total micropatterns) were used to create various combinations of CMs, ECs, and SMCs (Table 1). Odd numbered conditions (1-33) used CHIR 4 uM (n=88 micropatterns) and even numbered conditions (2-34) used 5 uM (n=87 micropatterns). Overall, 5 μM gave rise to the most CMs (***p<0.0001), ECs (***p<0.0001), and SMCs (***p<0.0001) compared to 4 uM for each condition (FIG. 3D, S3J-N). Condition 32 produced cVOs with the most CMs, ECs and SMCs (FIGS. 3E, 3I, 3M and S3O). Condition 32 also produced the most cell types at day 12 for even-numbered conditions using CHIR 5 uM (FIG. 3F, 3J, 3N).

For arrays of hESC-3R micropatterns, Control (Condition 2) using CHIR 5 uM gave rise to CMs, some ECs, and no SMCs. Condition 31 using the same vascular factors as Condition 32, but with CHIR 4 um gave rise to ECs and SMCs, but no CMs, while Condition 32 gave rise to all three cell types (FIG. 10J). Condition 32 consists of the following small molecules and growth factors: CHIR, 5 uM (day 0); FGF2, 5 ng/ml (days 0, 7, 9, 11, 13); IWR, 5 uM (day 3); VEGF, 50 ng/ml (days 5, 7, 9, 11, 13); SB 10 uM (days 7, 9, 11); ANG2, 50 ng/ml (days 5, 7); ANG1, 50 ng/ml (days 9, 11); PDGFBB, 10 ng/ml (days 7, 9, 11, 13); and TGFβ, 2 ng/ml (days 13). Condition 2 (Control) is the standard baseline CM differentiation with only the small molecules CHIR, FGF2, and IWR.

Time-course CM formation for Control (n=4) and Condition 32 (n=6) showed that CMs began to form around day 8 for both conditions (FIG. 3G) and by day 12, there were statistically higher CMs in Condition 32 (***p<0.0001) (FIG. 3H). Time-course EC formation showed that ECs were already apparent at day 3 for both conditions and their formation began to increase around day 5 for Condition 32, a few days before CM formation (FIG. 3K); by day 12, there were statistically higher ECs in Condition 32 (***p<0.0001) (FIG. 3L). Time-course SMC formation showed that SMC formation began to increase around day 8 for Condition 32 (FIG. 3O) and by day 12, there were statistically higher SMCs in Condition 32 (***p<0.0001) (FIG. 3P). Interestingly, at day 12, VEGF alone gave the most ECs compared to Control (n=10, ***p<0.0001), to FGF2 alone (n=10, ***p<0.0001), and to SB alone (n=8, *p<0.05), while having no statistical (ns) effect on CM formation (FIG. 3Q).

Time-lapse microscopy showed more CM, EC, and SMC formation in Condition 32 compared to Control (FIG. 3R-S). As noted above, CMs began to form around day 8 and ECs were already apparent at day 3 in both conditions; in Condition 32, the rates of EC and SMC formation increased around days 5 and 8, respectively (FIG. 10P). We also found that the total fluorescence generated by the 34 screening conditions for cVO formation were well fit by simple machine learning models (general linear), encapsulating CM (R²=0.88; FIG. 13A), and EC (R²=0.78; FIG. 13B) formation over time. In summary, creation of the hESC-3R line enabled identification of differentiation conditions leading to cVO formation.

cVOs comprise spatially and temporally self-organized cardiovascular cell types. We extended the differentiation of cVOs from 12 to 16 days (˜3 weeks of in vivo human development); these contained CMs, SMCs, and branching ECs arranged in a concentric fashion (FIG. 4A-B). We confirmed CMs co-expressed Troponin-T (TnT) and TNNT2-GFP while ECs co-expressed PECAM and CDH5-mOr (FIG. 4C-D). Compared to hESC-3R micropatterns, unpatterned hESC-3R cells resulted in unorganized and random distribution of CMs and ECs (FIG. 11A). High magnification microscopy of cVOs also showed ECs intimately surrounding CMs and moving in unison together with each CM contraction along with branching and lumen formation (FIG. 11B-C). Furthermore, we validated our vascularization protocol with a hiPSC line (SCVI 113, Stanford CVI Biobank) and confirmed the presence of CMs, ECs and SMCs within hiPSC-derived cVOs (FIG. 11I).

Arrays of hESC-3R micropatterns also differentiated into cVOs over 16 days (˜3 weeks of in vivo human development) and contained CMs, SMCs, and branching ECs arranged in a concentric fashion (FIG. 4E-F). Individual micropatterns gave rise to independent repeating patterns of cVOs despite sharing the same media. Additionally, cVOs that formed rings of CMs would beat in a rotating fashion. To create 3D cVOs we made micropatterns on a “soft” hydrogel substrate with a 16 kPa stiffness (compared to “hard” tissue culture plastic in the GPa range) which resulted in a network of ECs surrounding CMs and promoted spherical 3D formation (FIG. 4G).

In terms of function, Fluo-4 calcium CM labeling of Control and cVO groups (using hiPSCs) revealed that calcium transient rates of Controls were higher than cVOs (FIG. 4H). Additionally, the beating rate of Controls (n=15) and cVOs (n=16) significantly increased with isoproterenol (0, 1, and 10 uM) treatment (*p<0.05 and *p<0.01 at 10 uM for Control and cVO, respectively) (FIG. 4I). Based on analysis of contraction-relaxation cycles of Controls and cVOs (FIG. 11D), the beating rate, contraction velocity, and relaxation velocity of Controls (n=4) was higher than cVOs (n=4) but not statistically significant (p=0.65, 0.06, and 0.10, respectively) (FIG. 11E-S4G). The contraction-relaxation peak interval of Controls was lower than cVOs, but also not statistically significant (p=0.52) (FIG. 11H). Furthermore, EC nitric oxide (NO) secretion was significantly higher in cVO ECs compared to Controls (*p<0.05) (FIG. 4J).

We next performed temporal bulk RNA-sequencing (RNA-seq) on single undifferentiated micropatterned hESC-3R colonies, Controls, and cVOs (n=3 for all groups). Principal component analysis (PCA) showed developmental differences between all three groups over 16 days. Divergence between Control and cVO groups emerged at day 5, when vascular induction began (FIG. 4K). Weighted gene co-expression network analysis (WGCNA) showed clustering of pluripotency, mesoderm, CV progenitor, CM, EC, SMC, and vascularization genes over 16 days. Vascularization genes were highest in cVOs and peaked at day 16 (FIG. 4L).

Ingenuity Pathway Analysis (IPA) of the WGCNA Pale Turquoise module within the “Vascularization Genes” cluster in FIG. 4L confirmed a vascularization regulator effect network with upstream regulators including VEGF, CD36, JAG2, and JUNB activating genes including PDGFB, FLT1, MMP9, TGFβ1, HIF1A, FN1, and DLL4, leading to downstream vascularization functions (FIG. 11J). IPA of the WGCNA Dark Grey module within the “Cardiomyocyte/Endothelial/Smooth Muscle/Fibroblast Genes” cluster in FIG. 4L confirmed a cardiogenesis regulator effect network with upstream regulators including BMP, TGFβ, and WNT11 activating genes including MEF2C, NKX2-5, TBX5, MYH6, and NPPA leading to downstream cardiogenesis (FIG. 11K).

IPA comparing Controls to cVOs from days 2-16 showed upregulated upstream regulators known to be important in cardiovascular development, including PDGFBB, TGFβ1, CD36, BMP4, VEGFA, FGF2, and HIF1A (FIG. 11L). IPA also showed upregulated canonical pathways also known to be important in cardiovascular development, including epithelial to mesenchymal transition (EMT), HIF1α signaling, and HOTAIR signaling (FIG. 11M). Additionally, IPA showed upregulated vascularization functions including angiogenesis and vasculogenesis (FIG. 11N). Upregulation over time generally began to increase at day 8, after vascularization factors were added in culture at day 5. IPA comparing Controls to cVOs on day 16 showed upregulation of vascularization functions (FIG. 11O-S4P) and a VEGF regulator effect network on ITGA1, MMP9, CDH5, F3, NOTCH4, CD34, and DLL4 leading to downstream development of vasculature (FIG. 11Q). Furthermore, IPA showed that the NOTCH pathway was upregulated in cVOs compared to Controls (FIG. 11R), while the BMP pathway was upregulated in both groups (FIG. 11S).

From our RNA-seq analysis, we compared Control and cVO groups for select gene groups as follows: pluripotent, mesoderm/CV progenitor, CM, EC, SMC, FB, epicardial, and NOTCH-DELTA-JAG (FIG. 4M). As expected, pluripotent genes decreased and mesoderm/CV progenitor genes increased. CM genes were higher for Controls compared to cVOs. For EC and Notch-Delta-Jag genes, divergence of expression between Controls and cVOs began around day 5, when vascular differentiation factors were added to the cultures. SMC and FB genes generally increased for both groups. Interestingly, the epicardial gene WT1 was higher in cVOs than Controls, while the TOP2A, UBE2C, and RRM2 genes decreased, suggesting a decrease in proliferation of epicardial cells in cVOs.

For cVOs, we also found a multitude of upregulated genes related to vascularization in EC, arterial, venous, endocardial, TGFβ pathway, VEGF pathway, PDGF pathway, gap junction, and paracrine gene groups (FIG. 14A-M). Notably, we found that several members of the angiopoietin family ANGPT2, ANGPTL1, ANGPTL4, ANGPTL6, and TIE1 were all upregulated in cVOs, consistent with their established role in vascularization.

Finally, to demonstrate increased throughput and eliminate using stencils, we used the same differentiation protocol for 2D cVOs to create 3D cVOs over 14 days, using a range of CHIR (6.0-8.5 uM) on a single hESC-3R colony formed in each well of a 96-well plate. CHIR 7.0 μM gave the most CMs and ECs, with CM and EC formation inversely related across all concentrations (FIG. 4N). The higher CHIR needed in this format was not unexpected as we have previously needed to adjust CHIR concentrations depending on cell lines and multi-well plate format.

Single-cell RNA-sequencing reveals multiple vascular, endocardial, myocardial, and epicardial cell types in cVOs. To obtain higher cellular resolution within Control and cVO groups, we next performed single-cell RNA-sequencing (scRNA-seq) and then primary downstream analysis with Seurat. Non-linear dimensional reduction using UMAP of the Control and cVO groups showed 8 clusters containing CMs, ECs, SMCs, fibroblasts (FB), epicardial cells (EPI), precursor cells (PRE), hepatic cells (HC), neural cells (NC), proliferating cells (PROLIF), and epithelial cells (EPT) (FIG. 5A, 5C), as also identified in UMAP feature plots (FIG. 12A, 12C). Further analysis revealed composition and lineage relationships of cellular subtypes in both groups (FIG. 5B, 5D). Violin plots showed gene expression of cardiovascular progenitors (CV PROG), gap junctions (GJ), ECs, ECMs, and WNT/BMP/PDGF/TGFβ pathways (FIG. 12B, 12D). We identified cell types in cVO UMAP clusters according to scaled, log-normalized differentially expressed genes (FIG. 5E). Overall, the CM clusters in the Control group shared several genes with SMCs, EPIs, and FBs while the cVO group had one distinct CM cluster. Notably and as predicted from our imaging data, the cVO group had more ECs than Control (9 vs 7%), although based on our imaging data, we believe this is an underestimate and that ECs may have been lost during the dissociation process.

Comparison with the cellular composition of 6.5 post-conception week (PCW) (˜45 days) human heart (Carnegie Stages 19 and 20) from a recent study (Asp et al., 2019) revealed that cVOs shared 9 of 14 cell types (FIG. 5F). This is the earliest in vivo scRNA-seq dataset to date but is still ˜3.5 weeks older than our cVOs and thus we expected our cVOs would not have all the cell types found in the 6.5 PCW heart. Violin plots of cVOs showed expression of CMs, ECs, SMCs, FBs, EPIs, HCs, PROLIFs, ECM, NOTCH pathway, and VEGF pathway genes in corresponding UMAP clusters (FIG. 5G-P).

Subcluster analysis of CM cluster 3 revealed atrial (MYH6+, MYL7+) and ventricular (MYH7+, MYL2+) subtypes (FIG. 5Q). Similarly, EC cluster 5 revealed arterial (EFNB2+, SOX17+, DLL4+, UNC5B+, CXCR4+) and venous (NRP2+, TEK+, APLNR+, DAB2+, EPHB4+) subtypes. Of note, the arterial subtype expressed a subset of venous genes and vice-versa (FIG. 5R). In addition, the EC cluster 5 revealed an endocardial (CDH5+, UBE2C+, CDK1+, TOP2A+, RRM2+, KIAA0101+) subtype (FIG. 5S). Finally, cluster 2 revealed SMC (TAGLN+, ACTA2+), FB (TCF21+), and EPI (TOP2A+, RRM2+, CDK1+, UBE2C+, BIRC5+, KIAA0101+) subtypes (FIG. 5T). In summary, scRNA-seq revealed multiple vascular, endocardial, myocardial, and epicardial cell types in cVOs, further increasing the cellular subtype resolution of the CMs, ECs, and SMCs identified by fluorescence imaging and RNA-seq.

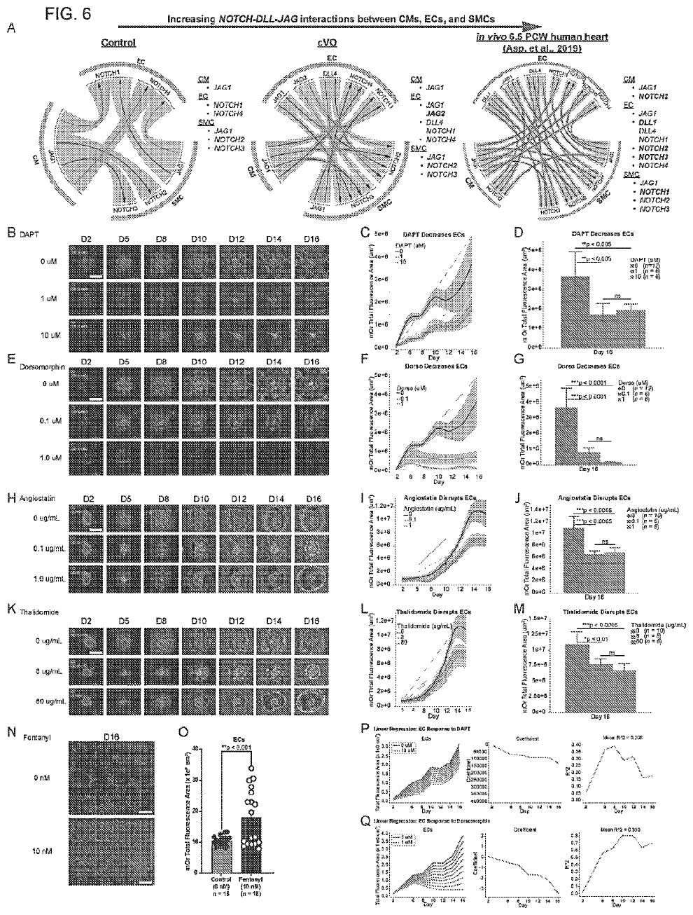

Inhibition of NOTCH, BMP, and VEGF pathways disrupts vasculature within cVOs. The NOTCH pathway is vitally important for cardiovascular development, disease, and regeneration (MacGrogan et al., 2018). Based on our RNA-seq and scRNA-seq data, members of the NOTCH pathway, including JAG1, JAG2, DLL4, NOTCH1, NOTCH2, NOTCH3, and NOTCH4, were upregulated in our cVOs (FIG. 4M, 11R, 5O, and 12C). Using our scRNA-seq data, we examined NOTCH-DLL-JAG receptor-ligand pairs between CMs, ECs, and SMCs to compare their putative cell-cell interactions in Control, cVO, and 6.5 PCW human heart groups. The Control group contained Notch receptors and Jag ligands, but no Delta ligands, on CMs, ECs, and SMCs, and interactions between these cells could be visualized by connecting their receptors to their ligands (FIG. 6A, left). The cVO group showed more receptors and ligands along with increased interactions (FIG. 6A, middle). The 6.5 PCW heart group showed the most receptors and ligands along with the most interactions (FIG. 6A, right). In summary, the number of receptors and ligands along with cell-cell interactions increased from Control, to cVO, to 6.5 PCW heart, suggesting that increased NOTCH pathway activity is associated with increased vascularization and maturation.

To ascertain the effects of interfering with the NOTCH pathway on vascular development, we next exposed cVOs to DAPT, a known NOTCH pathway antagonist. From days 2 to 16, DAPT decreased EC formation at 1 uM (n=6, **p<0.005) and 10 uM (n=6, **p<0.005) compared to 0 uM (Control) (n=12). There was no significant (ns) difference between 1 and 10 uM (FIG. 6B-D). Based on our RNA-seq and scRNA-seq data, the BMP pathway was upregulated in cVOs. To ascertain the effects of interfering with the BMP pathway on vascular development, we exposed cVOs to Dorsomorphin, a known BMP pathway antagonist. From days 2 to 16, Dorsomorphin decreased EC formation at 0.1 uM (n=6, ***p<0.0001) and 1.0 uM (n=6, ***p<0.0001) compared to 0 uM (Control) (n=12). There was no significant (ns) difference between 0.1 and 1.0 μM (FIG. 6E-G).

Based on our RNA-seq and scRNA-seq data, the VEGF pathway was also upregulated in cVOs. To ascertain the effects of interfering with the VEGF pathway on vascular development, we exposed cVOs to Angiostatin, a known VEGF pathway antagonist. From days 2 to 16, Angiostatin disrupted EC formation at 0.1 ug/mL (n=5, ***p<0.0005) and 1.0 ug/mL (n=6, ***p<0.0005) compared to 0 ug/mL (Control) (n=10). There was no significant (ns) difference between 0.1 and 1.0 ug/mL (FIG. 6H-J). At day 16 and 0.1 ug/mL, ECs congregated in the center area of starting micropatterns while at 1.0 ug/mL, ECs congregated at the periphery of starting micropatterns (FIG. 6H).

To observe the effects of Thalidomide, a known teratogen, on vascular development, we exposed this drug to cVOs. From days 2 to 16, Thalidomide disrupted EC formation at 8 μg/mL (n=5, *p<0.01) and 80 μg/ml (n=6, ***p<0.0005) compared to 0 μg/mL (Control) (n=10). There was no significant difference between 8 and 80 μg/mL (FIG. 6K-M). At day 16 and 8 μg/mL, ECs congregated in the center area of starting micropatterns, while at 80 μg/mL, ECs congregated at the periphery of starting micropatterns (FIG. 6K), similar to Angiostatin effects.

Fentanyl, a potent opioid agonist, has contributed to the opioid epidemic in the US in recent years and has the potential to be misused during pregnancy, possibly leading to congenital malformations as a teratogen. Since fentanyl has been shown to activate multiple pro-angiogenic signaling pathways, we sought to observe any similar effects on cVO vascularization. Interestingly, at day 16, fentanyl significantly increased EC formation at 10 nM (n=18, **p<0.001) compared to 0 nM (Control) (n=18) (FIG. 6N-O), an opposite finding to the four drugs above.

Finally, we fit simple machine learning models (multiple linear regression) to the changes in EC formation as caused by DAPT (R²=0.21; FIG. 6P) and Dorsomorphin (R²=0.33; FIG. 6Q). The coefficients from the fits show that the detrimental effect of DAPT on EC formation decays over time, while the detrimental effect of Dorsomorphin exponentially increases, thus resulting in increased vascular disruption compared to DAPT.

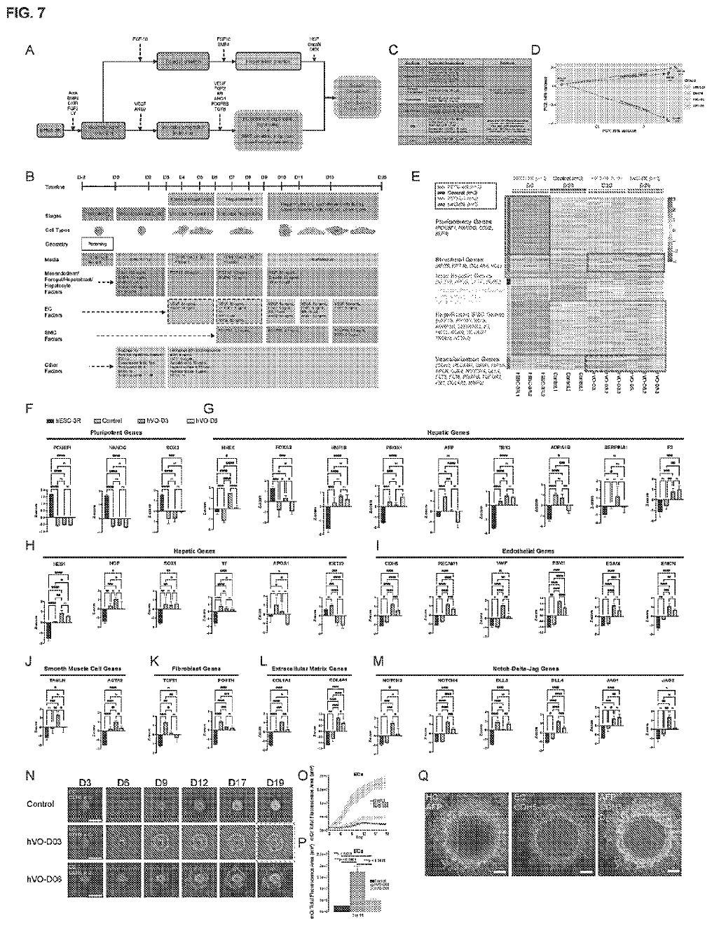

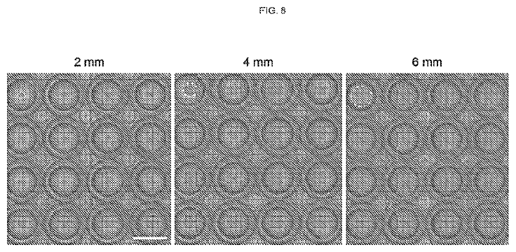

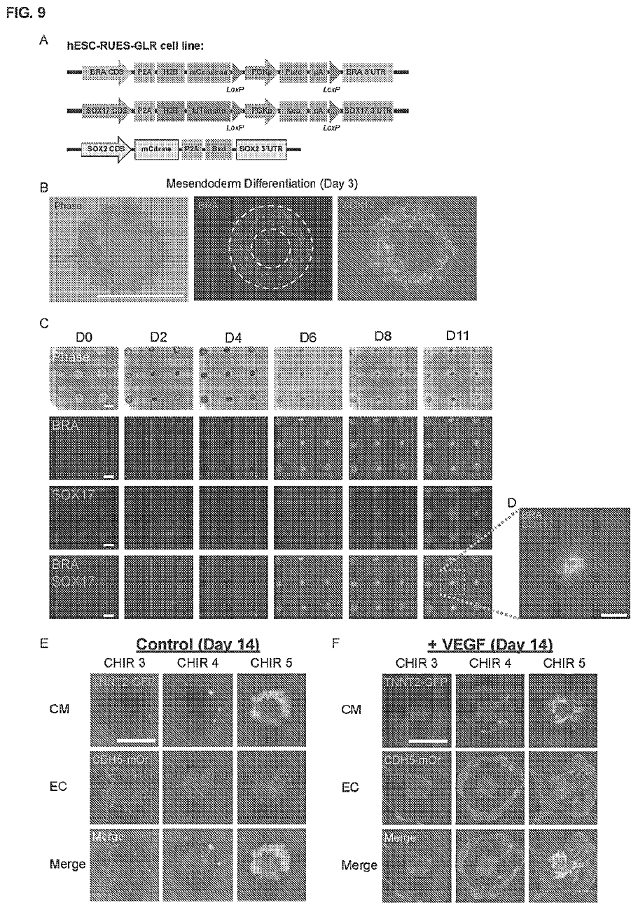

Vascularization factors used for creating cVOs enable creation of hVOs. We next asked if our vascularization strategy for cVOs could be applied to the hepatic system. Based on our results from RUES-GLR hESC micropatterning, RNA-seq, and scRNA-seq, we knew that we could produce mesendoderm, which we reasoned would be necessary for vascular and hepatic co-differentiation. Thus, our strategy for co-differentiation of ECs, SMCs, and HCs to create hVOs consisted of inducing mesendoderm and then co-differentiating a vascular progenitor pool and hepatoblast pool that would give rise to all three cell types (FIG. 7A). A schematic shows our timeline, stages, cell types, geometry, media, and growth factors for creating hVOs (FIG. 7B). We used previous established methods to guide mesendoderm, foregut progenitors, hepatoblasts, and hepatocytes (FIG. 7C). We tested 3 differentiation conditions as follows: i) Control (day 20 baseline hepatic differentiation, with no vascularization factors added); ii) hVO-D3 (day 20 hVOs created by adding vascularization factors at day 3 of differentiation); and iii) hVO-D6 (day 20 hVOs created by adding vascularization factors at day 6 of differentiation) (Table 2). We used the hESC-3R line to temporally observe vascular formation and then performed end-point immunostaining for hepatic markers; when applying our hepatic differentiation conditions, we did not observe any GFP fluorescence, thus indicating that we were not creating any “contaminating” CMs.

PCA of temporal RNA-seq showed developmental differences between hESC-3R (day 0 undifferentiated micropatterns), Control, hVO-D3, and hVO-D6 groups (n=3 for each group). A divergence between “Vascularization Factors” (D3 and D6 groups) versus “No Vascularization Factors” (Control group) was notable (FIG. 7D). WGCNA showed clustering of pluripotency, structural, miscellaneous hepatic genes, hepatic and smooth muscle cell genes, and vascularization genes. Vascularization genes were most upregulated in the hVO-D3 group (FIG. 7E). From our RNA-seq analysis, we compared the three groups for select gene groups as follows: pluripotent, HC, EC, SMC, FB, ECM, and Notch-Delta-Jag (FIG. 7F-M). Overall, pluripotent genes were upregulated for the hESC-3R group and downregulated for the Control, hVO-D3, and hVO-D6 groups. Hepatic genes were upregulated for all differentiation groups. Notably, all EC, SMC, ECM, and Notch-Delta-Jag genes (except DLL3 and JAG1) were most upregulated for the hVO-D3 group.

From days 3 to 19, time-lapse fluorescence microscopy showed EC formation for all three groups (n=10, 12, 10, respectively). Adding vascular factors at day 3 (hVO-D3 group) to the baseline hepatic differentiation protocol gave the most EC formation at day 19 (****p<0.0001) (FIG. 7N-P). Additionally, and importantly, confocal fluorescence imaging demonstrated the co-differentiation of HCs and ECs at day 19 of differentiation (FIG. 7Q). Thus, taken together, adding vascular factors at day 3 to developing hepatocytes resulted in the highest vascularization of hepatic organoids.

Spatially self-organized 2D and 3D cVOs were generated from hPSC micropatterns by identifying a combination of growth factors that simultaneously give rise to a spatially organized and branched vascular network within endocardial, myocardial, epicardial, and progenitor cells, along with numerous ECM proteins. Using scRNA-seq, we show similar cellular composition of cVOs to a 6.5 PCW human heart. Furthermore, we use machine learning to characterize differences in CM and EC formation. We find that NOTCH, BMP, and VEGF pathways are upregulated in cVOs and inhibition of these pathways disrupts vascularization. Finally, using the same vascular-inducing factors to create cVOs, we produce hVOs.

Spatial micropatterning of hPSCs enables repeatable and scalable formation of spatially organized germ layers, primitive streak, cardiomyocytes, and cardiac organoids. Herein, we combined micropatterning with four fluorescent reporter cell lines to systematically phenotype conditions leading to simultaneous co-differentiation of self-organized CMs, ECs, and SMCs. Importantly, these reporter lines enabled temporal identification of germ layer, progenitor, and cardiovascular cell types in situ without disturbing the architecture of developing cVOs.

Studies have shown the creation of 3D cardiac organoids with ECs and ENDOs lining central chambers. However, these vascular cell types were mostly found incidentally through transcriptomics and immunostaining; efforts to increase their numbers have been mostly with the addition of VEGF. In our screening conditions, we added FGF2, SB, ANG1, ANG2, PDGFBB, and TGFβ to systematically promote vasculogenesis followed by angiogenesis, and finally vessel maturation. FGF2, combined with VEGF and SB produces vascular organoids. VEGF promotes vasculogenesis, while the addition of FGF2 with VEGF promotes angiogenesis, and the addition of SB to these two factors increases hPSC-EC differentiation. Additionally, ANG2 also promotes angiogenesis, while subsequent addition of ANG1 has been shown to promote vessel maturation. Highlighting the contribution of the angiopoietins to cVO vascularization, we found that ANGPT2, ANGPTL1, ANGPTL4, ANGPTL6, and TIE1 were all upregulated in cVOs. Finally, addition of PDGFBB has been shown to recruit perivascular cells (both pericytes and SMCs) to wrap around angiogenic sprouts, and addition of TGFβ has been shown to lead to vessel maturation by inhibiting EC proliferation, stimulating SMC differentiation, and promoting ECM deposition. In support of this, we found that several members of the PDGF and TGFβ pathways were upregulated in our cVOs.

Our temporal bulk and endpoint single-cell transcriptomics revealed several cell types and pathways known to be important for cardiogenesis and vasculogenesis, including the NOTCH, BMP, and VEGF pathways. Our interference with relatively non-specific inhibitors of these pathways (DAPT, Dorsomorphin, Angiostatin) all revealed their general disruptive effects on vascularization. Our system, with both cardiac and vascular cell types, could be used to further dissect the role of NOTCH1 in hypoplastic left heart syndrome (HLHS), the role of the TGFβ pathway in left ventricular non-compaction (LVNC), and the role of the PDGF pathway in lamin A/C (LMNA) cardiomyopathy. All these pathways were upregulated in our cVOs, indicating their important role in cardiogenesis and vascularization.

Finally, using the same combination of vascular-inducing factors to create cVOs and hVOs implies a conserved developmental program for creating vasculature in different organ systems. With the RUES-GLR germ layer reporter line, various progenitor reporter lines, and parenchymal cell-specific reporter lines, our vascularization method can be added to established cell-specific differentiation protocols to achieve vascularization in other organ systems. Organoid vascularization will be necessary to i) avoid necrosis in the center of organoids where oxygen tension is low in vitro and in vivo, ii) achieve larger organoid growth in vitro for improved systems for disease modeling, drug toxicity testing, and drug discovery, and iii) increase the viability of implanted organoids for regenerative medicine applications in vivo.

To capture the benefits of micropatterning, namely, to have a reproducible and scalable system that facilitates the identification of stereotypically-formed germ layers, progenitors, and derivative cell types from hPSCs, our system produces mostly 2D cardiac organoids. The 2D system allows for easier initial phenotypic screening with conventional microscopy, especially if combined with fluorescent reporter lines; for a 3D system, dedicated confocal or light sheet microscopy would be necessary to fully characterize entire organoid and vascularization volumetric characteristics such as vessel diameter distribution, branching orders, and growth over space and time. We show that our vascularization process can be applied to a 3D system and integration with the published 3D systems could lead to recapitulating cardiogenesis with higher fidelity. The prospective and directed differentiation of vascularized organoids disclosed herein represent a significant technical advance for exploring fundamental mechanisms of vascularization, which can be applied to other organ systems for understanding human development, disease modeling, drug toxicity testing, drug discovery, and regenerative medicine.

TABLE 1
Cardiac Vascularized Organoid (cVO) Differentiation Screening Conditions
Plate 1

| Condition | | | | | | | | |
|---|---|---|---|---|---|---|---|---|
| Day | C1 | C2 | C3 | C4 | C5 | C6 | C7 | C8 |
| D0 | CHIR: 4 | CHIR: 5 | CHIR: 4 | CHIR: 5 | CHIR: 4 | CHIR: 5 | CHIR: 4 | CHIR: 5 |
| | FGF2: 5 | FGF2: 5 | FGF2: 5 | FGF2: 5 | FGF2: 5 | FGF2: 5 | FGF2: 5 | FGF2: 5 |
| D1 | RB − I | RB − I | RB − I | RB − I | RB − I | RB − I | RB − I | RB − I |
| D3 | IWR: 5 | IWR: 5 | IWR: 5 | IWR: 5 | IWR: 5 | IWR: 5 | IWR: 5 | IWR: 5 |
| D5 | RB + I | RB + I | VEGF: 50 | VEGF: 50 | Rb + I | Rb + I | Rb + I | Rb + I |
| D7 | RB + I | RB + I | VEGF: 50 | VEGF: 50 | FGF2: 5 | FGF2: 5 | SB: 10 | SB: 10 |
| D9 | RB + I | RB + I | VEGF: 50 | VEGF: 50 | FGF2: 5 | FGF2: 5 | SB: 10 | SB: 10 |
| D11 | RB + I | RB + I | VEGF: 50 | VEGF: 50 | FGF2: 5 | FGF2: 5 | SB: 10 | SB: 10 |
| D13 | RB + I | RB + I | VEGF: 50 | VEGF: 50 | FGF2: 5 | FGF2: 5 | RB+I | RB+I |
| D16 | Stop | Stop | Stop | Stop | Stop | Stop | Stop | Stop |

Units CHIR, IWR, SB: uM; Units FGF2, VEGF: ng/mL RB +/− I: RPMI-1640-B27 with/without insulin
Plate 2

| Condition | | | | | | | | |
|---|---|---|---|---|---|---|---|---|
| Day | C9 | C10 | C11 | C12 | C13 | C14 | C15 | C16 |
| D0 | CHIR: 4 | CHIR: 5 | CHIR: 4 | CHIR: 5 | CHIR: 4 | CHIR: 5 | CHIR: 4 | CHIR: 5 |
| | FGF2: 5 | FGF2: 5 | FGF2: 5 | FGF2: 5 | FGF2: 5 | FGF2: 5 | FGF2: 5 | FGF2: 5 |
| D1 | RB − I | RB − I | RB − I | RB − I | RB − I | RB − I | RB − I | RB − I |
| D3 | IWR: 5 | IWR: 5 | IWR: 5 | IWR: 5 | IWR: 5 | IWR: 5 | IWR: 5 | IWR: 5 |
| D5 | ANG2: 50 | ANG2: 50 | RB + I | Rb + I | VEGF: 50 | VEGF: 50 | VEGF: 50 | VEGF: 50 |
| | | | | | | | ANG2: 50 | ANG2: 50 |
| D7 | ANG2: 50 | ANG2: 50 | FGF2: 5 | FGF2: 5 | VEGF: 50 | VEGF: 50 | VEGF: 50 | VEGF: 50 |
| | | | SB: 10 | SB: 10 | FGF2: 5 | FGF2: 5 | FGF2: 5 | FGF2: 5 |
| | | | | | SB: 10 | SB: 10 | SB: 10 | SB: 10 |
| | | | | | | | ANG2: 50 | ANG2: 50 |
| D9 | ANG1: 50 | ANG1: 50 | FGF2: 5 | FGF2: 5 | VEGF: 50 | VEGF: 50 | VEGF: 50 | VEGF: 50 |
| | | | SB: 10 | SB: 10 | FGF2: 5 | FGF2: 5 | FGF2: 5 | FGF2: 5 |
| | | | | | SB: 10 | SB: 10 | SB: 10 | SB: 10 |
| | | | | | | | ANG1: 50 | ANG1: 50 |
| D11 | ANG1: 50 | ANG1: 50 | FGF2: 5 | FGF2: 5 | VEGF: 50 | VEGF: 50 | VEGF: 50 | VEGF: 50 |
| | | | SB: 10 | SB: 10 | FGF2: 5 | FGF2: 5 | FGF2: 5 | FGF2: 5 |
| | | | | | SB: 10 | SB: 10 | SB: 10 | SB: 10 |
| | | | | | | | ANG1: 50 | ANG1: 50 |
| D13 | RB + I | RB + I | FGF2: 5 | FGF2: 5 | VEGF: 50 | VEGF: 50 | VEGF: 50 | VEGF: 50 |
| | | | | | FGF2: 5 | FGF2: 5 | FGF2: 5 | FGF2: 5 |
| D16 | Stop | Stop | Stop | Stop | Stop | Stop | Stop | Stop |

Units CHIr, IWR, SB, uM; Units FGR2, VEGF, ANG2, ANG1: ng/mL RB +/− I: RPMI-1640-B27 with/without insulin
Plate 3

| Condition | | | | | | | | |
|---|---|---|---|---|---|---|---|---|
| Day | C17 | C18 | C19 | C20 | C21 | C22 | C23 | C24 |
| D0 | CHIR: 4 | CHIR: 5 | CHIR: 4 | CHIR: 5 | CHIR: 4 | CHIR: 5 | CHIR: 4 | CHIR: 5 |
| | FGF2: 5 | FGF2: 5 | FGF2: 5 | FGF2: 5 | FGF2: 5 | FGF2: 5 | FGF2: 5 | FGF2: 5 |
| D1 | RB − I | RB − I | RB − I | RB − I | RB − I | RB − I | RB − I | RB − I |
| D3 | IWR: 5 | IWR: 5 | IWR: 5 | IWR: 5 | IWR: 5 | IWR: 5 | IWR: 5 | IWR: 5 |
| D5 | RB + I | RB + I | RB +I | RB + I | RB + I | RB + I | RB + I | RB + I |
| D7 | PDGFBB: | PDGFBB: | PDGFBB: | PDGFBB: | TGFβ: 0.5 | TGFβ: 0.5 | TGFβ: 2 | TGFβ: 2 |
| | 2.5 | 2.5 | 10 | 10 | | | | |
| D9 | PDGFBB: | PDGFBB: | PDGFBB: | PDGFBB: | TGFβ: 0.5 | TGFβ: 0.5 | TGFβ: 2 | TGFβ: 2 |
| | 2.5 | 2.5 | 10 | 10 | | | | |
| D11 | PDGFBB: | PDGFBB: | PDGFBB: | PDGFBB: | TGFβ: 0.5 | TGFβ: 0.5 | TGFβ: 2 | TGFβ: 2 |
| | 2.5 | 2.5 | 10 | 10 | | | | |
| D13 | PDGFBB: | PDGFBB: | PDGFBB: | PDGFBB: | TGFβ: 0.5 | TGFβ: 0.5 | TGFβ: 2 | TGFβ: 2 |
| | 2.5 | 2.5 | 10 | 10 | | | | |
| | TGFβ: 0.5 | TGFβ: 0.5 | TGFß: 2 | TGFβ: 2 | | | | |
| D16 | Stop | Stop | Stop | Stop | Stop | Stop | Stop | Stop |

Units CHIR, IWR: uM; Units FGF2, PDGFBB, TGF }: ng/mL RB +/− I: RPMI-1640-B27 with/without insulin
Plate 4

| Condition | | | | | | | | |
|---|---|---|---|---|---|---|---|---|
| Day | C25 | C26 | C27 | C28 | C29 | C30 | C31 | C32 |
| D0 | CHIR: 4 | CHIR: 5 | CHIR: 4 | CHIR: 5 | CHIR: 4 | CHIR: 5 | CHIR: 4 | CHIR: 5 |
| | FGF2: 5 | FGF2: 5 | FGF2: 5 | FGF2: 5 | FGF2: 5 | FGF2: 5 | FGF2: 5 | FGF2: 5 |
| D1 | RB − I | RB − I | RB − I | RB − I | RB − I | RB − I | RB − I | RB − I |
| D3 | IWR: 5 | IWR: 5 | IWR: 5 | IWR: 5 | IWR: 5 | IWR: 5 | IWR: 5 | IWR: 5 |
| D5 | RB + I | RB + I | RB + I | RB + I | VEGF: 50 | VEGF: 50 | VEGF: 50 | VEGF: 50 |
| | | | | | ANG2: 50 | ANG2: 50 | ANG2: 50 | ANG2: 50 |
| D7 | PDGFBB: | PDGFBB: | PDGFBB: | PDGFBB: | VEGF: 50 | VEGF: 50 | VEGF: 50 | VEGF: 50 |
| | 2.5 | 2.5 | 10 | 10 | FGF2: 5 | FGF2: 5 | FGF2: 5 | FGF2: 5 |
| | TGFβ: 0.5 | TGFβ: 0.5 | TGFβ: 2 | TGFβ: 2 | SB: 10 | SB: 10 | SB: 10 | SB: 10 |
| | | | | | ANG2: 50 | ANG2: 50 | ANG2: 50 | ANG2: 50 |
| | | | | | PDGFBB: | PDGFBB: | PDGFBB: | PDGFBB: |
| | | | | | 2.5 | 2.5 | 10 | 10 |
| D9 | PDGFBB: | PDGFBB: | PDGFBB: | PDGFBB: | VEGF: 50 | VEGF: 50 | VEGF: 50 | VEGF: 50 |
| | 2.5 | 2.5 | 10 | 10 | FGF2: 5 | FGF2: 5 | FGF2: 5 | FGF2: 5 |
| | TGFβ: 0.5 | TGFβ: 0.5 | TGFβ: 2 | TGFβ: 2 | SB: 10 | SB: 10 | SB: 10 | SB: 10 |
| | | | | | ANG1: 50 | ANG1: 50 | ANG1: 50 | ANG1: 50 |
| | | | | | PDGFBB: | PDGFBB: | PDGFBB | PDGFBB: |
| | | | | | 2.5 | 2.5 | 10 | 10 |
| D11 | PDGFBB: | PDGFBB: | PDGFBB: | PDGFBB: | VEGF: 50 | VEGF: 50 | VEGF: 50 | VEGF: 50 |
| | 2.5 | 2.5 | 10 | 10 | FGF2: 5 | FGF2: 5 | FGF2: 5 | FGF2: 5 |
| | TGFβ: 0.5 | TGFβ: 0.5 | TGFβ: 2 | TGFβ: 2 | SB: 10 | SB: 10 | SB: 10 | SB: 10 |
| | | | | | ANG1: 50 | ANG1: 50 | ANG1: 50 | ANG1: 50 |
| | | | | | PDGFBB: | PDGFBB: | PDGFBB: | PDGFBB: |
| | | | | | 2.5 | 2.5 | 10 | 10 |
| D13 | PDGFBB: | PDGFBB: | PDGFBB: | PDGFBB: | VEGF: 50 | VEGF: 50 | VEGF: 50 | VEGF: 50 |
| | 2.5 | 2.5 | 10 | 10 | FGF2: 5 | FGF2: 5 | FGF2: 5 | FGF2: 5 |
| | TGFβ: 0.5 | TGFβ: 0.5 | TGFβ: 2 | TGFβ: 2 | PDGFBB: | PDGFBB: | PDGFBB: | PDGFBB: |
| | | | | | 2.5 | 2.5 | 10 | 10 |
| | | | | | TGFβ: 0.5 | TGFβ: 0.5 | TGFβ: 2 | TGFβ: 2 |
| D16 | Stop | Stop | Stop | Stop | Stop | Stop | Stop | Stop |

Units CHIR, IWR, SB: uM; Units FGF2, VEGF, ANG2, ANG1, PDGFBB, TGFβ: ng/ml RB +/− I:
RPMI-1640-B27 with/without insulin

| Plate 5 | | |
|---|---|---|
| Condition | | |
| Day | C33 | C34 |
| D0 | CHIR: 4 | CHIR: 5 |
| | FGF2: 5 | FGF2: 5 |
| D1 | RB − I | RB − I |
| D3 | IWR: 5 | IWR: 5 |
| D5 | VEGF: 50 | VEGF: 50 |
| | ANG2: 50 LF | ANG2: 50 LF |
| | Kit | Kit |
| D7 | VEGF: 50 | VEGF: 50 |
| | FGF2: 5 | FGF2: 5 |
| | SB: 10 | SB: 10 |
| | ANG2: 50 | ANG2: 50 |
| | PDGFBB: 2.5 | PDGFBB: 2.5 |
| | LF Kit | LF Kit |
| D9 | VEGF: 50 | VEGF: 50 |
| | FGF2: 5 | FGF2: 5 |
| | SB: 10 | SB: 10 |
| | ANG1: 50 | ANG1: 50 |
| | PDGFBB: 2.5 | PDGFBB: 2.5 |
| | LF Kit | LF Kit |
| D11 | VEGF: 50 | VEGF: 50 |
| | FGF2: 5 | FGF2: 5 |
| | SB: 10 | SB: 10 |
| | ANG1: 50 | ANG1: 50 |
| | PDGFBB: 2.5 | PDGFBB: 2.5 |
| | LF Kit | LF Kit |
| D13 | VEGF: 50 | VEGF: 50 |
| | FGF2: 5 | FGF2: 5 |
| | PDGFBB: 2.5 | PDGFBB: 2.5 |
| | TGF }: 0.5 | TGF }: 0.5 |
| | LF Kit | LF Kit |
| D16 | Stop | Stop |

Units CHIR, IWR, SB: uM; Units FGF2, VEGF, ANG2, ANG1, PDGFBB, TGFβ: ng/mL RB +/− I: RPMI-1640-B27 with/without insulin LifeFactors (LF) Kit: 5 ng/mL EGF, 15 ng/mL IGF-1, 50 ug/mL ascorbic acid, 0.75 U/mL heparin sulfate, 1 ug/mL hydrocortisone

TABLE 2
Hepatic Vascularized Organoid (hVOs) Differentiation Conditions
Plate 1

| Condition | 1 (Control) | 2 (“hVO-D3”) | 3 (“hVO-D6”) |
|---|---|---|---|
| D0 | ActA: 100 | Same as Control | Same as Control |
| | BMP4: 10 | | |
| | CHIR: 3 | | |
| | FGF2: 100 | | |
| | LY: 10 | | |
| D3 | FGF10: 50 | Control + | Same as Control |
| | No vascular factors | VEGF: 50 | |
| | | ANG2: 50 | |
| | | LF Kit | |
| D6 | FGF10: 50 | Control + | Control + |
| | BMP4: 10 | VEGF: 50 | VEGF: |
| | No vascular factors | FGF2: 5 | 50 |
| | | SB: 10 | ANG2: 50 |
| | | ANG2: 50 | LF Kit |
| | | PDGFBB: 2.5 | |
| | | LF Kit | |
| D9 | HGF: 50 | Control + | Control + |
| | OncoM: 50 | VEGF: 50 | VEGF: |
| | DEX: 10 | FGF2: 5 | 50 |
| | No vascular factors | SB: 10 | FGF2: 5 |
| | | ANG2: 50 | SB: 10 |
| | | PDGFBB: 2.5 | ANG2: 50 |
| | | LF Kit | PDGFBB: 2.5 |
| | | | LF Kit |
| D11 | HGF: 50 | Control + | Control + |
| | OncoM: 50 | VEGF: 50 | VEGF: |
| | DEX: 10 | FGF2: 5 | 50 |
| | No vascular factors | SB: 10 | FGF2: 5 |
| | | ANG1: 50 | SB: 10 |
| | | PDGFBB: 2.5 | ANG1: 50 |
| | | LF Kit | PDGFBB: 2.5 |
| | | | LF Kit |
| D13 | HGF: 50 | Control + | Control + |
| | OncoM: 50 | VEGF: 50 | VEGF: |
| | DEX: 10 | FGF2: 5 | 50 |
| | No vascular factors | SB: 10 | FGF2: 5 |
| | | ANG1: 50 | SB: 10 |
| | | PDGFBB: 2.5 | ANG1: 50 |
| | | LF Kit | PDGFBB: 2.5 |
| | | | LF Kit |
| D15, 17, 19 | HGF: 50 | Control + | Control + |
| | OncoM: 50 | VEGF: 50 | VEGF: |
| | DEX: 10 | FGF2: 5 | 50 |
| | No vascular factors | PDGFBB: 2.5 | FGF2: 5 |
| | | TGF [symbol] : 0.5 | PDGFBB: 2.5 |
| | | LF Kit | TGF [symbol] : 0.5 |
| | | | LF Kit 0.5 |
| D20 | Stop | Stop | Stop |

Units LY, CHIR, DEX, SB: μM Units ActA, BMP4, FGF2, FGF10, HGF, OncoM, VEGF, ANG2, ANG1, PDGFBB, TGFβ: ng/mL, LifeFactors (LF) Kit: 5 ng/mL EGF, 15 ng/mL IGF-1, 50 μg/mL ascorbic acid, 0.75 U/mL heparin sulfate, 1 μg/mL hydrocortisone

TABLE 3
PCR Primers

| | | | | | |
|---|---|---|---|---|---|
| | NLM | NLM | | | |
| Common Gene | Gene | Ascension | | | Length |
| Name | Name | Code | Forward Primer | Reverse Primer | (bp) |

PCR Primers for Lentiviral Plasmids Creation

| | | | | | |
|---|---|---|---|---|---|
| Adaptor-A/B | N/A | N/A | CTCTCTCGAGCGAGC | AATTACCCGGGATAT | N/A |
| | | | GGCCGCTCTCTAGAT | CTAGAGAGCGGCCGC | |
| | | | ATCCCGGGT | TCGCTCGAGAGAGGG | |
| | | | | CC | |
| SVPr-F/R | N/A | N/A | CTCTCTAGATATAGCA | GTGAGTTTGGCCATA | N/A |
| | | | TTGAAAAAGGAAG | CCGGTCCGCCGAGAC | |
| | | | | AGCAA | |
| Zeo-F/R | N/A | N/A | ACCGGTATGGCCAAA | GTCCCCGGGACTAGT | N/A |
| | | | CTCACTTCTGCA | CCTGTTCCT | |
| Bsd-F/R | N/A | N/A | CGGACCGGTATGCCC | GCTCCCGGGTCAGCC | N/A |
| | | | CTGAGCCAGGA | CTCCCACACA | |
| VmO-F/R | N/A | N/A | TCTCTCGAGATCGATG | ATATCTAGACTTACTT | N/A |
| | | | CTCATCCATGC | GTACAGCTCGTCCAT | |
| | | | | G | |
| TGN-F/R | N/A | N/A | AGCCTCGAGTCCGGA | GCGGTACCAAGCTTC | N/A |
| | | | TCTTGGAA | TGACTGAGAGGGTGG | |
| | | | | GTTTCCCTAGCCAG | |
| CFP-F/R | N/A | N/A | GTCAGAAGCTTGGTA | ATATCTAGATTAGCG | N/A |
| | | | CCGC | GTACAGCTCGTCCAT G | |

PCR Primers for Reporter Cell Line Creation

| | | | | | |
|---|---|---|---|---|---|
| GAPDH | GAPDH | NM002046 | GGACTCATGACCACA | TCAGGGATGACCTTG | 152 |
| (GAPDH) | | | GTCCATGCC | CCCACAG | |
| OCT-4 | POU5F1 | BC117437 | CTTGCTGCAGAAGTG | CTGCAGTGTGGGTTTC | 169 |
| (POU5F1) | | | GGTGGAGGAA | GGGCA | |
| Green | GFP | — | ACGTAAACGGCCACA | AAGTCGTGCTGCTTCA | 187 |
| Fluorescent | | | | | |
| Protein | | | AGTTC | TGTG | |
| (GFP) | | | | | |
| CDH5 (VE- | CDH5 | NM001795.5 | CCAACGGAACAGAAA | AATCCAGAGGTTGAT | 1672 |
| Cadherin) | | | CATCC | TGTCG | |
| Promoter- | | | | | |
| mOrange | | | | | |
| (CDH5) | | | | | |
| mOrange | mOrange | — | AGGGCTTTCAGACCG | TCCAACTTGATGCCG | 484 |
| (mOrange) | | | CTAAG | ACGAT | |
| Zeocin-1 | ZEO | — | GGGCTGTAGAGTTCT | CTGCTGAGATGAAGA | 105 |
| (7eocin1) | | | GGACTGA | GGGTGA | |
| Zeocin-2 | ZEO | — | CCGGTATGGCCAAAC | TCTAGGCCTCTGACCC | 220 |
| (Zeocin2) | | | TCACT | AGAC | |
| Cyan | CFP | — | TCCAGTGCTTCGCCCG | TTCGCCTCCAGGCCGT | 289 |
| Fluorescent | | | CTAC | TGTT | |
| Protein | | | | | |
| (CFP) | | | | | |
| Blasticidin | BSD | — | TAGCCGCAAACATAG | TGTTCCCAGCACCACC | 234 |
| (Blasticidin) | | | TTCAATACA | AGTT | |

### Methods

All experiments, methods, and protocols for this study were approved by the Stanford University Stem Cell Research Oversight (SCRO) committee.

Data and Code Availability. Bulk RNA-sequencing and single-cell RNA-sequencing data sets generated for this study are deposited in the Gene Expression Omnibus (GEO) under accession number[GSE185194].

Cell Lines. We used the following hPSC (both hESC and hiPSC) lines. The human H9 WT hESC (WA09, WiCell) was previously used as the parental line to create the human H9 hESC-NKX2-5-eGFP line (Elliott et al., 2011) and the human H9 hESC-TNNT2-GFP line (H9-hTnnT2-pGZ-TD2, WiCell (Wrighton et al., 2014). In our study here, we used the hESC-TNNT2-GFP line to create the human hESC-triple reporter (3R) line (described in more detail in the Reporter Cell Line Creation section below). To fluorescently visualize mesoderm, endoderm, and ectoderm germ layers, we used the human hESC-RUES2 GLR line generously provided by the Brivanlou Lab (Martyn et al., 2018)). Finally, we used the human 113 WT hiPSC line (113, Stanford CVI Biobank) to validate our hESC cVO results in a hiPSC line.

Construction of Lentiviral Plasmids. The two final lentiviral vector plasmids pLV-CDH5Pr-mOrange-SVPr-Zeo and pLV-TAGLNPr-CFP-SVPr-Bsd were constructed as described below. To facilitate selection and identification of cell lines transduced with lentivirus created with these vectors, we designed a series of double-cassette lentiviral vectors that feature cell-specific fluorescent reporter cassettes and constitutively expressed drug-selection cassettes. FIG. 10A-D illustrates the overall design of these vectors. First, a blank lentivirus vector pLV-B was derived from pSicoR (11579, Addgene). The plasmid pSicoR was digested by Apal-EcoRI, and re-ligated with an oligo adaptor (Adaptor-A/B) containing restriction sites XhoI, NotI, XbaI, and XmaI. The oligo adaptor and PCR primers used in this study are listed in Table 3.

Next, a cassette with the SV40 promoter (SVPr) controlling the expression of Zeocin (Zeo) was introduced into the blank lentivirus vector pLV-B to create the plasmid pLV-SVPr-Zeo. The template for the SVPr comes from the pGL3 luciferase reporter plasmid (E1741, Promega), and Zeo from the pGreenZeo human Nanog reporter plasmid (SR10030VA-1, System Biosciences). The cassette SVPr-Zeo was assembled by PCR with primer pairs SVPr-F/R and Zeo-F/R as previously described (An et al., 2010). The derived fragment was restricted with Xbal-Xmal and ligated into pLV-B at the same sites to create pLV-SVPr-Zeo. Then, the Blasticidin (Bsd) coding sequence (CDS) was introduced to replace the Zeocin CDS in pLV-SVPr-Zeo. The Bsd CDS was amplified from the pCMV/Bsd (BsdCassette™ Vector) plasmid (V51020, Thermo Fisher) with primer pair Bsd-F/R. The amplified fragment was digested with AgeI-XmaI and ligated into pLV-SVPr-Zeo at the same sites. Thus, the plasmid pLV-SVPr-Bsd was created.

The plasmid pLV-CDH5Pr-mOrange-SVPr-Zeo was constructed as follows. The cassette carrying the CDH5 (VE-Cadherin) promoter (CDH5Pr) controlling expression of the mOrange fluorescent protein was amplified from the plasmid pLV-CDH5Pr-mOrange (James et al., 2011), which is a gift from the Rafii Lab, Cornell, with primer pair VmO-F/R. The amplified fragment was restricted with XhoI-XbaI and ligated into pLV-SVPr-Zeo at the same sites to create pLV-CDH5Pr-mOrange-SVPr-Zeo.

The plasmid pLV-TAGLNPr-CFP-SVPr-Bsd was constructed by inserting a human transgelin (TAGLN) (SM22-alpha) promoter-directed CFP cassette (TAGLNPr-CFP) into pLV-SVPr-Bsd. This TAGLNPr-CFP cassette was obtained by PCR assembly of the TAGLNPr and the CFP CDS with primer pairs TGN-F/R and CFP-F/R as previously described (An et al., 2010). The TAGLN promoter sequence comes from the pEZX-TAGLN-GLuc plasmid (HPRM24364-PG02GeneCopoeia), and the CFP CDS from the pTagCFP-N plasmid (FP112, Evrogen). The derived fragment was restricted with Xhol-Xbal and ligated into pLV-SVPr-Bsd at the same sites to create pLV-TAGLNPr-CFP-SVPr-Bsd. Verification of plasmid construction was done by Sanger sequencing.

Lentivirus Production. To package plasmid in lentivirus, HEK 293 FT cells (R70007, Thermo Fisher) were transfected separately with target plasmids (pLV-CDH5Pr-mOrange-SVPr-Zeo or pLV-TAGLNPr-CFP-SVPr-Bsd, and the packaging plasmids psPAX2 (containing GAG and POL) (12260, Addgene) and pMD2.G (containing VSV-G) (12259, Addgene)) using Lipofectamine 2000 (11668019, Thermo Fisher). Transfected HEK293FT cells were incubated for 3 days and media collected daily. Media was then centrifuged at 3000 g for 15 min to remove cells and cell debris. The supernatant was concentrated using PEG-it Virus Precipitation Solution (LV810A, System Biosciences) according to the manufacturer's protocol.

Reporter Cell Line Creation. The human H9 hESC-TNNT2-GFP cell line (Wrighton et al., 2014) (H9-hTnnT2-pGZ-TD2, WiCell) was seeded on 6-well plates and first transduced with the lentivirus expressing CDH5Pr-mOrange-SVPr-Zeo. Cells were selected by Zeocin (0.5-3 μg/mL) treatment for 1-2 weeks. Genomic DNA was isolated from several clones, PCR was performed, and gel electrophoresis was used to verify expression of CDH5 (VE-Cadherin), mOrange, and Zeocin. This double reporter cell line was then transduced with the lentivirus expressing TAGLNPr-CFP-SVPr-Bsd to create the human H9 hESC-triple reporter (3R) cell line (hESC-3R). Cells were selected by Blasticidin (3 μg/mL) treatment for 1-2 weeks. Again, genomic DNA was isolated from several clones, PCR was performed, and gel electrophoresis was used to verify the presence of TAGLN (SM22-alpha), CFP, and Blasticidin (FIG. 10E-G). PCR primers are listed in Table 3.

Non-quantitative PCR. Non-quantitative PCR was performed using custom-designed PCR primers (Table 3). Genomic DNA was isolated with the PureLink Genomic DNA Mini Kit (K182000, Thermo Fisher) and quantified using a Nanodrop 2000 Spectrophotometer (ND-2000, Thermo Fisher) per the manufacturer's instructions. For PCR amplification, AccuPrime SuperMix I (12342, Thermo Fisher), custom primers for pluripotency and lentiviral markers (Table 3), and genomic DNA were combined. Non-template control (NT) reactions were prepared by substituting DNA with distilled water. Samples were transferred to a thermal cycler and the following cycling program was used: a) initial denaturation at 94° C. for 2 min; b) 30 cycles of 94° C., 30 sec; 60° C., 30 sec; 68° C., 1 min; c) final extension at 68° C. for 5 min. Reactions were maintained at 4° C. after cycling and then stored at-20° C. The PCR products and a 10-1500 bp DNA QuantLadder (50475, Lonza) were loaded in a 1.2% 16+1 double-tier FlashGel DNA Cassette (57029, Lonza), run, and visualized with the FlashGel System (57067, Lonza).

Human Pluripotent Stem Cell (hPSC) Culture. Human pluripotent stem cells (hPSCs) (both hESCs and hiPSCs) were maintained in the pluripotent state through daily feeding with Essential 8 (E8) medium (A1517001, Thermo Fisher Scientific). The medium was changed daily, and cells were passaged every 3-4 days with 0.5 mM EDTA (15575-020, Thermo Fisher Scientific) between a 1:6 and 1:12 split ratio. hPSCs were passaged on to tissue culture plates coated with Corning Matrigel membrane matrix (354234, Thermo Fisher Scientific) at a dilution of 1:100 for either maintenance or differentiation. To aid in passaging undifferentiated hPSCs by minimizing anoikis (dissociation-induced apoptosis), we cultured cells for 24 hours with 10 uM of the ROCK inhibitor Y-27632 (HY-10583, MedChem Express). Excess cells were frozen in 90% Knockout Serum Replacement (KOSR) (#10828010, Thermo Fisher Scientific) with 10% DMSO (#D2438, Sigma-Aldrich) in cryovials and frozen at −80° C. overnight and then subsequently transferred to liquid nitrogen storage.

Pluripotency Markers. Using standard protocols, undifferentiated hPSCs were labeled with primary antibodies for the pluripotency markers Oct-3/4 (mouse anti-human Oct-3/4 mAb (Clone 40), 1:400, 611202, BD Biosciences), Nanog (goat anti-human Nanog pAb, 1:100, AF1997, R&D Systems), and Sox2 (rabbit anti-human Sox2 mAb (Clone D6D9), 1:200, 3579S, Cell Signaling Technology). Secondary antibodies used were donkey anti-mouse IgG Alexa 488 for Oct-3/4, 1:500 (A21202, Thermo Fisher Scientific); donkey anti-goat IgG Alexa 555 for Nanog, 1:500 (A21432, Thermo Fisher Scientific); and donkey anti-rabbit IgG Alexa 647 for Sox2, 1:500 (A31573, Thermo Fisher Scientific). Cell nuclei were counterstained with 4′,6-diamidino-2-phenylindole (DAPI) (#D8417, Sigma-Aldrich, 1 mg/mL+0.1% Triton X-100 in PBS) or DRAQ5™ Fluorescent Probe Solution (5 mM) (62251, Thermo Fisher Scientific).

Stencil Creation for hPSC Colony Micropatterning. Our previous micropatterning method used a high-cost laser cutter that is not available in many labs. Here, to create stencils for hPSC colony micropatterning, we used a low-cost, widely available, table-top Silhouette Cameo cutting tool (B007R83VKE, Silhouette America, Amazon) to design and then cut circular patterns from a press-to-seal silicone sheet (P18178, Thermo Fisher Scientific). To fit in one well of a standard Falcon 48-well plate (353078, Thermo Fisher Scientific), the outer diameter of the circular patterns was cut to approximately 9.5 mm. To fit in one well of a standard Falcon 6-well plate (353046, Thermo Fisher Scientific), the outer diameter of the circular patterns was cut to approximately 34 mm. At the center of the 48-well pattern, a single 2 mm hole was cut to create a complete single-hole stencil; for the 6-well circular pattern, an evenly spaced array (3×3 to 7×7 with a pitch of 2 to 5 mm from center to center) of 2 mm holes were cut to create a complete multi-hole stencil array. The stencils were then washed 3 times with deionized (DI) water for 20 minutes. For previously used stencils, cleaning was performed 3 times for 30 minutes with Alconox anionic detergent (ALC1104, Midland Scientific) in an ultrasonic cleaning bath (Branson Ultrasonic Bath, Emerson Electric) to remove any residual cellular debris. After detergent cleaning, stencils were washed again 6 times with DI water for 15 minutes in the ultrasonic bath. Next, to render the silicone stencil surfaces hydrophilic, a handheld laboratory corona treater (12051A, Electro-Technic Products) was swept back and forth across the stencils approximately 2 times for 5 minutes. The stencils were then autoclaved at 15 psi at 121° C. for 30 minutes. Finally, the stencils were kept submerged in 100% ethanol until they were ready to be placed in the multi-well plates.

Single and Multiple hPSC Colony Micropatterning. Stencils for either a 48-well plate or a 6-well plate were placed in each well and pressed onto the well surface with sterile forceps. Next, ice cold Corning Matrigel membrane matrix (354234, Thermo Fisher Scientific) at a dilution of 1:100 in 0.25 mL ice cold RPMI-1640 (11875-093, Thermo Fisher Scientific) was poured into each well containing a stencil and was gelled by incubating at 37° C. for at least 1 hour. Undifferentiated hPSCs were dissociated with 0.5 mM EDTA (15575-020 Gibco/Thermo Fisher Scientific) and passaged as a single cell suspension that included 10 μM of the ROCK inhibitor Y-27632 (HY-10583, MedChem Express) to prevent dissociation-induced apoptosis. The cells at a concentration of 1.0-1.5×10⁶ cells/mL were introduced into the Matrigel-and stencil-containing wells and were allowed to attach at 37° C. overnight in 0.25 mL Essential 8 (E8) medium. The next day, stencils were removed with sterile forceps, leaving micropatterned cells attached on the 2 mm circular islands created by the 2 mm holes of the stencils. At this concentration, the 2 mm colonies were essentially 100% confluent in 2-3 days, showing good border integrity and uniform cell density. When colonies were filled in, the cells were fed with ice cold E8 which contained Matrigel at a dilution of 1:100 and immediately placed back in the incubator to minimize cold shock. This was required to allow the colonies to migrate beyond their 2 mm initial boundaries upon differentiation. The differentiation protocol was initiated when the colonies were essentially 100% confluent, typically 2-3 days after seeding. We observed successful differentiation on circular micropatterns at diameters of 2, 4, and 6 mm, but chose 2 mm because this size gave more repeatable results and allowed greater outward migration within the 48-well plates.

Growth Factors and Small Molecules. To induce mesoderm and subsequent cardiomyocyte differentiation, we used 4, 5, or 6 μM CHIR-99021 (CHIR) (S2924, Selleck Chemicals) and 5 uM IWR-1 (IWR) (S7086, Selleck Chemicals) and 5 ng/ml FGF-2 (FGF2) (100-18B, PeproTech). These two small molecules and one growth factor served as the basis for our baseline (Control group) differentiation method.

To simultaneously induce endothelial cell vasculogenesis and angiogenesis along with cardiomyocyte differentiation, we added 50 ng/ml VEGF (VEGF) (100-20, PeproTech); 5 ng/ml FGF-2 (FGF2) (100-18B, PeproTech); 10 μM SB431542 (SB) (S1067, Selleck Chemicals); 50 ng/ml Angiopoietin-2 (ANG2) (130-07, PeproTech); 50 ng/ml Angiopoietin-1 (ANG1) (130-06, PeproTech); and select components of the VascuLife® VEGF LifeFactors® Kit (LS-1020, Lifeline Cell Technology) (5 ng/ml EGF, 15 ng/mL IGF-1, 50 μg/mL ascorbic acid, 0.75 U/mL heparin sulfate, 1 ug/mL hydrocortisone, 5 ng/mL VEGF (not added), 5 ng/ml FGF-2 (not added), 2% FBS (not added), 30 mg/mL/15 ug/mL gentamicin/amphotericin B (not added), 10 mM glutamine (not added)).

To simultaneously induce smooth muscle cell differentiation with cardiomyocyte and endothelial cell differentiation (described in more detail in the Cardiovascular Differentiation section), we added 2.5 or 10 ng/ml PDGF-BB (PDGFBB) (100-14B, PeproTech) and 0.5 or 2 ng/ML TGF-β1 (TGFβ) (100-21C, PeproTech).

Finally, to maintain isolated endothelial cells, we used the EGM-2 Bullet Kit (CC-3162, Lonza) (EGF, IGF-1, ascorbic acid, heparin sulfate, hydrocortisone, VEGF, FGF-2, FBS, and gentamicin/amphotericin-B).

To induce mesendoderm and subsequent hepatocyte differentiation, we used 100 ng/ml Activin-A (338-AC, R & D Systems); 10 ng/ml BMP-4 (314-BP, R & D Systems); 3 μM CHIR-99021 (S2924, Selleck Chemicals); 10 μM LY294002 (PI3K-AKT inhibitor) (70920, Cayman Chemical Company); 50 ng/ml FGF-10 (100-26, PeproTech); 50 ng/ml HGF (100-39H, PeproTech); 50 ng/ml Oncostatin-M (300-10, PeproTech); 10 μM Dexamethasone (1126/100, Tocris); and HCM Hepatocyte Culture Medium Bullet Kit (CC-3198, Lonza).

Baseline Cardiomyocyte (CM) Differentiation. For baseline cardiomyocyte (CM) differentiation (Control group), hPSCs were transferred to Matrigel-coated non-micropatterned and micropatterned surfaces as described above and differentiated in RPMI-1640 media (61870, Thermo Fisher Scientific) supplemented with B27 without insulin (A1895601, Gibco/Thermo Fisher Scientific). Our baseline differentiation method was based on a small molecule-based monolayer method (Lian et al., 2012; Burridge et al., 2014). Briefly, on the first day (Day 0) of differentiation, basal medium was supplemented with 4, 5, or 6 μM CHIR99021 (CHIR) (S2924, Selleck Chemicals) and 5 ng/mL FGF-2 (FGF2) (100-18B, PeproTech). On Day 2, the medium was replaced with basal medium containing B27 without insulin. On Day 3, the medium was replaced with basal medium containing B27 without insulin and containing 5 uM IWR-1 (IWR) (S7086, Selleck Chemicals). On Day 5 the medium was replaced with basal medium containing B27 without insulin. From Days 7-16, the basal medium was changed to contain B27 with insulin (17504044, Thermo Fisher Scientific) and was replaced every 48 hours thereafter. Cardiomyocytes generally began spontaneously beating sometime between Days 7 and 10. hPSC-CMs were collected on Day 16 for further downstream studies. More complex cardiac vascularized organoid (cVO) differentiation described below was based on this baseline differentiation and contained several additional small molecules and growth factors to promote simultaneous cardiac and vascular co-differentiation.

Cardiac Vascularized Organoid (cVO) Differentiation. See FIG. 3C, FIG. 10H-I, and Table 1 for an overview of our thirty-four (34) cardiovascular (CV) differentiation screening conditions to produce cVOs. The Control groups utilized only CHIR, FGF2, and IWR. To begin, hPSCs suspended in single cells were transferred to Matrigel-coated non-micropatterned or micropatterned surfaces as described above and differentiated in RPMI-1640 media (61870, Thermo Fisher Scientific) supplemented with B27 without insulin (A1895601, Thermo Fisher Scientific) (RB-I). The CV differentiation was based on the baseline differentiation described previously to promote CM differentiation and contained several additional small molecules and growth factors to promote simultaneous endothelial and smooth muscle cell co-differentiation. On Day 0, for primarily mesoderm induction, basal medium was supplemented with 4 or 5 μM CHIR99021 (CHIR) (S2924, Selleck Chemicals) and 5 ng/ml FGF-2 (FGF2) (100-18B, PeproTech). On Day 3, for CM induction, 5 uM IWR-1 (IWR) (S7086, Selleck Chemicals) was added.

On Days 5-16, to simultaneously induce endothelial cell vasculogenesis and angiogenesis along with the cardiomyocyte differentiation, we added combinations of 50 ng/ml VEGF-165 (VEGF) (Days 5, 7, 9, 11, 13) (100-20, PeproTech); 5 ng/ml FGF-2 (FGF2) (Days 7, 9, 11, 13) (100-18B, PeproTech); 10 uM SB431542 (SB) (Days 7, 9, or 11) (S1067, Selleck Chemicals); 50 ng/ml Angiopoietin-2 (ANG2) (Days 5 and 7) (130-07, PeproTech); 50 ng/ml Angiopoietin-1 (ANG1) (Days 9 and 11) (130-06, PeproTech); and select components of the VascuLife® VEGF LifeFactors® Kit (Aghajanian et al., 2016) (LS-1020, Lifeline Cell Technology) (Days 5, 7, 9, 11, and 13) (5 ng/ml EGF, 15 ng/ml IGF-1, 50 μg/mL ascorbic acid, 0.75 U/mL heparin sulfate, 1 g/mL hydrocortisone).

On Days 7-16, to simultaneously induce smooth muscle cell differentiation with cardiomyocyte and endothelial cell differentiation, we added 2.5 or 10 ng/ml PDGF-BB (PDGFBB) (Days 7, 9, 11, 13) (100-14B, PeproTech) and 0.5 or 2 ng/ml TGF-β1 (TGFβ) (Days 7, 9, 11, 13) (100-21C, PeproTech).

From Days 5-16, unless otherwise noted, the basal medium was changed to contain B27 with insulin (RB+I) (17504044, Thermo Fisher Scientific) and was replaced every 48 hours thereafter. Cardiomyocytes within Control and cVO groups generally began spontaneously beating sometime between Days 7 and 10. Cells were collected at various time points between Days 0 and 16 for further downstream studies.

For 3D cVO creation, two methods were used. In the first method, stencils with single 2 mm center holes were placed in 24-well plates with Softwell Easy Coat hydrogels with a custom stiffness of 16 kPa (SW24-EC-C, Matrigen) and hESC-3R cells were micropatterned as described above. In the second method, to increase throughput and eliminate using stencils, each well of a black 96-well plate (M0562-32EA, Sigma-Aldrich) was freshly coated with a central, 2 uL drop of Matrigel (1:400 dilution) and was then seeded with 125,000 hESC-3R cells. Cells were allowed to attach and grow for 2-4 days forming a single, homogenous, round colony in the center of each well. The same differentiation protocol for 2D cVOs was then used to create 3D cVOs from days 0-14, using a range of CHIR from 6.0-8.5 μM.

Hepatic Vascularized Organoid (hVO) Differentiation. See FIG. 7A-C and Table 2 for an overview of our hepatovascular (HV) differentiation to produce hepatic vascularized organoids (hVOs). We tested 3 differentiation conditions as follows: i) Control (day 20 baseline hepatic differentiation, with no vascularization factors added); ii) hVO-D3 (day 20 hVOs created by adding vascularization factors at day 3 of differentiation); and iii) hVO-D6 (day 20 hVOs created by adding vascularization factors at day 6 of differentiation). To begin, hPSCs suspended in single cells were transferred to Matrigel-coated micropatterned surfaces as described above and differentiated in DMEM/F12 with HEPES media (11330032, Thermo Fisher Scientific) supplemented with 1× GlutaMax (35050061, Thermo Fisher Scientific), 0.5 mg/500 mL media of Poly (vinyl) alcohol (PVA) (P8136, Sigma-Aldrich), 1× Insulin-Transferrin-Selenium (ITS) (41400045, Thermo Fisher Scientific), 1× chemically defined lipid concentrate (11905031, Thermo Fisher Scientific), 20 uL/500 mL media of 1-thioglycerol (M6145, Sigma-Aldrich), 1× non-essential amino acids (NEAA) (11140050, Thermo Fisher Scientific), and 1 mM sodium pyruvate (11360070, Thermo Fisher Scientific). The hVO differentiation contained concentrations of the small molecules and growth factors found to promote simultaneous endothelial and smooth muscle cell production from the results of our cVO differentiation method above. On Day 0, for mesendodermal induction, basal medium was supplemented with 100 ng/mL Activin-A (ActA) (338-AC, R & D Systems), 10 ng/ml BMP-4 (BMP4) (314-BP, R & D Systems), 3 uM CHIR99021 (CHIR) (S2924, Selleck Chemicals), 100 ng/ml FGF-2 (FGF2) (100-18B, PeproTech) and 10 uM LY294002 (LY) (70920, Cayman Chemical Company). On Day 3, for foregut induction, 50 ng/mL FGF-10 (FGF10) (100-26, PeproTech) was used. On Day 6, for hepatoblast induction, 10 ng/ml FGF10 and 10 ng/ml BMP4 was used. Finally, to induce hepatocytes, 50 ng/ml Hepatocyte Growth Factor (HGF) (100-39H, PeproTech), 50 ng/ml Oncostatin-M (OncoM) (300-10, PeproTech), and 10 uM Dexamethasone (DEX) (1126, Tocris) was used.

Starting on Days 3 or 6 and continuing until Day 20, to simultaneously induce endothelial cell vasculogenesis and angiogenesis along with the hepatocyte differentiation, we added combinations of 50 ng/ml VEGF-165 (VEGF) (Days 3 or 6, 9, 11, 13, 15, 17, and 19) (100-20, PeproTech); 5 ng/ml FGF-2 (FGF2) (Days 6 or 9, 11, 13, 15, 17, and 19) (100-18B, PeproTech); 10 uM SB431542 (SB) (Days 6 or 9, 11, 13, 15, 17, and 19) (S1067, Selleck Chemicals); 50 ng/ml Angiopoietin-2 (ANG2) (Days 3 or 6, and 9) (130-07, PeproTech); 50 ng/ml Angiopoietin-1 (ANG1) (Days 11 and 13) (130-06, PeproTech); and select components of the VascuLife® VEGF LifeFactors® Kit (LS-1020, Lifeline Cell Technology) (Days 3 or 6, 9, 11, 13, 15, 17, and 19) (5 ng/ml EGF, 15 ng/ml IGF-1, 50 μg/mL ascorbic acid, 0.75 U/mL heparin sulfate, 1 μg/mL hydrocortisone).

On Days 6-20, to simultaneously induce smooth muscle cell differentiation with hepatocyte and endothelial cell differentiation, we added 2.5 ng/ml PDGF-BB (PDGFBB) (Days 6, 9, 11, 13, 15, 17, and 19) (100-14B, PeproTech) and 0.5 ng/ml TGF-β1 (TGFβ) (Days 15, 17, and 19) (100-21C, PeproTech).

From Days 3-9, the basal medium was changed to contain B27 with insulin (17504044, Thermo Fisher Scientific) (RB+I) and was replaced every 48 hours. From Days 9-20, the basal medium was changed to HCM medium (CC-3198, Lonza) and was replaced every 48 hours. Cells were collected on Day 20 for endpoint analyses.

Time-lapse Microscopy. Initial time-lapse microscopy was performed with the Incucyte S3 Live-Cell Analysis Instrument (Essen Bioscience). Twenty-four (24)-well plates containing a single 2, 4, or 6 mm circular micropattern of undifferentiated hESC-TNNT2-GFP cells (H9-hTnnT2-pGZ-TD2, WiCell) (Wrighton et al., 2014) in the center of each well were subjected to the baseline cardiomyocyte differentiation condition and imaged at ten (10) timepoints from Days 0-9 (DO-D9). Phase contrast and GFP signals were obtained with a 4× objective in a square mosaic containing the entire surface area of each well. At all time points, plates were kept at 37° C. and 5% CO2.

To increase throughput, screening time-lapse microscopy was performed with the Cytation 5 Cell Imaging Multi-Mode Reader (BioTek). Five (5) forty-eight (48)-well plates (204 out of 240 wells) were imaged over the differentiation time-period. The plates contained a single 2 mm circular micropattern of undifferentiated hESC-3R cells in the center of each well subjected to thirty-four (34) differentiation conditions, with nominally 4-6 replicates per condition, imaged at six (6) timepoints (Days 3, 5, 8, 10, 12, and 16 (D3-D16)).

Phase contrast, CFP, GFP, and mOrange signals in four (4) independent channels were obtained and the following excitation light emitting diodes (LEDs), excitation (Ex) filters, emission (Em) filters, and dichroic (Di) mirrors were used for the fluorescence channels: CFP: LED-465nm, Ex-445/45 nm, Em-510/42 nm, Di-482 nm; GFP: LED-465 nm, Ex-469/35 nm, Em-525/39 nm, Di-497 nm; mOrange-LED: 523 nm, Ex-531/40 nm, Em-593/40 nm, Di-568 nm. Light intensity and exposure time were adjusted for each channel to minimize under-or overexposure. A 4× objective (NA 0.13) was used to acquire a 4×4 array in a single focal plane for all channels, resulting in a rectangular mosaic containing the circular micropattern of cells and an area surrounding each micropattern which allowed imaging of migrating cells off each micropattern over time. At all time points, plates were kept at 37° C. and 5% CO2 while imaging.

When using 48-well plates to screen 34 conditions and 6 replicates per condition, the number of wells needed was 204. Therefore, the theoretical number of acquired raw images was 78,336 images (16 images/channel/mosaic×4 channels/mosaic×6 days/well×204 wells/day).

The actual number of acquired raw images was 75,648, corresponding to 197 wells (out of 204 wells), with 7 wells excluded because of debris, detachment of micropatterns, and incomplete micropatterns at the start of differentiation (representing 3.4% exclusion of wells).

After all raw images were acquired, image analysis was performed with Gen5 v3.05 software (BioTek). Images in each channel and each 4×4 array were stitched to create a single mosaic image; 4728 mosaics were created, and each mosaic image was downsized by 25%. Next, image processing of each mosaic was performed to reduce background independently in each channel. Finally, for each channel, thresholding was performed on fluorescence intensity along with minimum and maximum object size to calculate the total Phase, CFP, GFP, and mOrange total fluorescence area in each well for each condition at each timepoint. Each mosaic image was computationally masked by a circular “plug” to eliminate fluorescence artifact created by well edges. For each condition, values for replicate samples were summed, and the mean and standard deviation were calculated.

Widefield Microscopy. Either an AxioObserver Z1 (Zeiss) or a Keyence BZ-X700 (Keyence) inverted microscope was used to visualize undifferentiated hPSCs and their derivatives. The Zeiss microscope was equipped with 2.5×, 4×, 10×, 20×, 40×, and 63× objectives, a Lambda DG-4 300 W Xenon light source (Sutter Instruments), an ORCA-ER CCD camera (Hamamatsu) to visualize Phase, CFP, GFP, and mOrange channels, and ZEN (Blue Edition) v2.6 software (Zeiss) for image stitching, z-stack image capturing, and incubation control. The Keyence microscope was equipped with 2×, 4×, 10×, and 20× objectives, an 80 W metal halide lamp, a monochrome CCD camera to visualize Phase, CFP, GFP, and mOrange channels, and BZ-X Analyzer software for optical sectioning, image stitching, z-stack image capturing, and incubation control. Both microscope incubation systems were maintained at 37° C. and 5% CO2.

Confocal Microscopy. Either a Zeiss LSM710 Inverted Confocal Microscope (Carl Zeiss) (Stanford Neuroscience Microscopy Service Core Facility) or a Leica SP8 White Light Confocal (Leica) (Stanford Cell Sciences Imaging Core Facility) confocal microscope was used to visualize live and fixed undifferentiated hPSCs, individual hPSC-derived cells, and cVOs. The Zeiss LSM710 confocal (AxioObserver Z1 inverted microscope base) is equipped with the following: environmental chamber to maintain samples at 37° C. and 5% CO2; motorized X-Y stage and Z focus; 10× Plan Apochromat (NA 0.45); 20× Plan Apochromat (NA 0.45); 40× Plan Neofluar (oil) (NA 1.30); 63× Plan Apochromat (oil) (NA 1.40); 405 nm diode laser (30 mW); 458/488/514 nm argon laser (35 mW); 561 nm diode-pumped solid-state (DPSS) laser (20 mW); 633 nm helium-neon (HeNe) laser (5 mW); temperature-stabilized VIS-acousto-optical tunable filter (AOTF) for simultaneous intensity control; and Zeiss ZEN Black v2.6 software. The Leica SP8 confocal (DMI 6000 inverted microscope platform) is equipped with the following: environmental chamber to maintain samples at 37° C. and 5% CO2; motorized X-Y stage and galvanometer Z focus; 10× Plan Apochromat (NA 0.40); 20× Plan Apochromat (oil) (NA 0.75); 40× Plan Apochromat (oil) (NA 1.30); 63× Plan Apochromat (oil) (NA 1.40); 405 nm laser (50 mW); super continuum, white light (WLL) pulsed laser (avg. power 1.5 mW), 470 nm to 670 nm, 78 MHz; acoustical optical beam splitter (AOBS) for selection of up to 8 discrete laser lines (1 nm precision); 3 hybrid-GaAsP detectors; 2 standard fluorescent photomultiplier tubes (PMT); and Leica LAS AF software. Offline image analysis was also performed with Imaris v9.6.1 and ImageJ (Fiji) v2.1.0 software.

Immunocytochemistry (ICC). Primary and secondary antibodies were reconstituted in sterile PBS per manufacturer guidelines. Samples were fixed in IC Fixation Buffer (FB001,Thermo Fisher Scientific, diluted in PBS) or 4% paraformaldehyde (15713S, Electron Microscopy Sciences) for 10-15 minutes and washed with PBS for 10 minutes three times. To begin staining, samples were permeabilized with 0.1% Triton-X for 30 minutes at room temperature and then incubated in blocking buffer (10% normal goat serum and 0.05% Tween in PBS) for 1 hour at room temperature. Then the primary antibody (prepared in blocking buffer) was applied for 4° C. overnight. Washing buffer (0.05% Tween in PBS) was applied to the samples for 5 minutes at room temperature three times. Secondary antibody (typically at 1:500 dilution) was applied for 1 hour at room temperature and followed by three 5 minutes washes in wash buffer. After a final 5-minute rinse with sterile PBS, samples were counterstained with either 4′,6-diamidino-2-phenylindole (DAPI) (D8417, Sigma) or DRAQ5™M Fluorescent Probe Solution (62251, Thermo Fisher Scientific). Samples were either analyzed immediately or stored at 4° C. in PBS.

Bulk RNA-Sequencing (RNA-seq). hESC-3R (1 group, day 0, n=3, 3 total samples; note, these samples were used for both cardiac and hepatic analyses), Control, and cVO samples (2 groups, days 2, 5, 8, 10, 12, and 16, n=3 per sample, 36 total samples) were prepared with the Direct-zol RNA Microprep w/Zymo-Spin IC Columns (R2060, Zymo Research) and sequenced. For hVO analysis, Control, hVO-D3, and hVO-D6 samples (3 groups, day 20, n=3 per sample, 9 total samples) were prepared in the same fashion. Each replicate had an average of 24.4 million 150 bp long paired-end reads. FastQC v0.11.2 was used for sequencing quality assessment. Reads were then aligned to the GRCh38 (hg38) reference genome using STAR v2.5.3a with splice junctions being defined in GTF file (obtained from GRCh38). An average of 85.3% of reads was aligned to the reference transcriptome. Expression at the gene level was determined by read counts using RSEM 1.2.30. Differently expressed genes (DEGs) with fold-change were further detected by DEseq2 version 1.10.1 for comparable conditions. Based on the DEG data set, the Euclidean distances between samples, as calculated from the rlog transformation, were calculated and plotted as a heatmap.

To determine heatmap scale, the following was performed. After hierarchical clustering using Median Clustering or the Weighted Pair Group Method with Median Clustering (WPGMC) method together with the Spearman correlation (square of Euclidean distance) method for distance measurement on the log-transformed gene expression table, the values were further scaled in the row direction. This centers and standardizes each row separately to row Z-score.

Principal component analysis (PCA) was performed to extract the main information from the DESeq2 transformed (rlog) data set so that each successive axis was ordered by decreasing order of variance. Approximately 60% of all transcripts (15994 of 26470 total genes) after minimal pre-filtering to keep only rows which contained at least 10 total read/raw counts were used for PCA. PCA plots were used to visualize the batch effects and overall effect of experimental covariates. Numbers on each plot axis indicate frequency of transcripts described by each principal component.

Weighted Gene Co-Expression Network Analysis (WGCNA) was used for hierarchical clustering with dynamic tree cut to identify gene co-expression modules; the modules were further analyzed using Ingenuity Pathway Analysis (IPA) (Qiagen), described below. RNA-seq data of hESC, Control, cVO, and hVO samples are deposited in the NIH Gene Expression Omnibus (GEO) under GSE185194.

Ingenuity Pathway Analysis (IPA). We used Ingenuity Pathway Analysis (IPA) (v65367011, Qiagen) of the temporal bulk-RNA-sequencing (RNA-seq) weighted gene co-expression network analysis (WGCNA) of Control and cVO samples to show vascularization and cardiogenesis regulator effect networks. In addition, we used IPA to show predicted top canonical pathways, upstream regulators, functions, regulator effect networks, and networks of Day 16 cVOs compared to D16 Control samples. WGCNA Module 16 (Pale Turquoise) within the “Vascularization Genes” cluster (FIG. 4L) contained 1258 analysis-ready genes with a Log2FoldChange ranging from −0.9 to 15.1 (13 down regulated and 1245 upregulated genes) and revealed regulator effect networks related to vascularization. WGCNA Module 12 (Dark Grey) within the “Cardiomyocyte/Endothelial/Smooth Muscle/Fibroblast Genes” cluster (FIG. 4L) contained 3719 analysis-ready genes with a Log2FoldChange ranging from −1.2 to 17.8 (46 down regulated and 3673 upregulated genes) and revealed regulator effect networks related to cardiogenesis. For analysis of Day 16 cVOs compared to D16 Control samples, 15,671 differentially expressed genes (Log2FoldChange ranging from −7.2 to 8.3) were reduced to 1001 analysis-ready genes by analyzing only differentially expressed genes with Log2FoldChange cutoff values of below −2 and above +2 and padj value equal to or greater than 0.05.

Single-Cell RNA-Sequencing (scRNA-seq). Control and cVO samples (day 16, n=6 pooled organoids per sample) were dissociated using the Pierce™ Primary Cardiomyocyte Isolation Kit (88281, Thermo Fisher Scientific) according to the manufacturer's instructions. A 10 uL sample of dissociated single cells was visualized with a fluorescent microscope (Zeiss) to verify the presence of GFP+, mOrange+, and CFP+ cells in the sample. Next, single cells (with an end-target of ˜4000 cells per sample) were used to create Gel Bead-In EMulsions (GEMs) by using the Chromium Single Cell 3′ Library & Gel Bead Kit v2 kit (PN-120267, 10X Genomics). Libraries were generated according to the manufacturer's instructions and sequenced using the HiSeq2500 instrument (Illumina) with a read length of PE150 base pairs at each end and ˜50 M reads per sample.

Sequences were demultiplexed into FASTQ files using Cell Ranger 3.0.0 (10x Genomics) and then aligned to the GRCh38 (hg38) reference genome. Count matrices were generated using the count function with default settings. Results were initially visualized with Loupe Cell Browser v3.0.1 (10X Genomics) and then detailed downstream analysis was performed with the Seurat package (Stuart et al., 2019) (v.3.1.5 and v.4.0.0) in RStudio (v1.4.1106, RStudio) using R (v3.6.1 and 4.0.4, R Core).

Barcodes, features (genes), and matrix files were loaded into RStudio as a Seurat object using CreateSeuratObject with a minimum of 5 cells and 200 genes, and percent mitochondrial genes were identified. Cells with less than 200 or greater than 5000 unique genes, or mitochondrial content over 10% were discarded. Using NormalizeData, the data was then log-normalized and scaled with a factor of 10,000. FindVariableFeatures was then used to identify the top 2,000 highly variable genes using vst as the selection method. ScaleData was then used to apply a linear transformation. Next RunPCA was used to perform linear dimensional reduction by principal component analysis (PCA). JackStraw with 100 iterations was then used to identify statistically significant (P<0.001) principal components and ElbowPlot identified an ‘elbow’ at approximately 15 principal components (PCs). Clustering was performed using FindNeighbors and FindClusters using 15 PC dimensions and 0.2 resolution. Non-linear dimensional reduction was performed using RunUMAP and Uniform Manifold Approximation and Projection (UMAP) plots were visualized using DimPlot. Finally, differentially expressed genes were identified with FindAll Markers and violin, feature plots, and heat maps were visualized with VInPlot, FeaturePlot, and DoHeatmap. scRNA-seq data of Control and cVO samples are deposited in the NIH Gene Expression Omnibus (GEO) under GSE185194.

scRNA-seq Public Data. We compared our Control and cVO scRNA-seq data to a publicly available scRNA-seg dataset from a 6.5 post-conception week (PCW) human heart (Asp et al., 2019). The accession number for that raw sequencing dataset is European Genome-phenome Archive (EGA): EGAS00001003996. Count matrices were downloaded and analyzed initially with Seurat v3.1.5 and reanalyzed with v4.0.0.

Cell-Cell Communication Analysis. As we have previously described (Paik et al., 2018), to quantify the putative cell-cell communication in the cardiovascular cellulome, we obtained human ligand-receptor pairs compiled by Ramilowski et al. Briefly, we defined a ligand or receptor as “expressed” in a particular cell type if its expression is higher than 0 in more than 10% of cells in that cell type for the gene encoding the ligand or receptor. We linked any two cell types where the ligand was expressed in the former cell type and the receptor in the latter to define networks of cell-cell communication. The directionality in this relationship is visible where lines connecting cell populations are colored according to the population broadcasting the ligand and connect via an arrowhead to the population expressing the receptor, thus demonstrating a capability to receive the signal. In addition, line thickness correlates with the interaction number. To plot networks, we used the igraph and circlize R packages.

Nitric Oxide (NO) Testing. To determine the nitric oxide (NO) producing ability of hPSC-ECs in cVOs, we used the Nitric Oxide (total) detection kit (ADI-917-020, Enzo). The enzymatic conversion of nitrate to nitrite by nitrate reductase, followed by the Griess reaction to form a colored azo dye product, allowed assessment of NO produced by the hPSC-ECs. Quantification of NO produced was achieved by measuring absorption at 540-570 nm with the use of the Cytation 5 Cell Imaging Multi-Mode Reader (BioTek), averaged from four independent experiments.

Electrical Stimulation. For electrical stimulation studies, multi-well plates containing Control or cVO samples were placed in a customized upright optical imaging system (121517T1UP, SciMedia) and paced in Tyrode's solution (T2397, Sigma). Samples were then point stimulated with a stimulus generator (SIU-102, Warner Instruments). The electrical stimulation consisted of a biphasic waveform with peak-to-peak amplitude of 5-10 V, pulse width of 2 to 5 ms, and frequency of 1 or 2 Hz; point stimulation was delivered through a custom-made 1 mm two-wire platinum anode/cathode electrode.

Calcium Dye Imaging (CDI). For CDI, the Fluo-4 Direct Calcium Assay kit was used (F10471, Thermo Fisher) as per the manufacturer's instructions as previously described. Briefly, Fluo-4 loading solution was incubated with the samples at 37° C. for 30 min, washed twice with HBSS (14025092, Thermo Fisher), and resuspended Tyrode's solution. Fluorescence was measured at 495+/−20 nm excitation and 515+/−20 nm emission. Videos were taken with an Evolve 512 Delta EMCCD camera (Photometrics) using Micromanager v1.4 software (Vale Lab, UCSF) at 90 fps for 10 seconds of Control or cVO samples beating spontaneously or electrically stimulated at 1 or 2 Hz. In each video frame, regions of interest (ROIs) were analyzed for changes in dye intensity f/f0, with the resting fluorescence value f0 determined at the first frame of each video. Background intensity was subtracted from all values, and plots were normalized to zero. BV-Ana software (SciMedia) was used to quantify conduction velocity and beating frequency.

Contractility Imaging. We assessed the contractility of Control and cVO groups in a similar manner as we have previously described. Contraction of beating CMs within the micropatterns was recorded with high-resolution motion capture tracking (75 fps) using the SI8000 Live Cell Motion Imaging System (Sony Corporation). During data collection, cells were maintained under controlled humidified conditions at 37° C. with 5% CO2 and 95% air in a stage-top microscope incubator (Tokai Hit). Functional parameters were assessed from the averaged contraction-relaxation waveforms from 10 sec recordings, using the SI8000C Analyzer software (Sony Corporation). The software was used to detect motion vectors and quantify beating rate, contraction velocity, relaxation velocity, and contraction-relaxation peak interval of cVOs.

Signaling Pathway Inhibition. For NOTCH signaling pathway inhibition we used 0, 1, and 10 uM DAPT (GSI-IX) (S2215, Selleck Chemicals) added at day 0 of differentiation. For BMP signaling pathway inhibition, we used 0, 0.1, and 1 uM Dorsomorphin (P5499, Sigma-Aldrich) added at day 0 of differentiation. For VEGF signaling pathway inhibition we used 0, 0.1 and 1 ug/mL Angiostatin (SRP6032, Sigma-Aldrich) added at day 0 of differentiation.

Teratogen Drug Testing. For teratogen drug testing, we used 0, 8, and 80 ug/mL (±)-Thalidomide (T144, Sigma-Aldrich) added at day 0 of differentiation. Thalidomide is an inhibitor of the FGFR2, NFKB1, and TNF pathways. We also used 0 and 10 nM of fentanyl added at day 0 of differentiation.

Machine Learning. The general linear model and multiple regression fits were performed using scikit-learn. The general linear models had 100 coefficients that accounted for 9 growth factor/small molecule combinations and 1 bias term at 10 lags and was fit to the mean fluorescence area across 6 replicates in each condition. The multiple regression models had 7 coefficients accounting for the 7 measured days and was fit to all measured data points.

Statistical Analysis. Statistical analyses were performed using Prism 9 (GraphPad Software, LLC) and JMP Pro 15 (SAS Institute, Inc.) software. Data was first analyzed for normality and lognormality using the Shapiro-Wilk and D′Agostino & Pearson tests in Prism 9. If data comprised two normally distributed groups, a parametric unpaired two-tailed Student's t test was performed to determine significant differences. If data comprised two non-normally distributed groups, a nonparametric two-tailed Wilcoxon/Mann-Whitney U test was performed. If data comprised greater than two normally distributed groups, a parametric one-way or two-way ANOVA was performed. If data comprised greater than two non-normally distributed groups, a nonparametric Kruskal-Wallis test was performed. Subsequent multiple comparisons correction analysis was performed using the parametric Tukey's test for normally distributed data and the nonparametric Dunn's test for non-normally distributed data. Data are expressed as mean±standard deviation (SD). The p-values for significance differences are as follows: *p<0.05, **p<0.01, ***p<0.001, ****p<0.0001, not significant (ns). Statistical methods were not used to predetermine sample size. Samples were randomized with respect to applied differentiation conditions and assays. The investigators were not blinded to samples, experiments, and assessments of outcome. Detailed information regarding sample size, statistical analyses, and statistical significance is included in each figure.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method for in vitro differentiation of pluripotent stem cells to vascularized organoids comprising spatially organized and hierarchical branching of vessels, the method comprising:
initiating a culture with geometrically patterned pluripotent stem cells (PSC); and
step-wise differentiating the cells into germ layers, progenitor cell types, and differentiated cell types with defined agents acting on differentiation pathways.

2. The method of claim 1, wherein the geometric patterning of PSC comprises coating a specific region of from about 1 mm to about 10 mm on the surface of a culture vessel with a matrix, where the PSC are allowed to attach to the matrix at the initiation of culture.

3. The method of claim 2, wherein the coating step comprises adhering a stencil comprising a hole of from about 1 mm to about 10 mm, and pouring a matrix solution over the stencil.

4. The method of any of claims 1-3, wherein the PSC are human cells.

5. The method of any of claims 1-4, wherein the vascularized organoids are 2-dimentional or 3-dimensional.

6. The method of any of claims 1-5, wherein the vascularized organoid is a cardiac vascularized organoid, the method comprising simultaneous in vitro co-differentiation of atrial and ventricular cardiomyocytes, arterial and venous endothelial cells, smooth muscle cells, endocardial cells and epicardial cells.

7. The method of claim 6, comprising the steps of:
(a) contacting the micropatterned PSC with an effective dose of a WNT pathway activator and an effective dose of an activator of the FGF pathway agent for a period of from about 24 to about 72 hours to induce mesoderm;
(b) contacting the cells of step (a) with an effective dose of a Wnt inhibitor for a period of from about 24 to about 72 hours to induce cardiomyocytes;
(c) contacting the cells of step (b) with an effective dose of a VEGF agonist; an effective dose of an activator of the FGF pathway, an effective dose of an inhibitor of the SMAD pathway; an effective dose of an angiopoietin activating agent to simultaneously induce endothelial cell vasculogenesis and angiogenesis along with the cardiomyocyte differentiation for a period of from about 10 to 15 days; and
(d) contacting the cells of step (b) with an effective dose of a PDGF pathway activating agent; and a TGF-β1 activating agent to simultaneously induce smooth muscle cell differentiation with cardiomyocyte and endothelial cell differentiation for period of from about 10 to 15 days.

8. The method of claim 7, wherein the WNT pathway activator in step (a) is CHIR99021 at an effective dose of from about 1-20 μM; and the activator of the FGF pathway is FGF-2 at an effective dose of from about 1 ng/ml to about 20 ng/ml.

9. The method of claim 7 or claim 8, wherein the Wnt inhibitor in step (b) is IWR-1, where the effective dose is from about 0.1 μM to about 100 μM.

10. The method of any of claims 7-9, wherein in step (c) the VEGF agonist is VEGF-165 at an effective dose of from about 5 ng/ml to about 100 ng/ml; the activator of the FGF pathway is FGF-2 at an effective dose of from about 1 ng/ml to about 20 ng/ml; the inhibitor of the SMAD pathway is SB431542 at an effective dose of from about 1 μM to about 50 M; and the angiopoietin activating agent is Angiopoietin-2 (ANG2), or a combination of Angiopoietin-1 and -2 at an effective dose of from about 5 to about 100 ng/ml.

11. The method of any of claims 7-10, wherein medium of step (c) further comprises 5 ng/ml EGF, 15 ng/ml IGF-1, 50 g/mL ascorbic acid, 0.75 U/mL heparin sulfate, and 1 μg/mL hydrocortisone.

12. The method of any of claims 7-11, wherein in step (d) the PDGF pathway activating agent is PDGF-BB at an effective dose of from about 1 ng/ml to about 25 ng/ml; and the TGF-β1 activating agent is TGF-β1 at an effective concentration of from about 0.1 ng/ml to about 5 ng/ml.

13. The method of any of claims 1-5, wherein the vascularized organoid is a hepatic vascularized organoid, the method comprising simultaneous in vitro co-differentiation of hepatocytes, and a branching network of endothelial cells and smooth muscle cells.

14. The method of claim 13, comprising the steps of:
(a) contacting the micropatterned PSC with an effective dose of an activator of TGF-β pathway; an effective dose of an activator of BMP signaling pathway; an effective dose of a WNT pathway activating agent; an effective dose of a PI3K pathway inhibitor for a period of from about 24 to about 72 hours to induce mesendoderm;
(b) contacting the cells of step (a) in the presence of an effective concentration of an FGF activator to induce foregut;
(c) contacting the cells of step (b) with an effective concentration of an FGF activator; and
an effective dose of an activator of BMP signaling pathway to induce hepatoblasts;
(d) contacting the cells of step (c) with an effective dose of Hepatocyte Growth Factor (HGF), an effective concentration of Oncostatin-M (OncoM), and an effective concentration of dexamethasone to induce hepatocytes;
(e) contacting the cells to simultaneously induce endothelial cell vasculogenesis and angiogenesis with hepatocyte induction with an effective dose of a VEGF agonist; an effective dose of an activator of the FGF pathway, an effective dose of an inhibitor of the SMAD pathway;
an effective dose of an angiopoietin activating agent;
(f) contacting the cells with an effective dose of a PDGF pathway activating agent; and a TGF-β1 activating agent to simultaneously induce smooth muscle cell differentiation with hepatocyte and endothelial cell differentiation.

15. The method of claim 14, wherein in step (a) the activator of TGF-β pathway is Activin-A (ActA) at an effective dose of from about 10 ng/ml to about 250 ng/ml; the activator of BMP signaling is BMP-4 (BMP4) at an effective dose of from about 1 ng/ml to about 25 ng/ml; the WNT pathway activator is CHIR99021 at an effective dose of from about 1-20 μM; the activator of the FGF pathway is FGF-2 at an effective dose of from about 1 ng/ml to about 20 ng/ml; the PI3K pathway inhibitor is LY294002 at an effective dose of from about 1 μM to about 50 μM.

16. The method of claim 14 or 15, wherein in step (b) the FGF activator is FGF10 at an effective dose of from about 10 ng/ml to about 250 ng/ml.

17. The method of any of claims 14-16, wherein in step (c) the activator of BMP signaling is BMP-4 (BMP4) at an effective dose of from about 1 ng/ml to about 25 ng/ml; and the FGF activator is FGF10 at an effective dose of from about 10 ng/ml to about 250 ng/ml.

18. The method of any of claims 14-17, wherein in step (d) the effective concentration of HGF is from about 10 ng/ml to about 250 ng/ml; the effective concentration of OncoM is from about 10 ng/ml to about 250 ng/m; the effective concentration of DEX is from about 1 μM to about 50 μM.

19. The method of any of claims 14-18, wherein in step (e) the VEGF agonist is VEGF-165 at an effective dose of from about 5 ng/ml to about 100 ng/ml; the activator of the FGF pathway is FGF-2 at an effective dose of from about 1 ng/ml to about 20 ng/ml; the inhibitor of the SMAD pathway is SB431542 at an effective dose of from about 1 μM to about 50 μM; and the angiopoietin activating agent is Angiopoietin-2 (ANG2), or a combination of Angiopoietin-1 and -2 at an effective dose of from about 5 to about 100 ng/ml.

20. The method of any of claims 14-19, wherein in step (f) the PDGF pathway activating agent is PDGF-BB at an effective dose of from about 1 ng/ml to about 25 ng/ml; and the TGF-β1 activating agent is TGF-β1 at an effective dose of from about 0.1 ng/ml to about 5 ng/ml.

21. The method of any of claim 1-5, wherein the vascularized organoid is a neural vascularized organoid, the method comprising simultaneous in vitro co-differentiation of neural cells, and a branching network of endothelial cells and smooth muscle cells.

22. The method of claim 21, comprising the steps of:
(a) contacting the micropatterned PSC with an effective dose of a WNT pathway activating agent; an effective dose of a SMAD pathway inhibitor; an effective dose of dorsomorphin (DM). for a period of from about 24 to about 72 hours;
(b) contacting the cells of step (a) with an effective dose of a WNT pathway activating agent; an effective dose of a SMAD pathway inhibitor; an effective concentration of LIF Interleukin 6 Family Cytokine and an effective concentration of a basic FGF activating agent;
(c) to simultaneously induce endothelial cell vasculogenesis and angiogenesis along with the neural differentiation, contacting the cells with an effective dose of a VEGF agonist; and an effective dose of an angiopoietin agent;
(d) contacting the cells of steps (b) and (c) with an effective concentration a SMAD pathway inhibitor; an effective concentration of a basic FGF activating agent; an effective concentration of a brain derived neurotrophic factor (BDNF), an effective concentration of glial cell line-derived neurotrophic factor.

23. The method of any of claims 21-22, wherein in step (a) the inhibitor of the SMAD pathway is SB431542 at an effective dose of from about 1 μM to about 50 μM; the WNT pathway activator is CHIR99021 (CHIR) at an effective dose of from about 1-20 μM CHIR; and the effective dose of dorsomorphin is from about 1-20 μM.

24. The method of any of claims 21-23, wherein in step (b) the inhibitor of the SMAD pathway is SB431542 at an effective dose from about 1 μM to about 50 μM; the WNT pathway activator is CHIR99021 (CHIR) at an effective dose of from about 1-20 μM; the effective dose of LIF is from about 1 ng/ml to about 20 ng/m; and the bFGF activating factor is bFGF protein, where the effective concentration is from about 1 ng/ml to about 20 ng/ml.

25. The method of any of claims 21-24, wherein in step (d) the inhibitor of the SMAD pathway is SB431542 at an effective dose of from about 1 μM to about 50 μM; the bFGF activating factor is bFGF protein at an effective concentration of from about 1 ng/ml to about 20 ng/ml; the GDNF factor is GDNF protein at an effective concentration of from about 1 ng/ml to about 20 ng/ml; the BDNF factor is BDNF protein at an effective concentration of from about 1 ng/ml to about 20 ng/ml.

26. The method of any of claims 21-25, wherein from about day 7 the culture further comprises an effective dose of a PDGF activating agent.

27. A population of vascularized organoids generated by the method of any of claims 1-26.

28. The population of claim 27, for use in therapeutic transplantation.

29. A method for screening of a candidate agent, the method comprising: contacting the candidate agent with one or a panel of vascularized organoids of claim 27; and determining the effect of the agent on morphologic, genetic, or functional parameters.

30. The method of claim 29, wherein the candidate agent is a drug candidate.

31. The method of claim 29, wherein the candidate agent is a genetic agent.
